SEQUENCE LISTING (62 sequences)

SEQ ID NO: 1 - protein, 1367 aa, Streptococcus
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 4203 bp, Streptococcus
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggagggcagaggaagtcttctaacatgcggtgacgtggaggagaatcccggccct

SEQ ID NO: 3 - DNA, 20 bp, Artificial Sequence
  synthetic construct
gattgtcaactacttgcccc

SEQ ID NO: 4 - DNA, 20 bp, Artificial Sequence
  synthetic construct
ggggcaagtagttgacaatc

SEQ ID NO: 5 - RNA, 20 nt, Artificial Sequence
  synthetic construct
ggggcaaguaguugacaauc

SEQ ID NO: 6 - DNA, 20 bp, Artificial Sequence
  synthetic construct
cggcgagaaagagaatacca

SEQ ID NO: 7 - DNA, 20 bp, Artificial Sequence
  synthetic construct
tggtattctctttctcgccg

SEQ ID NO: 8 - RNA, 20 nt, Artificial Sequence
  synthetic construct
ugguauucucuuucucgccg

SEQ ID NO: 9 - DNA, 20 bp, Artificial Sequence
  synthetic construct
gattccgtactttgcagacg

SEQ ID NO: 10 - DNA, 20 bp, Artificial Sequence
  synthetic construct
cgtctgcaaagtacggaatc

SEQ ID NO: 11 - RNA, 20 nt, Artificial Sequence
  synthetic construct
cgucugcaaaguacggaauc

SEQ ID NO: 12 - DNA, 20 bp, Artificial Sequence
  synthetic construct
cctgagtgtgcatcagctgg

SEQ ID NO: 13 - DNA, 20 bp, Artificial Sequence
  synthetic construct
ccagctgatgcacactcagg

SEQ ID NO: 14 - RNA, 20 nt, Artificial Sequence
  synthetic construct
ccagcugaugcacacucagg

SEQ ID NO: 15 - DNA, 76 bp, Artificial Sequence
  synthetic construct
gttttagagctagaaatagcaagttaaaataaggctagtccgttatcaacttgaaaaagtggcaccgagtcggtgc

SEQ ID NO: 16 - RNA, 76 nt, Artificial Sequence
  synthetic construct
gcaccgacucggugccacuuuuucaaguugauaacggacuagccuuauuuuaacuugcuauuucuagcucuaaaac

SEQ ID NO: 17 - protein, 7 aa, Artificial Sequence
  synthetic construct
PKKKRKV

SEQ ID NO: 18 - DNA, 21 bp, Artificial Sequence
  synthetic construct
ccaaagaagaagcggaaggtc

SEQ ID NO: 19 - protein, 16 aa, Artificial Sequence
  synthetic construct
KRPAATKKAGQAKKKK

SEQ ID NO: 20 - DNA, 48 bp, Artificial Sequence
  synthetic construct
aaaaggccggcggccacgaaaaaggccggccaggcaaaaaagaaaaag

SEQ ID NO: 21 - DNA, 14 bp, Artificial Sequence
  synthetic construct
uuuuuuutuuuuuu

SEQ ID NO: 22 - DNA, 10 bp, Artificial Sequence
  synthetic construct
tttttttttt

SEQ ID NO: 23 - RNA, 10 nt, Artificial Sequence
  synthetic construct
uuuuuuuuuu

SEQ ID NO: 24 - DNA, 768 bp, Artificial Sequence
  synthetic construct
ccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactag

SEQ ID NO: 25 - DNA, 427 bp, Artificial Sequence
  synthetic construct
gaattcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcg

SEQ ID NO: 26 - DNA, 394 bp, Artificial Sequence
  synthetic construct
tcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctg

SEQ ID NO: 27 - DNA, 23 bp, Artificial Sequence
  synthetic construct
gattgtcaactacttgccccagg

SEQ ID NO: 28 - DNA, 23 bp, Artificial Sequence
  synthetic construct
cggcgagaaagagaataccatgg

SEQ ID NO: 29 - DNA, 23 bp, Artificial Sequence
  synthetic construct
gattccgtactttgcagacgagg

SEQ ID NO: 30 - DNA, 23 bp, Artificial Sequence
  synthetic construct
cctgagtgtgcatcagctggtgg

SEQ ID NO: 31 - DNA, 60 bp, Artificial Sequence
  synthetic construct
ggttctcgacggtcacggcgggcatgtcgactcgagaaatttctctggccgttattcgtt

SEQ ID NO: 32 - DNA, 63 bp, Artificial Sequence
  synthetic construct
ccgtcgtggtccttatagtccattttgtttaaacagagttctcttcttgtattcaataattac

SEQ ID NO: 33 - DNA, 60 bp, Artificial Sequence
  synthetic construct
tgtatgctatacgaagttatgctagcggatccgaattcttttttgctcacctgtgattgc

SEQ ID NO: 34 - DNA, 60 bp, Artificial Sequence
  synthetic construct
atttaaatattgcccaattcccaattcccggtaccaaaaaaaaaagcaccgactcggtgc

SEQ ID NO: 35 - DNA, 60 bp, Artificial Sequence
  synthetic construct
aagtggcaccgagtcggtgcttttttttttgcctacctggagcctgagagttgttcaata

SEQ ID NO: 36 - DNA, 60 bp, Artificial Sequence
  synthetic construct
aataacggccagagaaatttctcgactcgagacaaaagctggagctcctgcaggttgttg

SEQ ID NO: 37 - DNA, 60 bp, Artificial Sequence
  synthetic construct
caggagctccagcttttgtctcgagtcgagaaatttctctggccgttattcgttattctc

SEQ ID NO: 38 - DNA, 60 bp, Artificial Sequence
  synthetic construct
tgacctacatcgtcgacactagtggatcaggcctccggccgttaactcgaatcgctatcc

SEQ ID NO: 39 - DNA, 30 bp, Artificial Sequence
  synthetic construct
cacgttatgcgttctcgccacaggatacag

SEQ ID NO: 40 - DNA, 29 bp, Artificial Sequence
  synthetic construct
gcgagtccatttccgatgtgaagtccacg

SEQ ID NO: 41 - DNA, 29 bp, Artificial Sequence
  synthetic construct
ggatgagaatgaacgctggacgaccagac

SEQ ID NO: 42 - DNA, 27 bp, Artificial Sequence
  synthetic construct
ctgtgatctggatctggagcgagtgcg

SEQ ID NO: 43 - DNA, 26 bp, Artificial Sequence
  synthetic construct
ggcttccagctgacccactcgctggg

SEQ ID NO: 44 - DNA, 27 bp, Artificial Sequence
  synthetic construct
ccagatggttcaggtcaccgtaggtgg

SEQ ID NO: 45 - DNA, 20 bp, Artificial Sequence
  synthetic construct
ccaagatcgtgaagaagcgc

SEQ ID NO: 46 - DNA, 20 bp, Artificial Sequence
  synthetic construct
cgcttgttcgatccgtaacc

SEQ ID NO: 47 - DNA, 20 bp, Artificial Sequence
  synthetic construct
cctgaacaaggcctccgatc

SEQ ID NO: 48 - DNA, 20 bp, Artificial Sequence
  synthetic construct
catcgctgccaaactgcttg

SEQ ID NO: 49 - DNA, 19 bp, Streptococcus
ccatatcgtgcctcagagc

SEQ ID NO: 50 - DNA, 21 bp, Streptococcus
gtaatcagcttggcgttcagc

SEQ ID NO: 51 - DNA, 8674 bp, Artificial Sequence
  synthetic construct
ccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgaattcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggattccgtactttgcagacggttttagagctagaaatagcaagttaaaataaggctagtccgttatcaacttgaaaaagtggcaccgagtcggtgcttttttttttggtaccgggaattgggaattgggcaatatttaaatggcggcgcgccgaattcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcgcctgagtgtgcatcagctgggttttagagctagaaatagcaagttaaaataaggctagtccgttatcaacttgaaaaagtggcaccgagtcggtgcttttttttttgcctacctggagcctgagagttgttcaataaaataaaaatgtttcgtttttttgctttcgccagtatttattatttttcatcaatatgtattcaatttggtatgtatttagtaattgtaatatatagacaatggttttccgttgacgtacatacatctgacgtgtgtttatttagacataatagttatgttttcacatctttttaatgttcgcttaatgcgtatgcattctagacaattgtgctcggcaacagtatatttgtggtgtgccaaccaacaacctgcaggagctccagcttttgtctcgagtcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgtttaaacaaaatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccagcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaactagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccggaggcctgatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgac

SEQ ID NO: 52 - DNA, 8674 bp, Artificial Sequence
  synthetic construct
ccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgaattcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggattgtcaactacttgccccgttttagagctagaaatagcaagttaaaataaggctagtccgttatcaacttgaaaaagtggcaccgagtcggtgcttttttttttggtaccgggaattgggaattgggcaatatttaaatggcggcgcgccgaattcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcgcctgagtgtgcatcagctgggttttagagctagaaatagcaagttaaaataaggctagtccgttatcaacttgaaaaagtggcaccgagtcggtgcttttttttttgcctacctggagcctgagagttgttcaataaaataaaaatgtttcgtttttttgctttcgccagtatttattatttttcatcaatatgtattcaatttggtatgtatttagtaattgtaatatatagacaatggttttccgttgacgtacatacatctgacgtgtgtttatttagacataatagttatgttttcacatctttttaatgttcgcttaatgcgtatgcattctagacaattgtgctcggcaacagtatatttgtggtgtgccaaccaacaacctgcaggagctccagcttttgtctcgagtcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgtttaaacaaaatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccagcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaactagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccggaggcctgatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgac

SEQ ID NO: 53 - DNA, 17 bp, Artificial Sequence
  synthetic construct
gtaaaacgacggccagt

SEQ ID NO: 54 - DNA, 19 bp, Artificial Sequence
  synthetic construct
taatacgactcactatagg

SEQ ID NO: 55 - DNA, 478 bp, Artificial Sequence
  synthetic construct
tagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggca

SEQ ID NO: 56 - DNA, 2890 bp, Drosophila melanogaster
atgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaggccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggcactaaccgatccgccgcttctgatctgcgatgagcccacctccggactggactcatttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagcattacattacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgctattagcaaagtagcccgggatatggagcagttgttggccaccaaaaatttggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagttagaattgcctgattccacacccttcttagtttttttcaatgagatgtatagtttatagttttgcagaaaataaataaatttcatttaactcgcgaacatgttgaagatatgaatattaatgagatgcgagtaacattttaatttgcagatggttgccatcttgattggcctcatctttttgggccaacaactcacgcaagtgggcgtgatgaatatcaacggagccatcttcctcttcctgaccaacatgacctttcaaaacgtctttgccacgataaatgtaagtcttgtttagaatacatttgcatattaataatttactaactttctaatgaatcgattcgatttaggtgttcacctcagagctgccagtttttatgagggaggcccgaagtcgactttatcgctgtgacacatactttctgggcaaaacgattgccgaattaccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtag

SEQ ID NO: 57 - protein, 687 aa, Drosophila melanogaster
MGQEDQELLIRGGSKHPSAEHLNNGDSGAASQSCINQGFGQAKNYGTLRPPSPPEDSGSGSGQLAENLTYAWHNMDIFGAVNQPGSGWRQLVNRTRGLFCNERHIPAPRKHLLKNVCGVAYPGELLAVMGSSGAGKTTLLNALAFRSPQGIQVSPSGMRLLNGQPVDAKEMQARCAYVQQDDLFIGSLTAREHLIFQAMVRMPRHLTYRQRVARVDQVIQELSLSKCQHTIIGVPGRVKGLSGGERKRLAFASEALTDPPLLICDEPTSGLDSFTAHSVVQVLKKLSQKGKTVILTIHQPSSELFELFDKILLMAEGRVAFLGTPSEAVDFFSYVGAQCPTNYNPADFYVQVLAVVPGREIESRDRIAKICDNFAISKVARDMEQLLATKNLEKPLEQPENGYTYKATWFMQFRAVLWRSWLSVLKEPLLVKVRLIQTTMVAILIGLIFLGQQLTQVGVMNINGAIFLFLTNMTFQNVFATINVFTSELPVFMREARSRLYRCDTYFLGKTIAELPLFLTVPLVFTAIAYPMIGLRAGVLHFFNCLALVTLVANVSTSFGYLISCASSSTSMALSVGPPVIIPFLLFGGFFLNSGSVPVYLKWLSYLSWFRYANEGLLINQWADVEPGEISCTSSNTTCPSSGKVILETLNFSAADLPLDYVGLAILIVSFRVLAYLALRLRARRKE

SEQ ID NO: 58 - DNA, 4101 bp, Artificial Sequence
  synthetic construct
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 59 - DNA, 54 bp, Artificial Sequence
  synthetic construct
gagggcagaggaagtcttctaacatgcggtgacgtggaggagaatcccggccct

SEQ ID NO: 60 - DNA, 720 bp, Artificial Sequence
  synthetic construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccagcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 61 - DNA, 677 bp, Artificial Sequence
  synthetic construct
ctagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccgg

SEQ ID NO: 62 - DNA, 285 bp, Artificial Sequence
  synthetic construct
gtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgac

(19) United States
(12) Patent Application Publication
(10) Pub. No.: US 2024/0023528 A1
(43) Pub. Date: Jan. 25, 2024

(54) ONE-LOCUS INDUCIBLE PRECISION GUIDED STERILE INSECT TECHNIQUE OR TEMPERATURE-INDUCIBLE PRECISION GUIDED STERILE INSECT TECHNIQUE

(71) Applicant: The Regents of the University of California, Oakland, CA (US)

(72) Inventors: Omar S. Akbari, La Jolla, CA (US); Nikolay Kandul, La Jolla, CA (US)

(21) Appl. No.: 17/926,970

(22) PCT Filed: May 25, 2021
(86) PCT No.: PCT/US2021/034107

§ 371 Date: Nov. 21, 2022

Related U.S. Application Data
(60) Provisional application No. 63/030,222, filed on May 26, 2020.

(51) Int. Cl. A01K 67/033; C12N 15/63; C12N 15/10; C12N 15/113; C12N 9/22

(52) U.S. Cl. CPC A01K 67/0339; C12N 15/635; C12N 15/102; C12N 15/113; C12N 9/22; C07K 2319/09; C12N 2310/20; A01K 2227/706

(57) ABSTRACT

Provided is a gene editing system comprising (i) a polynucleotide encoding an endonuclease under the control of an inducible expressing regulatory sequence; (ii) a guide polynucleotide targeting a female-essential genomic sequence that is required for female-specific viability, and (iii) a guide polynucleotide targeting a male sterility genomic sequence that is required for male-specific fertility. Additionally provided are insect eggs, an insects, and an insect populations, each of which is genetically modified by the gene editing system. Further provided are methods and compositions relating to producing such systems, insect eggs, insects, insect populations and uses thereof in reducing a wild-type insect population.

### CROSS-REFERENCE TO RELATED APPLICATIONS

This application is a U.S. 371 National Stage Application of PCT International Application No. PCT/US2021/034107, filed May 25, 2021, which claims priority under 35 U.S.C. § 119(e) of U.S. Provisional Application No. 63/030,222, filed May 26, 2020, the contents of which are incorporated by reference in its entireties into the present application.

### STATEMENT OF GOVERNMENT SUPPORT

This invention was made with government support under AI151004 awarded by the National Institutes of Health (NIH) and HR0011-17-2-0047 awarded by the Defense Advanced Research Projects Agency (DARPA). The government has certain rights in the invention.

### SEQUENCE LISTING

The instant application contains a Sequence Listing which has been submitted electronically in ASCII format and is hereby incorporated by reference in its entirety. Said ASCII copy, created on Aug. 10, 2023, is named 114198-9811_SL.txt and is 71,350 bytes in size.

### BACKGROUND

Drosophila suzukii (Spotted Wing Drosophila, SWD) is a major invasive pest of ripening small fruit including raspberries, blueberries, strawberries, and cherries. Achieving effective control of D. suzukii has been difficult in a number of crop systems including cherries, and control measures have largely relied on prophylactic application of expensive broad spectrum insecticides. This is problematic, as the repeated use of broad-spectrum insecticides has led to disruption of integrated pest management systems developed for crops such as cherries and berries, and has had a serious impact on beneficial arthropods, resulting in an increased use of miticides. Additionally, the broad use of insecticides makes it inevitable that resistance will become a major problem in the foreseeable future, increases the risk of residues on fruits, and arouses public concern. Similar concerns have been raised with respect to other agricultural pests and human disease vectors.

What remains needed is an efficient, easy-to-practice, cost-effective, fast and safe way to manage infestation and spread of D. suzukii or other insects. This disclosure satisfies these needs and provides related advantages as well.

### SUMMARY OF THE DISCLOSURE

Applicant has developed a one-locus precision guided sterile insect technique (pgSIT) that relies on the inducible expression of Cas9 in a single pure-bred pgSIT line. Such pgSIT method does not require breeding two (Cas9 and gRNA) strains and sex-sorting of parent insects for the genetic cross to generate large numbers of F₁ eggs in a factory. To achieve the positive activation of Cas9 expression, Applicant exploited the promoter of heat-shock protein 70Bb (Hsp70Bb), engineered two separate one-locus pgSIT transgenic lines and demonstrated their performance in Drosophila melanogaster. Though both lines have been pure-bred in the laboratory for more than 10 generations at +18° C., heat-shocking their eggs for 1 hour at +37° C. followed by development at +26° C. consistently resulted in 100% female lethality and male sterility. Since, this system does not require application of any drugs and/or antibiotics, and after a brief heat-shock, insects are maintained under a normal temperature, the pgSIT induction does not affect the fitness of the emerging sterile males. By accomplishing three tasks simultaneously—sex-sorting, female removal, and male sterilization—the one-locus pgSIT technology truly automates the production of large numbers of sterile insect males.

In one aspect, provided herein is a gene editing system that comprises, or consists essentially of, or yet further consists of: (a) a polynucleotide encoding an endonuclease (such as Cas9); (b) an inducible regulatory sequence directing the endonuclease expression in a cell, optionally wherein the cell is an insect germline cell; (c) a guide polynucleotide targeting a female-essential genomic sequence that is required for female-specific viability, or a complementary sequence of the guide polynucleotide, or a polynucleotide expressing the guide polynucleotide; (d) an optional regulatory sequence directing expression of the guide polynucleotide of (c) in a cell; (e) a guide polynucleotide targeting a male sterility genomic sequence that is required for male-specific fertility, or a complementary sequence of the guide polynucleotide, or a polynucleotide expressing the guide polynucleotide; and (f) an optional regulatory sequence directing expression of the guide polynucleotide of (e) in a cell.

In one embodiment, the regulatory sequence of (b) is temperature-sensitive. In a further embodiment, the regulatory sequence of (b) comprises, or consists essentially of, or yet further consists of: a heat-shock protein 70B (Hsp70Bb) promoter.

In another aspect, provided is one or more of: a genetically modified insect egg or a progeny thereof, a genetically modified insect or a progeny thereof, or an insect population comprising, or consisting essentially of, or yet further consisting of at least one genetically modified insect or a progeny thereof. Such genetically modification is performed by the gene editing system as disclosed herein. In one embodiment, the genetically modified insect egg and/or insect comprises the gene editing system as disclosed herein. In a further embodiment, the insect egg or the insect or the insect population or a progeny of each thereof comprises a polynucleotide of pgSIT^{sxl,βTub, Hsp70Bb-Cas9} or a polynucleotide of pg^{SITtraB,βTub, Hsp70Bb-Cas9} as disclosed optionally engineered to one or more of the chromosome(s) or chromosome site(s) of the insect egg or the insect. Additionally or alternatively, expression of an endonuclease in the insect egg or the insect or the insect population or a progeny of each thereof is activated by a heat shock (such as at about 37° C.) and then being kept at a restrictive temperature of the regulatory sequence of (b) (such as about 26° C.). In one embodiment, the insect egg or the insect after activation is a sterile male.

In yet another aspect, provided is a progeny of the genetically modified insect egg, the genetically modified insect, or an insect population comprising, or consisting essentially of, or yet further consisting of at least one genetically modified insect. In one embodiment, the progeny comprises, or consists essentially of, or yet further consists of up to 100% sterile male.

In a further aspect, provided herein is an isolated or engineered polynucleotide comprising, or consisting essentially of, or yet further consisting of any two, any three, any four, any five, or all of (a) to (f) as disclosed herein as well as an isolated or engineered host cell comprising the isolated or engineered polynucleotide. In one embodiment, the isolated or engineered polynucleotide comprises, or consists essentially of, or yet further consists of a polynucleotide of pgSIT^{sxl,βTub, Hsp70Bb-Cas9} or a polynucleotide of pg^{SITtraB,βTub, Hsp70Bb-Cas9} as disclosed. In one embodiment, the host cell is an insect cell. Additionally or alternatively, the host cell is selected from an egg, a sperm, a zygote, or a germline cell.

In one aspect, provided herein is a method of reducing a wild-type insect population comprising, or consisting essentially of, or yet further consisting of introducing an insect egg or an insect or an insect population or a progeny of each thereof as disclosed herein and/or the progeny as disclosed herein, to the wild-type insect population.

In another aspect, provided is a method of producing (1) a genetically modified insect egg, (2) a genetically modified insect, (3) a population comprising the genetically modified insect egg or the genetically modified insect, (4) a population comprising substantially male insect egg or male insect or both, (5) or a progeny of each thereof. The method comprises, or alternatively consists essentially of, or yet further consists of introducing the gene editing system as disclosed herein, or the polynucleotide as disclosed herein, or the vector as disclosed herein into an insect egg, or an insect, or a population of each thereof, or a progeny of each thereof, optionally a wildtype (wt) insect egg, or a wt insect, or a population of each thereof or a progeny of each thereof. In some embodiments, the method further comprises keeping the insect egg, the insect, the population or the progeny comprising the system or the polynucleotide or the vector under a restrictive temperature. In further embodiments, the method further comprises heat shocking the insect egg, the insect, the population, or the progeny comprising the system or the polynucleotide or the vector.

In yet another aspect, provided is a composition comprising, or consisting essentially of, or yet further consisting of a carrier and one or more of: a system as disclosed herein, a polynucleotide as disclosed herein, a vector as disclosed herein, a host cell as disclosed herein, an insect as disclosed herein, an insect egg as disclosed herein, an insect population as disclosed herein, or an insect progeny as disclosed herein.

In a further aspect, provided is a kit comprising, or consisting essentially of, or yet further consisting of an instruction of use in a method as disclosed herein and one or more of: a system as disclosed herein, a polynucleotide as disclosed herein, a vector as disclosed herein, a host cell as disclosed herein, an insect as disclosed herein, an insect egg as disclosed herein, an insect population as disclosed herein, or an insect progeny as disclosed herein.

In some aspects of the disclosed embodiments, the insect is selected from Drosophila melanogaster, Aedes aegypti, Aedes albopictus, Ceratitis capitate, or Drosophila suzukii.

### BRIEF DESCRIPTION OF THE FIGURES

FIG. 1 illustrates a life cycle of insects genetically modified with an exemplified one-locus inducible CRISPR-mediated precision guided Sterile Insect Technique (pgSIT) system.

FIGS. 2A-2C provide schematic of genetic constructs used in the examples. As shown in FIG. 2A, the Drosophila heat-shock protein 70B (Hsp70Bb) promoter directs the temperature-inducible expression of Cas9. The coding sequence of the Streptococcus pyogenes-derived Cas9 (Cas9) was flanked by two nuclear localization signals (NLS) at both ends, to promote nuclear localization, and a self-cleaving T2A peptide with GFP coding sequence at the C-terminal end, serving as a visual indicator of Cas9 expression. The Opie2-dsRed-SV40 marker transgene was included in the Hsp70Bb-Casal9 constructs. FIG. 2B shows double guide RNA (dgRNA) genetic constructs. The constitutive expression of two gRNAs targeting βTubulin 85D (βTub) and either sex lethal (sxl) or transformer (tra) is achieved by the Drosophila U6.3 promoter. The dgRNA constructs are tracked by the mini-white marker gene. The gRNA sequences are indicated in the Table 1. FIG. 2C shows Temperature-Inducible precision guided Sterile Insect Technique (TI-pgSIT) genetic cassettes. The Hsp-70Bb-Cas9-T2A-GFP-p10 fragment was added to the two dgRNA constructs to build two TI-pgSIT cassettes. The genetic cassettes were site-specifically integrated at the P{CaryP}attP2 site on the 3rd chromosome (BDSC #8622). The genetic constructs and Drosophila transgenic lines generated in the study were deposited at Addgene.org and the Bloomington Drosophila Stock Center.

FIGS. 3A-3E provide assessment of temperature inducible pgSIT systems. To establish a visual indicator of Cas9 expression, the GFP coding sequence was attached to the C-terminal end of the Streptococcus pyogenes-derived Cas9 (Cas9) coding sequence via a self-cleaving T2A peptide. As shown in FIGS. 3A-3B, a two-hour heat shock at 37° C. activates the expression of Hsp70Bb-Cas9 at the P{CaryP}attP2 site, as indicated by the GFP expression. FIG. 3B shows that raising embryos harboring the Hsp70Bb-Cas9 to adult flies at 26° C. does not activate visible GFP fluorescence in living flies. The baseline and activated expression of Hsp70Bb-Cas9 was tested in combination with three different dgRNAs: (FIG. 3C) dgRNA^{sxl,βTub}, (FIG. 3D) dgRNA^{traA,βTub} and (FIG. 3E) dgRNA^{traB,βTub} to assess the feasibility of the Temperature-Inducible precision guided Sterile Insect Technique (TI-pgSIT) design. The staged trans-heterozygous F₁ embryos generated by reciprocal genetic crosses between homozygous dgRNAs and Hsp70Bb-Cas9 lines were raised at 18° C. or 26° C. with additional heat shocks at 37° C. The sex and fertility of emerged adult flies was scored and plotted as bar graphs. Since the knockouts of sxl and tra transform the normal-looking females into intersexes, the emerging F₁ flies were scored as females (♀, the left bar of each set), males (♂, the middle bar of each set), or intersexes ( [symbol] , the right bar of each set). The frequency of each sex that emerged under a specific temperature condition was compared to that of the same sex that emerged at 18° C. without a heat shock. Additionally, the male frequency was compared to the female and intersex frequency for each condition. Bar plots show the mean±standard deviation (SD) over at least three biological replicates. Statistical significance in sex frequency was estimated using a two-sided Student's t test with equal variance. (^{ns}p≥0.05, *p<0.05, **p<0.01, and ***p<0.001).

FIGS. 4A-4C show that at 18° C., Cas9 protein carryover induced by maternal Hsp70Bb-Cas9 does not affect F₁ sex frequencies. To explore whether a leaky Cas9 expression under 18° C. causes the maternal protein carryover affecting the progeny sex frequencies, homozygous Hsp70Bb-Cas9 line was genetically crossed to each of three homozygous dgRNA lines in both directions and sex frequencies of F₁ trans-heterozygotes harboring Cas9 inherited from mothers (maternal Cas9, Hsp70Bb-Cas9 ♀ x dgRNA ♂) or fathers (paternal Cas9, Hsp70Bb-Cas9 ♂ x dgRNA ♀) were compared. Significant differences was not identified in sex frequencies between progenies harboring maternal vs paternal Cas9 reared under 18° C. (Table 2). These findings suggest that the basal expression of Hsp70Bb-Cas9 does not cause the Cas9 maternal carryover affecting the progeny sex frequencies. Bar plots show the mean±SD over five biological replicates. Statistical significance in sex frequency was estimated using a two-sided Student's t test with equal variance. (^{ns}p≥0.05).

For each set of bars, the left one indicates female, the middle one indicates male, while the right one indicates intersex.

FIGS. 5A-5E show that elevating the temperature of one-locus TI-pgSIT lines produces desired phenotypes. Assessment of phenotypes upon temperature treatments comparing two single locus TI-pgSIT cassettes, (FIG. 5A) TI-dgRNA^{sxl,βTub,Hsp-Cas9} and (FIG. 5B, heterozygous; FIG. 5C, homozygous) dgRNA^{traB,βTub,Hsp-Cas9}. At 18° C., transgenic flies harboring one or two copies of the TI-pgSIT cassette produced both females and males at a nearly equal sex ratios and can be pure-bred for many generations. The full activation of the TI-pgSIT cassette was achieved by raising the flies at 26° C. with an additional heat-shock at 37° C. during the first days of development. This activating temperature condition induced 100% penetrance of the pgSIT phenotypes, female-specific lethality and male-specific sterility, and as a result, only sterile males emerge. The sex and fertility of emerged adult flies was scored and plotted as bar graphs. The emerging flies were scored as females (♀, the left bar of each set), males (♂), or intersexes ( [symbol] , the right bar of each set). The middle bar of each set represents fertile male, sterile male, or sterile male or intersex (labeled as Sterile M or I) as noted in the figures. The frequency of each sex that emerged under 18° C. treatment was compared to that of the same sex. Additionally, the male frequency was compared to the female and intersex frequency under each condition. Bar plots show the mean±SD over at least three biological replicates. Statistical significance in sex frequency was estimated using a two-sided Student's t test with equal variance. Pearson's chi-squared tests for contingency tables were used to assess the difference in male sterility. (^{ns}p≥0.05, *p<0.05, **p<0.01, and ***p<0.001). As shown in FIGS. 5D-5E, notably, after close examination of heat-shock-induced dgRNA^{traB,βTub,Hsp-Cas9} males, a fraction of flies referred to as males were indeed intersexes. These intersexes have very similar external morphology, including abdomen pigmentation (FIG. 5E₁₋₂), genitals (FIG. 5E₃), and sex combs (FIG. 5E₃), to that of males (FIG. 5D₁₋₄) prohibiting their correct identification. Some older intersexes can be identified when, instead of testes (FIG. 5D₅), they develop ovaries (FIG. 5E₅), which result in abdomen extension (FIG. 5E₂ vs FIG. 5D₂).

FIGS. 6A-6B provide basal Hsp70Bb-Cas9 expression in somatic tissues of TI-pgSIT^{sxl,βTub,Hsp-Cas9} flies. The Drosophila heat-shock protein 70B (Hsp70Bb) promoter is known to drive a baseline expression at 25° C. (Steller & Pirrotta; Bishop & Corces; and Bang & Posakony). To assess whether Hsp70Bb-Cas9 is expressed at 18° C., target sites in sxl (FIG. 6A) and βTub (FIG. 6B) were sequenced using the DNA extracted from multiple females (Σ♀) and males (Σ♂) of TI-pgSIT^{sxl,βTub,Hsp-Cas9}/+ flies reared at 20° C. The presence of induced indel alleles at the cut site (indicated by lines) among wt alleles for both sxl and βTub loci caused ambiguity (multiple peaks) of sequence reads. Directions of sequence reads are indicated with arrows (forward primer—arrows pointing to the right; reverse primer—arrows pointing to the left).

FIGS. 7A-7C show stability and performance of the TI-pgSIT system twelve months after its development. FIG. 7A provides a re-assessment of TI-pgSIT^{sxl,βTub,Hsp-Cas9} and TI-pgSIT^{traB,βTub,Hsp-Cas9} one-locus TI-pgSIT lines 12 months later. The sex and fertility of emerged adult flies was scored and plotted as bar graphs. The emerging flies were scored as females (♀, the left bar of each set), males (♂), or intersexes ( [symbol] , the right bar of each set), and numbers of scored flies are indicated for each bar. The middle bar of each set represents sterile male (marked as “Sterile M”), or sterile male or intersex (marked as “Sterile M/I”), or fertile male (not marked with “Sterile M” or “Sterile M/I”). Eggs were collected at 18° C. and 26° C., and emerging larvae were heat-shocked at 37° C. for 2 hours and then reared at 26° C. The frequency of each sex and its fertility was compared to those of the corresponding sexes reared at 18° C. Additionally, the male frequency was compared to the female and intersex frequency under each condition. Bar plots show the mean±SD over at least three biological replicates. Statistical significance in sex frequency was estimated using a two-sided Student's t test with equal variance. (^{ns}p≥0.05, and ***p<0.001). (FIG. 7B) In TI-pgSIT^{sxl,βTub,Hsp-Cas9} line, Cas9 transcription increases nearly 1000 times after a two-hour heat-shock at 37° C. Total RNA was extracted from 2nd instar larvae four hours after heat shock, and RT-qPCR quantification of Cas9 expression was done relative to RPL32 and ATPsynCF6. As shown in FIG. 7C, the heat induced TI-pgSIT^{sxl,βTub,Hsp-Cas9} males successfully competed against wt males to secure matings with wt females. The mating success of sterile TI-pgSIT males was evaluated by fecundity decrease (aka. increase of unhatched egg rate). D. melanogaster mated female was resistant to the next mating for around 24 hours (Peng et al., Curr. Biol. 15, 207-213 (2005); and Manning. Animal Behaviour vol. 10 384-385 (1962)). Therefore, ten virgin females were confined with multiple males for 12 hours before removing males and assessing female fecundity. The mean percentage of hatched eggs and ±SD over five biological replicates are indicated on the bar graph. It was previously showed that a single fertile male was able to fertilize the majority of ten virgin females during 12 hours (Kandul et al. 2019). The addition of five or ten TI-pgSIT sterile males to five fertile wt males resulted in a significant decrease in female fecundity, 92±3% vs 32±7 or 27±5%, respectively. Statistical significance was estimated using a two-sided Student's t test with equal variance. (^{ns}p≥0.05, and ***p<0.001).

FIGS. 8A-8C provide schematic of genetic constructs built and tested in the study. As shown in FIG. 8A, the Drosophila Hsp70Bb promoter directs the temperature-inducible expression of Cas9. The heat-shock protein 70B (Hsp70Bb) has been used for the inducible expression of Drosophila transgenes by a heat-shock at 37° C. for nearly 20 years (Thummel & Pirotta. PNAS, 99, 7877-7882 (1992)). The coding sequence of the Streptococcus pyogenes-derived Cas9 (SpCas9) was flanked by two nuclear localization signals (NLS) at both ends and a self-cleaving T2A peptide with GFP coding sequence at the C-end, serving as a visual indicator of Cas9 expression. Opie2-dsRed-SV40 a body-specific marker transgene was included into the Hsp70Bb-Cas9 constructs. FIG. 8B provides double guide RNA (gdRNA) genetic constructs. The constitutive expression of two gRNAs targeting sex lethal (sxl) or transformer (tra) together βTubiline 85D (βTub) is achieved by the Drosophila U6.3 promoter. The dgRNAs constructs are tracked by the mini-white marker gene. FIG. 8C provides one-locus pgSIT genetic cassettes. The Hsp-70Bb-Cas9-T2A-GFP-p10 fragment was added to the two dgRNAs constructs to build two one-locus pgSIT cassettes. The genetic constructs stored at Addgene.org were site-specifically integrated at one of three attP sites in the Drosophila genome and deposited to Bloomington Drosophila Stock Center.

FIGS. 9A-9C provide assessment of inducible split-pgSIT systems. To assess the feasibility of one-locus precision guided Sterile Insect Technique (pgSIT) design, the basic and activated expression of Hsp70Bb-Cas9 was tested in combination with three different dgRNAs: dgRNA sxl,βTub (FIG. 9A), dgRNA tra,βTub (FIG. 9B), and dgRNA traB,βTub (FIG. 9C). The staged trans-heterozygous F₁ embryos generated by the genetic cross between homozygous dgRNAs and Hsp70Bb-Cas9 lines were raised at 20° C. or 26° C. with additional heat-shocks at 37° C. The sex and fertility of emerged adult flies was scored and plotted as bar graphs. Since the knockouts of sxl and tra transform the normal-looking females into intersexes, the emerging F₁ flies were scored as females (♀, left bar of each set), males (♂, middle bar of each set), or intersexes ( [symbol] , right bar of each set). The frequency of each sex emerged under a specific temperature treatment was compared to that of the same sex emerged under 20° C. without any heat-shock. In addition, the male frequency was compared to that of females and intersexes under each treatment. Bar plots show the mean±SD over at least three biological replicates. Statistical significance in sex frequency was estimated using a two-sample Student's t test with equal variance. Pearson's chi-squared tests for contingency tables were used to assess the difference in male sterility. (P≥0.05 ns, P<0.05*, P<0.01**, and P<0.001***)

FIGS. 10A-10C show one-locus inducible pgSIT lines. Two different pure-breeding one-locus precision guided Sterile Insect Technique (pgSIT) transgenic lines were generated using two one-locus pgSIT cassettes, dgRNA sxl,βTub,Hsp70Bb-Cas9 (FIG. 10A) and dgRNA traB,βTub,Hsp70Bb-Cas9 (FIGS. 10B-10C). Under the permissive temperature of 18° C., the transgenic flies harboring one or two copies of one-locus pgSIT cassette produce both females and males at a nearly normal sex ratio and can be pure-bred for many generations. The full activation of the one-locus pgSIT cassette is achieved by raising the transgenic flies under the restrictive temperature of 26° C. with an additional heat-shock at 37° C. during the first days of development. This activating temperature profile induces 100% penetrance of the pgSIT phenotypes, female-specific lethality and male-specific sterility, and only 100% sterile males emerge. The sex and fertility of emerged adult flies was scored and plotted as bar graphs. Since the knockouts of sxl and tra transform the normal-looking females into intersexes, the emerging F₁ flies were scored as females (♀, left bar of each set), males (♂, middle bar of each set), or intersexes ( [symbol] , right bar of each set). The frequency of each sex emerged under the activating temperature treatment was compared to that of the same sex emerged under 18° C. In addition, the male frequency was compared to that of females and intersexes under each treatment. Bar plots show the mean±SD over at least three biological replicates. Statistical significance in sex frequency was estimated using a two-sample Student's t test with equal variance. Pearson's chi-squared tests for contingency tables were used to assess the difference in male sterility. (P≥0.05 ns, P<0.05*, P<0.01**, and P<0.001***).

FIGS. 11A-11C provide an exemplified plasmid map of TI-pgSIT[traB,bTUb,Hsp70Bb-Cas9] (FIG. 11A) and an exemplified sequence of TI-pgSIT[traB,bTUb,Hsp70Bb-Cas9] (FIGS. 11B-11C). FIGS. 11B-11C disclose SEQ ID NO: 51.

FIGS. 12A-12C provide an exemplified plasmid map of TI-pgSIT[sxl,bTUb,Hsp70Bb-Cas9] (FIG. 12A) and an exemplified sequence of TI-pgSIT[sxl,bTUb,Hsp70Bb-Cas9] (FIGS. 12B-12C). FIGS. 12B-12C disclose SEQ ID NO: 52.

FIGS. 13A-13D provides a table listing exemplified sequences of TI-pgSIT components. FIG. 13A discloses SEQ ID NOs: 53, 54, 55, and 56, respectively, in order of appearance. FIG. 13B discloses SEQ ID NOs: 57, 24, 25, 3, 9, 12, 15, 22, and 26, respectively, in order of appearance. FIG. 13C discloses SEQ ID NOS: 18, 58, 20, 59, 60, 61, and 62, respectively, in order of appearance.

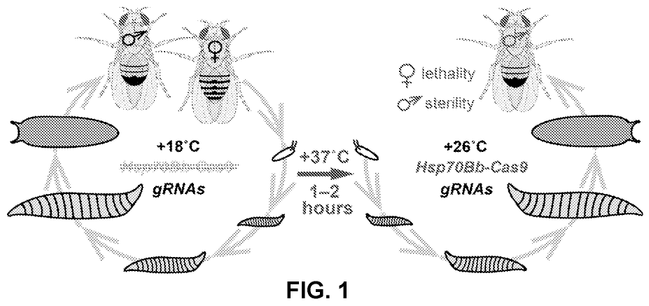

### DETAILED DESCRIPTION

### Definitions

As it would be understood, the section or subsection headings as used herein is for organizational purposes only and are not to be construed as limiting and/or separating the subject matter described.

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, hbbb Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R.I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As will be understood by one skilled in the art, for any and all purposes, all ranges disclosed herein also encompass any and all possible subranges and combinations of subranges thereof. Furthermore, as will be understood by one skilled in the art, a range includes each individual member.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

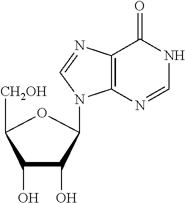

Inosine is read by the translation machinery as guanine (G).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “functional” may be used to modify any molecule, biological, or cellular material to intend that it accomplish a particular, specified effect.

The compositions for the administration of the CRISPR vectors and systems can be conveniently presented in dosage unit form and can be prepared by any of the methods well known in the art.

“Messenger RNA” or “mRNA” is a nucleic acid molecule that is transcribed from DNA and then processed to remove non-coding sections known as introns. The resulting mRNA is exported from the nucleus (or another locus where the DNA is present) and translated into a protein. The term “pre-mRNA” refers to the strand prior to processing to remove non-coding sections.

The terms “hairpin,” “hairpin loop,” “stem loop,” and/or “loop” used alone or in combination with “motif” is used in context of an oligonucleotide to refer to a structure formed in single stranded oligonucleotide when sequences within the single strand which are complementary when read in opposite directions base pair to form a region whose conformation resembles a hairpin or loop.

As used herein, the term “domain” refers to a particular region of a protein or polypeptide and is associated with a particular function. For example, “a domain which associates with an RNA hairpin motif” refers to the domain of a protein that binds one or more RNA hairpin. This binding may optionally be specific to a particular hairpin.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein editing techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence that can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

As used herein, the term “recombinant expression system” refers to a genetic construct or constructs for the expression of certain genetic material formed by recombination.

A “vector” is defined as any molecule that can carry inserted polynucleotides into a host cell. Examples of vectors are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression. In some embodiments, the vector is a non-viral vector, such as a plasmid. In some embodiments, the vector is a viral vector.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, a “contiguous” polynucleotide refers to nucleic acid sequence conjugated with each other directly or indirectly.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro.

Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. Proc. Nat. Acad. Sci. USA 106(15):6099-6104 (2009)). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. Further details as to modern methods of vectors for use in gene transfer may be found in, for example, Kotterman et al. (2015) Viral Vectors for Gene Therapy: Translational and Clinical Outlook Annual Review of Biomedical Engineering 17.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. Ads do not require integration into the host cell genome. Recombinant Ad derived vectors, particularly those that reduce the potential for recombination and generation of wild-type virus, have also been constructed. Such vectors are commercially available from sources such as Takara Bio USA (Mountain View, CA), Vector Biolabs (Philadelphia, PA), and Creative Biogene (Shirley, NY). Wild-type AAV has high infectivity and specificity integrating into the host cell's genome. See, Wold and Toth (2013) Curr. Gene. Ther. 13(6):421-433, Hermonat & Muzyczka (1984) Proc. Natl. Acad. Sci. USA 81:6466-6470, and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, a “gene editing system” refers to refers to genetic engineering in which a pol nucleotide is inserted, deleted, modified or replaced in a cell, optionally of an insect.

As used herein, the term “helper” in reference to a virus or plasmid refers to a virus or plasmid used to provide the additional components necessary for replication and packaging of a viral particle or recombinant viral particle. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise a nucleic acid molecule encoding γ-sarcoglycan (e.g., SEQ ID NO: 1) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, International Published Application No. WO 2001/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

The term “Cas9” refers to a CRISPR associated endonuclease referred to by this name. Non-limiting exemplary Cas9s are provided herein, e.g. the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112. In some embodiments, spCas9 comprises, or consists essentially of, or yet further consists of a sequence disclosed as UniProtKB Q99ZW2, or P66670, or J7M7J1, the sequence of each of which is enclosed herein by reference in its entirety, last accessed on May 24, 2021. Additionally amino acid or nucleotide sequences of an endonuclease are available to one of skill in the art, see for example, U.S. Pat. Nos. 8,945,839, 9,790,490, 10,377,998, 10,946,108, 10,577,630, International Published Application No. WO 2021/016600, and U.S. Pat. No. 10,550,372.

In some embodiments, the Cas9 protein comprises, or consists essentially of, or yet further consists of

(SEQ ID NO: 1)
DKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSF
FHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYN
QLFEENPINASGVDAKAILSARLSKSRRLENLIAQLPGEKKNGLFGNL
IALSLGLTPNFKSNFDLAEDAKLQLSKDTYDDDLDNLLAQIGDQYADL
FLAAKNLSDAILLSDILRVNTEITKAPLSASMIKRYDEHHQDLTLLKA
LVRQQLPEKYKEIFFDQSKNGYAGYIDGGASQEEFYKFIKPILEKMDG
TEELLVKLNREDLLRKQRTFDNGSIPHQIHLGELHAILRRQEDFYPFL
KDNREKIEKILTFRIPYYVGPLARGNSRFAWMTRKSEETITPWNFEEV
VDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTL
FEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLH
EHIANLAGSPAIKKGILQTVKVVDELVKVMGRHKPENIVIEMARENQT
TQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRG
KSDNVPSEEVVKKMKNYWRQLLNAKLITQRKFDNLTKAERGGLSELDK
AGFIKRQLVETRQITKHVAQILDSRMNTKYDENDKLIREVKVITLKSK
LVSDFRKDFQFYKVREINNYHHAHDAYLNAVVGTALIKKYPKLESEFV
YGDYKVYDVRKMIAKSEQEIGKATAKYFFYSNIMNFFKTEITLANGEI
RKRPLIETNGETGEIVWDKGRDFATVRKVLSMPQVNIVKKTEVQTGGF
SKESILPKRNSDKLIARKKDWDPKKYGGFDSPTVAYSVLVVAKVEKGK
SKKLKSVKELLGITIMERSSFEKNPIDFLEAKGYKEVKKDLIIKLPKY
SLFELENGRKRMLASAGELQKGNELALPSKYVNFLYLASHYEKLKGSP
EDNEQKQLFVEQHKHYLDEIIEQISEFSKRVILADANLDKVLSAYNKH
RDKPIREQAENIIHLFTLTNLGAPAAFKYFDTTIDRKRYTSTKEVLDA
TLIHQSITGLYETRIDLSQLGGD.

In further embodiments, the Cas9 is encoded by a polynucleotide comprising, or consisting essentially of, or yet further consisting of

(SEQ ID NO: 2)
gacaagaagtacagcatcggcctggacatcggcaccaactctgtgggc
tgggccgtgatcaccgacgagtacaaggtgcccagcaagaaattcaag
gtgctgggcaacaccgaccggcacagcatcaagaagaacctgatcgga
gccctgctgttcgacagcggcgaaacagccgaggccacccggctgaag
agaaccgccagaagaagatacaccagacggaagaaccggatctgctat
ctgcaagagatcttcagcaacgagatggccaaggtggacgacagcttc
ttccacagactggaagagtccttcctggtggaagaggataagaagcac
gagcggcaccccatcttcggcaacatcgtggacgaggtggcctaccac
gagaagtaccccaccatctaccacctgagaaagaaactggtggacagc
accgacaaggccgacctgcggctgatctatctggccctggcccacatg
atcaagttccggggccacttcctgatcgagggcgacctgaaccccgac
aacagcgacgtggacaagctgttcatccagctggtgcagacctacaac
cagctgttcgaggaaaaccccatcaacgccagcggcgtggacgccaag
gccatcctgtctgccagactgagcaagagcagacggctggaaaatctg
atcgcccagctgcccggcgagaagaagaatggcctgttcggaaacctg
attgccctgagcctgggcctgacccccaacttcaagagcaacttcgac
ctggccgaggatgccaaactgcagctgagcaaggacacctacgacgac
gacctggacaacctgctggcccagatcggcgaccagtacgccgacctg
tttctggccgccaagaacctgtccgacgccatcctgctgagcgacatc
ctgagagtgaacaccgagatcaccaaggcccccctgagcgcctctatg
atcaagagatacgacgagcaccaccaggacctgaccctgctgaaagct
ctcgtgcggcagcagctgcctgagaagtacaaagagattttcttcgac
cagagcaagaacggctacgccggctacattgacggcggagccagccag
gaagagttctacaagttcatcaagcccatcctggaaaagatggacggc
accgaggaactgctcgtgaagctgaacagagaggacctgctgcggaag
cagcggaccttcgacaacggcagcatcccccaccagatccacctggga
gagctgcacgccattctgcggcggcaggaagatttttacccattcctg
aaggacaaccgggaaaagatcgagaagatcctgaccttccgcatcccc
tactacgtgggccctctggccaggggaaacagcagattcgcctggatg
accagaaagagcgaggaaaccatcaccccctggaacttcgaggaagtg
gtggacaagggcgcttccgcccagagcttcatcgagcggatgaccaac
ttcgataagaacctgcccaacgagaaggtgctgcccaagcacagcctg
ctgtacgagtacttcaccgtgtataacgagctgaccaaagtgaaatac
gtgaccgagggaatgagaaagcccgccttcctgagcggcgagcagaaa
aaggccatcgtggacctgctgttcaagaccaaccggaaagtgaccgtg
aagcagctgaaagaggactacttcaagaaaatcgagtgcttcgactcc
gtggaaatctccggcgtggaagatcggttcaacgcctccctgggcaca
taccacgatctgctgaaaattatcaaggacaaggacttcctggacaat
gaggaaaacgaggacattctggaagatatcgtgctgaccctgacactg
tttgaggacagagagatgatcgaggaacggctgaaaacctatgcccac
ctgttcgacgacaaagtgatgaagcagctgaagcggcggagatacacc
ggctggggcaggctgagccggaagctgatcaacggcatccgggacaag
cagtccggcaagacaatcctggatttcctgaagtccgacggcttcgcc
aacagaaacttcatgcagctgatccacgacgacagcctgacctttaaa
gaggacatccagaaagcccaggtgtccggccagggcgatagcctgcac
gagcacattgccaatctggccggcagccccgccattaagaagggcatc
ctgcagacagtgaaggtggtggacgagctcgtgaaagtgatgggccgg
cacaagcccgagaacatcgtgatcgaaatggccagagagaaccagacc
acccagaagggacagaagaacagccgcgagagaatgaagcggatcgaa
gagggcatcaaagagctgggcagccagatcctgaaagaacaccccgtg
gaaaacacccagctgcagaacgagaagctgtacctgtactacctgcag
aatggggggatatgtacgtggaccaggaactggacatcaaccggctgt
ccgactacgatgtggaccatatcgtgcctcagagctttctgaaggacg
actccatcgacaacaaggtgctgaccagaagcgacaagaaccggggca
agagcgacaacgtgccctccgaagaggtcgtgaagaagatgaagaact
actggcggcagctgctgaacgccaagctgattacccagagaaagttcg
acaatctgaccaaggccgagagaggcggcctgagcgaactggataagg
ccggcttcatcaagagacagctggtggaaacccggcagatcacaaagc
acgtggcacagatcctggactcccggatgaacactaagtacgacgaga
atgacaagctgatccgggaagtgaaagtgatcaccctgaagtccaagc
tggtgtccgatttccggaaggatttccagttttacaaagtgcgcgaga
tcaacaactaccaccacgcccacgacgcctacctgaacgccgtcgtgg
gaaccgccctgatcaaaaagtaccctaagctggaaagcgagttcgtgt
acggcgactacaaggtgtacgacgtgcggaagatgatcgccaagagcg
agcaggaaatcggcaaggctaccgccaagtacttcttctacagcaaca
tcatgaactttttcaagaccgagattaccctggccaacggcgagatcc
ggaagcggcctctgatcgagacaaacggcgaaaccggggagatcgtgt
gggataagggccgggattttgccaccgtgcggaaagtgctgagcatgc
cccaagtgaatatcgtgaaaaagaccgaggtgcagacaggcggcttca
gcaaagagtctatcctgcccaagaggaacagcgataagctgatcgcca
gaaagaaggactgggaccctaagaagtacggcggcttcgacagcccca
ccgtggcctattctgtgctggtggtggccaaagtggaaaagggcaagt
ccaagaaactgaagagtgtgaaagagctgctggggatcaccatcatgg
aaagaagcagcttcgagaagaatcccatcgactttctggaagccaagg
gctacaaagaagtgaaaaaggacctgatcatcaagctgcctaagtact
ccctgttcgagctggaaaacggccggaagagaatgctggcctctgccg
gcgaactgcagaagggaaacgaactggccctgccctccaaatatgtga
acttcctgtacctggccagccactatgagaagctgaagggctcccccg
aggataatgagcagaaacagctgtttgtggaacagcacaagcactacc
tggacgagatcatcgagcagatcagcgagttctccaagagagtgatcc
tggccgacgctaatctggacaaagtgctgtccgcctacaacaagcacc
gggataagcccatcagagagcaggccgagaatatcatccacctgttta
ccctgaccaatctgggagcccctgccgccttcaagtactttgacacca
ccatcgaccggaagaggtacaccagcaccaaagaggtgctggacgcca
ccctgatccaccagagcatcaccggcctgtacgagacacggatcgacc
tgtctcagctgggaggcgacaaaaggccggcggccacgaaaaaggccg
gccaggcaaaaaagaaaaaggagggcagaggaagtcttctaacatgcg
gtgacgtggaggagaatcccggccct.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

As used herein, with respect to the CRISPR-based technology, the term “guide polynucleotide” refers to a polynucleotide having a “synthetic sequence” capable of binding the corresponding endonuclease enzyme protein (e.g., Cas9) and a variable target sequence capable of binding the genomic target (e.g., a nucleotide sequence found in an exon of a target gene). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences.

With respect to the endonuclease enzyme protein of the CRISPR-based technology, the term “endonuclease” refers to any suitable endonuclease enzyme protein or a variant thereof that will be specifically directed by the selected guide polynucleotide to enzymatically knock-out the target sequence of the guide polynucleotide.

As used herein, the term “variant thereof,” as used with respect to an endonuclease, refers to the referenced endonuclease in its enzymatically functional form expressed in any suitable host organism or expression system and/or including any modifications to enhance the enzymatic activity of the endonuclease.

In some embodiments of the present disclosure, a suitable endonuclease includes a CRISPR-associated sequence 9 (Cas9) endonuclease or a variant thereof, a CRISPR-associated sequence 13 (Cas13) endonuclease or a variant thereof, CRISPR-associated sequence 6 (Cas6) endonuclease or a variant thereof, a CRISPR from Prevotella and Francisella 1 (Cpf1) endonuclease or a variant thereof, or a CRISPR from Microgenomates and Smithella 1 (Cms1) endonuclease or a variant thereof. In some embodiments of the present disclosure, a suitable endonuclease includes a Streptococcus pyogenes Cas9 (SpCas9), a Staphylococcus aureus Cas9 (SaCas9), a Francisella novicida Cas9 (FnCas9), or a variant thereof. Variants may include a protospacer adjacent motif (PAM) SpCas9 (xCas9), high fidelity SpCas9 (SpCas9-FIF1), a high fidelity SaCas9, or a high fidelity FnCas9. In other embodiments of the present disclosure, the endonuclease comprises, or alternatively consists essentially of, or yet further consists of a Cas fusion nuclease comprising, or alternatively consisting essentially of, or yet further consisting of a Cas9 protein or a variant thereof fused with a Fok1 nuclease or variant thereof. Variants of the Cas9 protein of this fusion nuclease include, but are not limited to a catalytically inactive Cas9 (e.g., dead Cas9).

In some embodiments of the present disclosure, the endonuclease may be a Cas9, Cas13, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM 10152, Rhodococcus erythropolis PR4, Rhodococcus jostii RFIA1, Rhodococcus opacus B4 (uid36573), Acidothermus cellulolyticus 11B, Arthrobacter chlorophenolicus A6, Kribbella flavida (DSM 17836, uid43465), Thermomonospora curvata (DSM 43183), Bifidobacterium dentium Bd1, Bifidobacterium longum DJO10A, Slackia heliotrinireducens (DSM 20476), Persephonella marina EX-H1, Bacteroides fragilis NCTC 9434, Capnocytophaga ochracea (DSM 7271), Flavobacterium psychrophilum JIP02/86, Akkermansia muciniphila (ATCC BAA-835), Roseiflexus castenholzii (DSM 13941), Roseiflexus RS-1, Synechocystis PCC6803, Elusimicrobium minutum Pei191, uncultured Termite group 1 bacterium phylotype Rs-D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 (JGI), Azospirillum B51 0 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269.97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U112, Francisella tularensis holarctica, Francisella tularensis FSC 198, Francisella tularensis, Francisella tularensis WY96-3418, or Treponema denticola (ATCC 35405).

The term “cell” as used herein may refer to either a prokaryotic or eukaryotic cell, optionally obtained from a subject or a commercially available source. In one embodiment, the cell is an insect cell.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, Chinese Hamster Ovary (CHO) cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the terms “engineered” “modified” and like terms refers to the introduction of a heterologous recombinant nucleic acid sequence into the target, such as another nucleic acid sequence, chromosome, cell or insect egg, or insect. As would be understood by one of ordinary skill in the art, techniques for genetic modification of insects are known and described, for example in Cockburn et al., Biotechnology and Genetic Engineering Reviews, 2: 68-99, (1984), the entire contents of which are incorporate herein by reference. In one embodiment, the term “engineered” “integrated” “modified” or the like may refer to the integration of recombinant nucleic acid sequence into the genome of the target insect. The genome of the target insect includes at least one chromosome of the target insect, but may include all relevant chromosome copies. As such, integration into the genome may be heterozygous or homozygous.

As used herein, the term “female-essential genomic sequence” encompasses any genomic sequence or gene specific to the female insect. Examples of a female-essential genomic sequence include a sex-determination gene or a female-specific splice variant thereof, a gene or splice variant of a gene not found in the male, a gene or splice variant of a gene essential for female gonadal development, and/or a gene or splice variant of a gene not essential for male viability. Non-limiting examples of female-essential genomic sequences include the female-specific exons in the sex-determination Drosophila genes Sxl, Tra, and Dsx including homologs, orthologs, and paralogs thereof. As used herein, the term “homolog” refers to the comparable gene of an organism found in another organism conferring the same function. As used herein, the terms “orthologs” and “paralogs” refer to types of homologs. Orthologs are corresponding genes in different lineages and are a result of speciation, and paralogs result from a gene duplication. See, for example, International Published Application No. WO 2019/103982.

As used herein, the term “male sterility genomic sequence” refers to any male-specific genomic sequence required for male fertility in an insect which does not affect the development of the male insect or the viability of the male insect. Non-limiting examples of a male-specific genomic sequence required for male fertility in an insect include the genes [[p]]βTubulin 85D ([[P]]βTub), fuzzy onions (Fzo), protamine A (ProtA), and spermatocyte arrest (Sa) and homologs, orthologs, and paralogs thereof. In some embodiments, the nucleic acid sequence construct includes one or more second guide polynucleotides targeting one or more male-specific genomic sequence required for male fertility. The functional conservation of [[p]]βTubulin 85D including Anopheles and Aedes aegypti is described in Catteruccia et al., Nat. Biotechnol. 23, 1414-141 7 (2005) and Smith et al., Insect Mol. Biol. 16, 61-71 (2007), the entire contents of both of which are incorporated herein by reference. Also, see, for example, International Published Application No. WO 2019/103982.

In some embodiments, the gRNA is disclosed herein, such as in Table 1. In further embodiments, the gRNA is available to one of skill in the art, see for example, U.S. Patent Application No. 2020/0367479, U.S. Patent Application No. 2020/0404892, U.S. Patent Application No. 2020/0270634, International Published Application Nos. WO 2021/016600, WO 2020/160150, WO 2021/016600, Kandul et al. Nat Commun. 2020 Apr. 30; 11(1):2106, or Kandul et al. Nat Commun. 2019 Jan. 8; 10(1):84.

In some embodiments, the gRNA targets GATTGTCAACTACTTGCCCC (SEQ ID NO. 3). In further embodiments, the gRNA comprises, or consists essentially of, or yet further consists of a polynucleotide complementary to GATTGTCAACTACTTGCCCC (SEQ ID NO. 3). In yet further embodiments, the gRNA comprises, or consists essentially of, or yet further consists of GGGGCAAGTAGTTGACAATC (SEQ ID NO: 4). In yet further embodiments, the gRNA comprises, or consists essentially of, or yet further consists of GGGGCAAGUAGUUGACAAUC (SEQ ID NO: 5).

In some embodiments, the gRNA targets CGGCGAGAAAGAGAATACCA (SEQ ID NO: 6). In further embodiments, the gRNA comprises, or consists essentially of, or yet further consists of a polynucleotide complementary to CGGCGAGAAAGAGAATACCA (SEQ ID NO: 6). In yet further embodiments, the Grna comprises, or consists essentially of, or yet further consists of TGGTATTCTCTTTCTCGCCG (SEQ ID NO: 7). In yet further embodiments, the gRNA comprises, or consists essentially of, or yet further consists of UGGUAUUCUCUUUCUCGCCG (SEQ ID NO: 8).

In some embodiments, the gRNA targets GATTCCGTACTTTGCAGACG (SEQ ID NO: 9). In further embodiments, the gRNA comprises, or consists essentially of, or yet further consists of a polynucleotide complementary to GATTCCGTACTTTGCAGACG (SEQ ID NO: 9). In yet further embodiments, the gRNA comprises, or consists essentially of, or yet further consists of CGTCTGCAAAGTACGGAATC (SEQ ID NO: 10). In yet further embodiments, the gRNA comprises, or consists essentially of, or yet further consists of CGUCUGCAAAGUACGGAAUC (SEQ ID NO: 11).

In some embodiments, the gRNA targets CCTGAGTGTGCATCAGCTGG (SEQ ID NO: 12). In further embodiments, the gRNA comprises, or consists essentially of, or yet further consists of a polynucleotide complementary to CCTGAGTGTGCATCAGCTGG (SEQ ID NO: 12). In yet further embodiments, the gRNA comprises, or consists essentially of, or yet further consists of CCAGCTGATGCACACTCAGG (SEQ ID NO: 13). In yet further embodiments, the gRNA comprises, or consists essentially of, or yet further consists of CCAGCUGAUGCACACUCAGG (SEQ ID NO: 14).

In further embodiments, the gRNA comprises, or consists essentially of, or yet further consists of a sequence complementary to the target sequence and a gRNA scaffold. As used herein, a gRNA scaffold serves as a binding scaffold for the Cas nuclease. In some embodiments, the gRNA scaffold comprises, or consists essentially of, or yet further consists of a nucleotide sequence encoded by gttttagagctagaaatagcaagttaaaataaggctagtccgttatcaacttgaaaaagtggcaccgagtcggtgc (SEQ ID NO: 15). In further embodiments, the gRNA scaffold comprises, or consists essentially of, or yet further consists of gcaccgacucggugccacuuuuucaaguugauaacggacuagccuuauuuuaacuugcuauuucuagcucuaaaac (SEQ ID NO: 16).

In some embodiments, the gene editing system, the isolated or engineered polynucleotide, or the vector as disclosed herein comprises a polynucleotide encoding one or more of the gRNAs.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

In some embodiments of the present disclosure, the genetically modified insects and methods for generating the genetically modified insects include insects from the Order Diptera, Lepidoptera, or Coleoptera. In some embodiments of the present disclosure, the genetically modified insects and methods for generating the genetically modified insects include an insect selected from a mosquito of the genera Stegomyia, Aedes, Anopheles, or Culex. Of these genera, example mosquito species include Aedes aegypti, Aedes albopictus, Ochlerotatus triseriatus (Aedes triseriatus), Anopheles stephensi, Anopheles albimanus, Anopheles gambiae, Anopheles quadrimaculatus, Anopheles freeborni, Culex species, or Culiseta melanura.

In some embodiments, the insect as disclosed herein is an embryo, a larvae, or an adult. In further embodiments, the insect as disclosed herein is a 1^{st} instar larval. In yet further embodiments, the insect as disclosed herein is a 2^{nd} instar larval.

In some embodiments, the term “insect” refers to mosquitoes, ticks, flies, ants and cockroaches and other insects, nematodes, that cause annoyance or injurious to animals, or plants or humans. In some embodiments, insects refer to a class (Insecta) of arthropods (such as bugs or bees) with well-defined head, thorax, and abdomen, only three pairs of legs, and typically one or two pairs of wings.

In some embodiments, the insect can be in any stage of the life cycle, such as egg or embryo, larva (1^{st} instar, 2^{nd} instar, 3^{rd} instar, pre-pupa), pupa, or adult. In some embodiments, insect as used herein also refers to insect eggs or embryos. In some embodiments, insect as used exclude insect egg. In some embodiments, insects refers to embryo, larvae, pupa or adult. In further embodiments, insects refers to embryo, larvae, or adult. In yet further embodiments, insects refers to larvae or adult or both.

As used herein, a larva is the juvenile form of an insect, which often has a different appearance to the adult and can possess bodily organs that the adult inset does not possess (and vice versa). An instar is a developmental stage of arthropods, such as insects, between each moult (ecdysis), until sexual maturity is reached. Arthropods must shed the exoskeleton in order to grow or assume a new form. Differences between instars can often be seen in altered body proportions, colors, patterns, changes in the number of body segments or head width. After moulting, i.e. shedding their exoskeleton, the juvenile arthropods continue in their life cycle until they either pupate or moult again. The instar period of growth is fixed; however, in some insects, like the Salvinia stem-borer moth, the number of instars depends on early larval nutrition.

In some embodiments where the insect is a Drosophila, the insect can be in any one of the following developmental stages: embryogenesis, which is a fast process completed 24 h after fertilization of the oocyte by the male sperm; larval stage, which lasts about 4 days, including 3 instars separated by molting transitions; pupal stage, which is after encapsulation of the 3d instar larva, starts and lasts around 4 days; and adult life having a lifespan of around 30 days.

In some embodiments, the genetically modified insects and methods for generating the genetically modified insects, or any other embodiments and aspects of the disclosure include any insect selected from one of the following: tephritid fruit fly selected from Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata), Caribbean fruit fly (Anastrepha suspensa), Oriental Fruit Fly (Bactrocera dorsalis), West Indian fruit fly (Anastrepha obliqua), the New World screwworm (Cochliomyia hominivorax), the Old World screwworm (Chrysomya bezziana), Australian sheep blowfly/greenbottle fly (Lucilia cuprina), the pink bollworm (Pectinophora gossypiella), the European Gypsy moth (Lymantria dispar), the Navel Orange Worm (Amyelois transitella), the Peach Twig Borer (Anarsia lineatella), the rice stem borer (Tryporyza incertulas), the noctuid moths, Heliothinae, the Japanese beetle (Papilla japonica), White-fringed beetle (Graphognatus spp.), Boll weevil (Anthonomous grandis), the Colorado potato beetle (Leptinotarsa decern lineata), the vine mealybug (Planococcus ficus), Asian citrus psyllid (Diaphorina citri), Spotted wing Drosophila (Drosophila suzukii), Bluegreen sharpshooter (Graphocephala atropunctata), Glassy winged sharpshooter (Homalodisca vitripennis), Light brown apple moth (Epiphyas postvittana), Bagrada bug (Bagrada hilaris), Brown marmorated stink bug (Halyomorpha halys), Asian Gypsy Moth selected from the group of Lymantria dispar asiatica, Lymantria dispar japonica, Lymantria albescens, Lymantria umbrosa, and Lymantria postalba, Asian longhorned beetle (Anoplophora glabripennis), Coconut Rhinoceros Beetle (Oryctes rhinoceros), Emerald Ash Borer (Agrilus planipennis), European Grapevine Moth (Lobesia botrana), European Gypsy Moth (Lymantria dispar), False Codling Moth (Thaumatotibia leucotreta), fire ants selected from Solenopsis invicta Buren, and S. richteri Forel, Old World Bollworm (Helicoverpa armigera), Spotted Lanternfly (Lycorma delicatula), Africanized honeybee (Apis mellifera scutellata), Fruit and shoot borer (Leucinodes orbonalis), corn root worm (Diabrotica spp.), Western corn rootworm (Diabrotica virgifera), Whitefly (Bemisia tabaci), House Fly (Musca Domestica), Green Bottle Fly (Lucilia cuprina), Silk Moth (Bombyx mori), Red Scale (Aonidiella aurantia), Dog heartworm (Dirofilaria immitis), Southern pine beetle (Dendroctonus frontalis), Avocado thrip (Thysanoptera Spp.), Botfly selected from Oestridae spp. and Dermatobia hominis), Horse Fly (Tabanus sulcifrons), Horn Fly (Haematobia irritans), Screwworm Fly selected from Cochliomyia macellaria (C. macellaria), C. hominivorax, C. aldrichi, or C. minima, Tsetse Fly (Glossina spp.), Warble Fly selected from Hypoderma bovis or Hypoderma lineatum, Spotted lanternfly (Lycorma delicatula), Khapra beetle (Trogoderma granarium), Honeybee mite (Varroa destructor), Termites (Coptotermes formosanus), Hemlock woolly adelgid (Adelges tsugae), Walnut twig beetle (Pityophthorus juglandis), European wood wasp (Sirex noctilio), Pink-spotted bollworm (pectinophora scutigera), Two spotted spider mite (Tertanychus urticae), Diamondback moth (Plutella xylostella), Taro caterpillar (Spodoptera litura), Red flour beetle (Tribolium castaneum), Green peach aphid (Myzus persicae), Cotton Aphid (Aphis gossypii), Brown planthopper (Nilaparvata lugens), Beet armyworm (Spodotera exigua), Western flower Thrips (Frankliniella occidentalis), Codling moth (Cydia pomonella), Cowpea weevil (Callosobruchus maculatus), Pea aphid (Acyrthosiphon pisum), Tomato leafminer (Tuta absoluta), Onion Thrips (Thrips tabaci), and Cotton bollworm (Helicoverpa armigera).

In one embodiment, the guide polynucleotide is a gRNA. The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature Biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

As used herein, Protospacer Adjacent Motif or PAM refers to a sequence adjacent to the target sequence that is necessary for Cas enzymes to bind target polynucleotide.

As used herein, the term “target” or “target sequence” refers to the section of the polynucleotide recognized by a CRISPR-guide complex. In some embodiments, the gRNA is complementary to the target sequence.

A “composition” is intended to mean a combination of active polypeptide, polynucleotide or antibody and another compound or composition, inert (e.g., a detectable label) or active (e.g., a gene delivery vehicle).

The term “protein,” “peptide,” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids that can comprise a protein's or peptide's sequence. As used herein the term “amino acid” refers to natural and/or unnatural or synthetic amino acids, including glycine and both the D and L optical isomers, amino acid analogs and peptidomimetics.

The term “isolated” as used herein refers to molecules or biologicals or cellular materials being substantially free from other materials.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose 6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as³² P, ³⁵ S or ¹²⁵ I.

As used herein, the term “purification marker” or “selectable marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “progeny” refers to a descendant or the descendants. In one embodiment, the progeny is an insect egg or a population thereof. In another embodiment, the progeny is an insect or a population thereof. In yet another embodiment, the progeny is one or more of the following: an insect egg, an insect, or a population thereof.

As used herein, the term “nuclear localization signal” or “NLS” refers to an amino acid sequence that ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. In some embodiment, the NLS is a nuclear localization signal of SV40 (simian virus40) large T antigen comprising, or consisting essentially of, or yet further consisting of PKKKRKV (SEQ ID NO: 17), optionally encoded by ccaaagaagaagcggaaggtc (SEQ ID NO: 18). In some embodiments, the NLS is a bipartite nuclear localization signal from nucleoplasmin comprising, or consisting essentially of, or yet further consisting of KRPAATKKAGQAKKKK (SEQ ID NO: 19), optionally encoded by aaaaggccggcggccacgaaaaaggccggccaggcaaaaaagaaaaag (SEQ ID NO: 20).

As used herein, the term “regulatory sequence” or “expression control sequence” or the like refers to a segment of a nucleic acid molecule which is capable of increasing or decreasing the expression of specific genes within an organism. Expression control or regulatory sequences may include, e.g., include appropriate transcription initiation, termination, promoter and enhancer sequences; efficient RNA processing signals such as splicing and polyadenylation (polyA) signals; sequences that stabilize cytoplasmic Mrna; sequences that enhance translation efficiency (i.e., Kozak consensus sequence); sequences that enhance protein stability; and when desired, sequences that enhance secretion of the encoded product. A promoter may be selected from amongst a constitutive promoter, a tissue-specific promoter, a cell-specific promoter, a promoter responsive to physiologic cues, or an inducible promoter.

Inducible promoters may be suitable for use in the disclosed invention, for example including promoters responsive to exogenous agents (e.g., pharmacological agents) or to physiological cues (such as temperature). These response elements include, but are not limited to a hypoxia response element (HRE) that binds HIF-Iα and β, a metal-ion response element such as described by Mayo et al. (Mayo et al, Cell 29:99-108 (1982)); Brinster et al. (Brinster et al. Nature 296:39-42 (1982)) and Searle et al. (Searle et al. Mol. Cell. Biol. 5:1480-1489 (1985)); or a heat shock response element such as described by Nouer et al. (Nouer et al. in: Heat Shock Response, ed. Nouer, L., CRC, Boca Raton, Fla., ppI67-220, 1991). In one embodiment, a regulatable promoter that provides tight control over the transcription of the polynucleotide, e.g., via a pharmacological agent, or transcription factors activated by a pharmacological agent or in alternative embodiments, physiological cues. In some embodiments, promoter that are non-leaky and that can be tightly controlled are used. In some embodiments, promoter that is leaky can be used. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and Rtta activated by tetracycline. In one aspect of the invention, the gene switch is an EcR-based gene switch. Examples of such systems include, without limitation, the systems described in U.S. Pat. Nos. 6,258,603, 7,045,315, U.S. Patent Application No. 2006/0014711, U.S. Patent Application No. 2007/0161086, and International Published Application No. WO 2001/70816. Examples of chimeric ecdysone receptor systems are described in U.S. Pat. No. 7,091,038, U.S. Patent Application No. 2002/0110861, U.S. Patent Application No. 2004/0033600, U.S. Patent Application No. 2004/0096942, U.S. Patent Application No. 2005/0266457, and U.S. Patent Application No. 2006/0100416, and International Published Application Nos. WO 2001/70816, WO 2002/066612, WO 2002/066613, WO 2002/066614, WO 2002/066615, WO 2002/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, Mass.).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the neutralizing antibody construct can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); the humanized tamoxifen-dep regulatable system (Roscilli et al., 2002, Mol. Ther. 6(5):653-63).

In some embodiments, the regulatory sequence comprises, or consists essentially of, or yet further consists of a temperature inducible promoter. In further embodiments, the promoter comprises, or consists essentially of, or yet further consists of an Hsp70Bb (Hsp70, Hsp, CG31359) promoter.

Heterozygous refers to the presence of unequal alleles at the corresponding chromosomal loci. Accordingly, the insect or insect egg or insect population or a progeny of each thereof can have a heterozygous copy of the polynucleotide or the system, i.e., the insect or insect egg or insect population or a progeny of each thereof only comprise one copy of the polynucleotide in the chromosomes.

The term homozygous means a genetic condition existing when identical alleles reside at corresponding loci on homologous chromosomes. Accordingly, the insect or insect egg or insect population or a progeny of each thereof can have a homozygous copy of the polynucleotide or the system, i.e., the insect or insect egg or insect population or a progeny of each thereof comprise two copies of the polynucleotide at corresponding loci on homologous chromosomes.

As used herein, an “RNA polymerase III promoter” (RNA Pol III or Pol III promoter) refers to a nucleotide sequence that directs the transcription of RNA by RNA polymerase III.

RNA polymerase III promoters may include a full-length promoter or a fragment thereof sufficient to drive transcription by RNA polymerase III. For a more detailed description of RNA polymerase III promoter types, structural features, and interactions with RNA polymerase III, as well as suitable RNA polymerase III promoters, see Schramm, L. and Hernandez, N. (2002) Genes Dev. 16:2593-620. Additional suitable Pol III promoters can be found, for example, at Gao et al. Mol Ther Nucleic Acids. 2018 Sep. 7; 12:135-145; or www.ebi.ac.uk/QuickGO/term/GO:0006383, last accessed on May 24, 2021.

In some embodiments, the polynucleotide as disclosed herein comprises, or consists essentially of, or yet further consists of an RNA polymerase III promoter, a guide RNA or a sequence complementary to or encoding thereof, and an RNA Polymerase III terminator, optionally from 5′ to 3′.

As used herein, an “RNA polymerase III terminator” refers to any nucleotide sequence that is sufficient to terminate a transcript transcribed by RNA polymerase III. As used herein, and unless specified, an RNA polymerase III terminator may refer to the transcribed RNA sequence itself or the DNA sequence encoding it. Examples of RNA polymerase III terminators may include, without limitation, a string of uridine nucleotides of at least 5-6 bases in length (for more information on RNA polymerase III terminators, see Marck, C., et al. (2006) Nucleic Acids Res 34(6):1816-35). In some embodiments, the RNA polymerase III terminator comprises, or consists essentially of, or yet further consists of UUUUUUUTUUUUUU (SEQ ID NO: 21). In some embodiments, the terminator comprises, or consists essentially of, or yet further consists of TTTTTTTTTT (SEQ ID NO: 22) or UUUUUUUUUU (SEQ ID NO. 23).

In some embodiments, the polynucleotide or the regulatory sequence as disclosed herein further comprises a three prime untranslated region 3′ UTR. Generally, the term “3′-UTR” refers to a part of the artificial nucleic acid molecule, which is located 3′ (i.e. “downstream”) of an open reading frame and which is not translated into protein. Typically, a 3′-UTR is the part of an mRNA, which is located between the protein coding region (open reading frame (ORF) or coding sequence (CDS)) and the poly(N/A) sequence of the (m)RNA. In the context of the invention, a 3′-UTR of the artificial nucleic acid molecule may comprise more than one 3′-UTR elements, which may be of different origin, such as sequence elements derived from the 3′-UTR of several (unrelated) naturally occurring genes. Accordingly, the term 3′-UTR may also comprise elements, which are not encoded in the template, from which an RNA is transcribed, but which are added after transcription during maturation, e.g. a poly(N/A) sequence. A 3′-UTR of the mRNA is not translated into an amino acid sequence. The 3′-UTR sequence is generally encoded by the gene, which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′ capping, splicing the pre-mature mRNA to excize optional introns and modifications of the 3′-end, such as polynucleotidylation/polyadenylation of the 3′-end of the pre-mature mRNA and optional endo-/or exonuclease cleavages etc. In some embodiments, the 3′ UTR comprises, or consists essentially of, or yet further consists of

(SEQ ID NO: 24)
ccgacatatatccgaaataactgcttgttttttttttttaccattatt
accatcgtgtttactgtttattgccccctcaaaaagctaatgtaatta
tatttgtgccaataaaaacaagatatgacctatagaatacaagtattt
ccccttcgaacatccccacaagtagactttggatttgtcttctaacca
aaagacttacacacctgcataccttacatcaaaaactcgtttatcgct
acataaaacaccgggatatattttttatatacatacttttcaaatcgc
gcgccctcttcataattcacctccaccacaccacgtttcgtagttgct
ctttcgctgtctcccacccgctctccgcaacacattcaccttttgttc
gacgaccttggagcgactgtcgttagttccgcgcgattcggttcgctc
aaatggttccgagtggttcatttcgtctcaatagaaattagtaataaa
tatttgtatgtacaatttatttgctccaatatatttgtatatatttcc
ctcacagctatatttattctaatttaatattatgactttttaaggtaa
ttttttgtgacctgttcggagtgattagcgttacaatttgaactgaaa
gtgacatccagtgtttgttccttgtgtagatgcatctcaaaaaaatgg
tgggcataatagtgttgtttatatatatcaaaaataacaactataata
ataagaatacatttaatttagaaaatgcttggatttcactggaactag.

### Modes for Carrying Out the Disclosure

Releases of sterile males are the gold standard for many insect population control programs, and precise sex sorting to remove females prior to male releases is essential to the success of these operations. To advance traditional methods for scaling the generation of sterile males, Applicant previously described a CRISPR-mediated precision-guided sterile insect technique (pgSIT), in which Cas9 and gRNA strains were genetically crossed to generate sterile males for release. While effective at generating F₁ sterile males, pgSIT requires a genetic cross between the two parental strains which requires maintenance and sexing of two strains in a factory. Therefore, to further advance pgSIT by removing this crossing step, here Applicant provides a next-generation Temperature-Inducible pgSIT (TI-pgSIT) technology and demonstrate its proof-of-concept in Drosophila melanogaster.

Importantly, Applicant was able to develop a true-breeding strain for TI-pgSIT that eliminates the requirement for sex sorting, a feature that may help further automate production at scale.

Disclosed herein is a one-locus inducible precision guided Sterile Insect technique (pgSIT) which makes insect-sex sorting completely unnecessary, since only one transgenic insect line would be bred, instead of two lines. The chemical/antibiotic induced-expression of Cas9 is disclosed herein. However, without wishing to be bound by the theory, due to the possible effect of the chemical/antibiotic induced-expression on the fitness of generated pgSIT flies and its leakiness, a temperature-inducible promoter to induce the pgSIT phenotypes is also disclosed and utilized in the Examples. Also shown in the Examples are tests confirming the feasibility of this approach by using trans-heterozygous flies carrying the temperature-inducible Cas9 and dgRNAs transgenes. At working prototype stage, two functional transgenic lines were generated. Applicant further engineered the one-locus pgSIT genetic cassette, generated transgenic flies, and confirmed heat-shock can induce 100% penetrance of the pgSIT phenotypes in otherwise the pure-bred one-locus phSIT transgenic line(s), and thus, demonstrating the approach is working.

As detailed in the Examples, large numbers of transgenic insects (one transgenic line) are perpetually maintained at the permissive temperature of 18° C., in a factory. At any time, some part of the insects shifted to the restrictive temperature of 26° C., and batches of developing larvae are heat-shocked for one hour at 37° C., while parent insects passaged on new food to produce more larvae, and continue development at 26° C. Only sterile males are emerged after the development of heat-shocked larvae. All females perish during the development, and sterile males are ready for the releases into wild populations, where they will compete with wildtype males, mate with wildtype females, and cause no progeny from the mated females resulting in decreasing numbers of targeted insect species.

The non-limiting examples shown established the design to build genetic systems for the positive activation of any lethal transgene by temperature. This is especially useful for insect population control since the maintenance of large numbers of transgenic insects are required before the induction of a specific transgene activates the production of large numbers of sterile males ready for releases.

Briefly, the advantages of the invention as disclosed herein include but are not limited to:
1. It allows the pure breeding of a single transgenic line, instead of two transgenic lines. So, the costs related to maintenance of transgenic lines for insect population suppression (biocontrol) are reduced.
2. It makes sex-sorting obsolete. No more insect sexing for the genetic cross to generate sterile males for releases is required. Insect sex-sorting is laborious, costly, and time consuming. So, it saves money here too.
3. The temperature-activation does not affect the fitness of release insect males, as compared to the feeding these insects of chemicals or antibiotics. So, it does it without decreasing the fitness and competitiveness of release sterile males.

One exemplified example of using the invention as disclosed is biological control (population suppression) of insect carriers of diseases or agricultural pests.

### Compositions and Methods

Provided herein is a gene editing system comprising, or alternatively consisting essentially of, or yet further consisting of: (a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini; (b) a regulatory sequence directing the endonuclease expression in a cell, optionally wherein the cell is an insect germline cell, optionally wherein the regulatory sequence is temperature-sensitive, and further optionally wherein the regulatory sequence comprises or consists essentially of, or yet further consists of a heat-shock protein 70B (Hsp70Bb) promoter; (c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF); (d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell; (e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and (f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell.

In some embodiments, the U6.3 promoter comprises, or consists essentially of, or yet further consists of

(SEQ ID NO: 25)
gaattcttttttgctcacctgtgattgctcctactcaaatacaaaaac
atcaaattttctgtcaataaagcatatttatttatatttattttacag
gaaagaattccttttaaagtgtattttaacctataatgaaaaacgatt
aaaaaaaatacataaaataattcgaaaatttttgaatagcccaggttg
ataaaaattcatttcatacgttttataacttatgcccctaagtatttt
ttgaccatagtgtttcaattctacattaattttacagagtagaatgaa
acgccacctactcagccaagaggcgaaaaggttagctcgccaagcaga
gagggcgccagtgctcactactttttataattctcaacttctttttcc
agactcagttcgtatatatagacctattttcaatttaacgtcg.

10.1351 Also, provided herein is a gene editing system. The system comprises, or alternatively consists essentially of, or yet further consists of: (a) a polynucleotide encoding an endonuclease, (b) a regulatory sequence directing the endonuclease expression in a cell, (c) a guide polynucleotide targeting a female-essential genomic sequence that is required for female-specific viability, or a complementary sequence of the guide polynucleotide, or a polynucleotide expressing the guide polynucleotide, and (e) a guide polynucleotide targeting a male sterility genomic sequence that is required for male-specific fertility, or a complementary sequence of the guide polynucleotide, or a polynucleotide expressing the guide polynucleotide.

In some embodiments, the endonuclease comprises, or consists essentially of, or yet further consists of Cas9. In further embodiments, the Cas9 is a Streptococcus pyogenes Cas9 or a mutant thereof. Additionally or alternatively, the polynucleotide of (a) further encodes a nuclear localization signal at the amino terminus of the endonuclease or the carboxyl terminus of the endonuclease or both termini.

In some embodiments, the cell is an insect germline cell.

In some embodiments, the regulatory sequence of (b) is temperature-sensitive. In further embodiments, the regulatory sequence of (b) comprises, or consists essentially of, or yet further consists of a temperature inducible promoter. In yet further embodiments, the regulatory sequence of (b) comprises, or consists essentially of, or yet further consists of a heat-shock protein 70B (Hsp70Bb) promoter. In some embodiments, the Hsp70Bb promoter comprises, or consists essentially of, or yet further consists of

(SEQ ID NO: 26)
tcgagaaatttctctggccgttattcgttattctctcttttctttttg
ggtctctccctctctgcactaatgctctctcactctgtcacacagtaa
acggcatactgctctcgttggttcgagagagcgcgcctcgaatgttcg
cgaaaagagcgccggagtataaatagaggcgcttcgtctacggagcga
caattcaattcaaacaagcaaagtgaacacgtcgctaagcgaaagcta
agcaaataaacaagcgcagctgaacaagctaaacaatctgcagtaaag
tgcaagttaaagtgaatcaattaaaagtaaccagcaaccaagtaaatc
aactgcaactactgaaatctgccaagaagtaattattgaatacaagaa
gagaactctg.

In some embodiments, the female-essential genomic sequence comprises, or consists essentially of, or yet further consists of a sex-specifically alternatively spliced sex-determination gene. In further embodiments, the female-essential genomic sequence comprises, or consists essentially of, or yet further consists of one or more of: sex lethal (Sxl), transformer (tra), or doublesex (dsxF).

In some embodiments, the system further comprises a regulatory sequence directing expression of the guide polynucleotide of (c) in a cell. In further embodiments, the regulatory sequence comprises, or consists essentially of, or yet further consists of a RNA pol III promoter. In yet further embodiments, the RNA pol III promoter is selected from the group consisting of H1, U6, or U6.3. In some embodiments, the cell is an insect germline cell.

In some embodiments, the male sterility genomic sequence comprises, or consists essentially of, or yet further consists of a gene active during spermatogenesis. In yet further embodiments, the male sterility genomic sequence comprises, or consists essentially of, or yet further consists of one or more of: βTubulin 85D (βTub), fuzzy onions (fzo), protamine A (ProtA), or spermatocyte arrest.

In some embodiments, the system further comprises a regulatory sequence directing expression of the guide polynucleotide of (e) in a cell. In further embodiments, the regulatory sequence comprises, or consists essentially of, or yet further consists of a RNA pol III promoter. In yet further embodiments, the RNA pol III promoter is selected from the group consisting of H1, U6, or U6.3. In some embodiments, the cell is an insect germline cell.

In some embodiments, the insect is selected from Drosophila melanogaster, Aedes aegypti, Aedes albopictus, Ceratitis capitate, or Drosophila suzukii.

In one embodiment, the cell produces the polynucleotide(s) and/or the vector(s). Additionally or alternatively, the cell is an insect cell. In one embodiment, the host cell is selected from an egg, a sperm, a zygote, or a germline cell.

In a further aspect, one or more of the following is provided: a genetically modified insect egg or a progeny thereof, a genetically modified insect or a progeny thereof, or an insect population comprising, or consisting essentially of, or yet further consisting of at least one genetically modified insect or a progeny thereof, comprising the gene editing system as disclosed herein.

In some embodiments, the insect egg or the insect comprises or consists essentially of, or yet further consists of one or two or more copies of any of (c)-(f). In some embodiments, the insect egg or the insect comprises or consists essentially of, or yet further consists of one or two or more copies of any of (a)-(f).

In some embodiments, the insect egg or the insect comprises a contiguous polynucleotide comprising any two, any three, any four, any five, or all of (a)-(f). In one embodiment, the contiguous polynucleotide further comprises a detectable or selectable marker or a polynucleotide encoding a detectable or selectable marker. Additionally or alternatively, the contiguous polynucleotide further comprises a sequence encoding a self-cleaving peptide between the polynucleotide of (a) and the polynucleotide encoding a detectable or selectable marker. In another embodiment, the contiguous polynucleotide further comprises a polyA sequence.

In some embodiments, the insect egg or the insect comprises a polynucleotide of pgSIT^{sxl,βTb, Hsp70Bb-Cas9} or a polynucleotide of pgSIT^{traB,βTub, Hsp70Bb-Cas9} as disclosed. In some embodiments, the polynucleotide comprises, or consists essentially of, or yet further consists of a Drosophila heat-shock protein 70B (Hsp70Bb) promoter directing the temperature-inducible expression of Cas9, a Cas9 coding sequence (such as a coding sequence of the Streptococcus pyogenes-derived Cas9), and a coding sequence of nuclear localization signals (NLS). In some embodiments, the NLS coding sequence is located at the 5′ end of the Cas9 coding sequence. Additionally or alternatively, the NLS coding sequence is located at the 3′ end of the Cas9 coding sequence. In further embodiments, the polynucleotide further comprises a coding sequence of a self-cleaving T2A peptide and a coding sequence of GFP or another detectable marker, serving as a visual indicator of Cas9 expression. In some embodiments, the Opie2-dsRed-SV40 marker transgene was included in the polynucleotide.

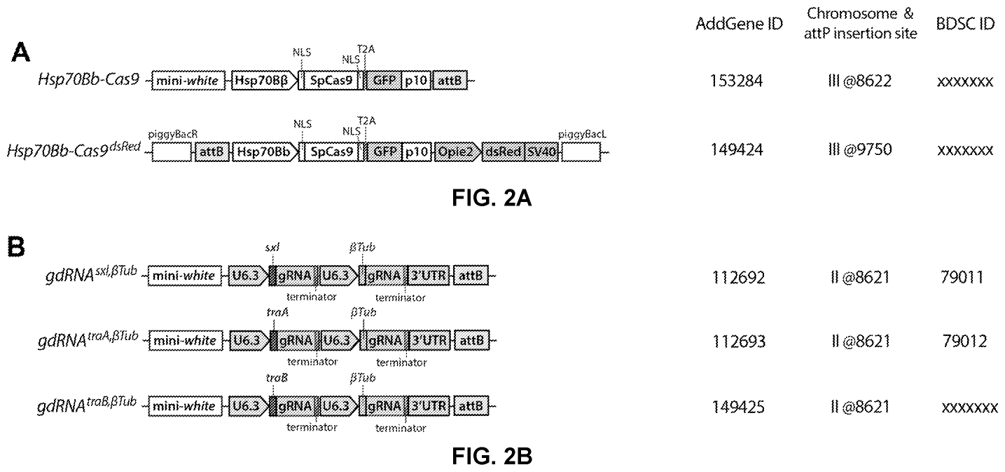

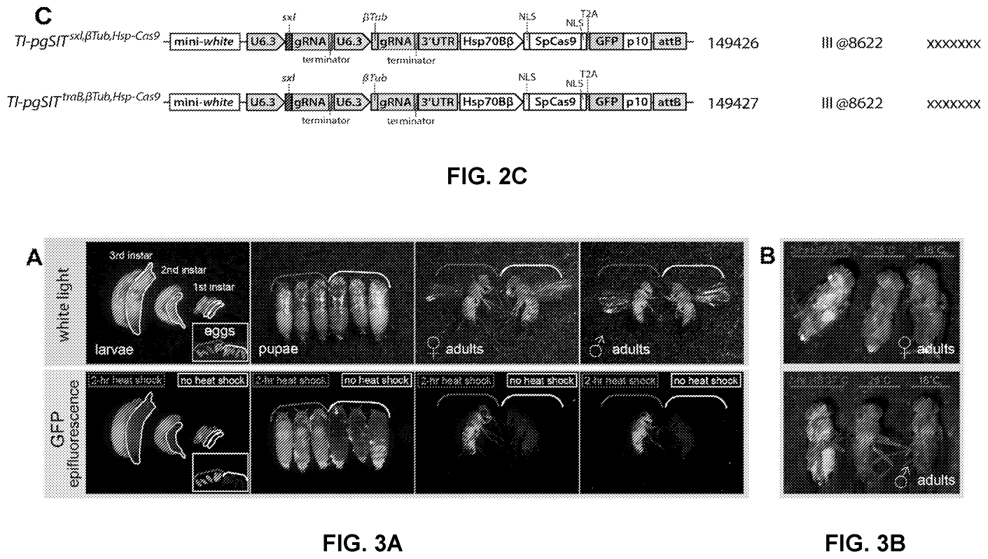

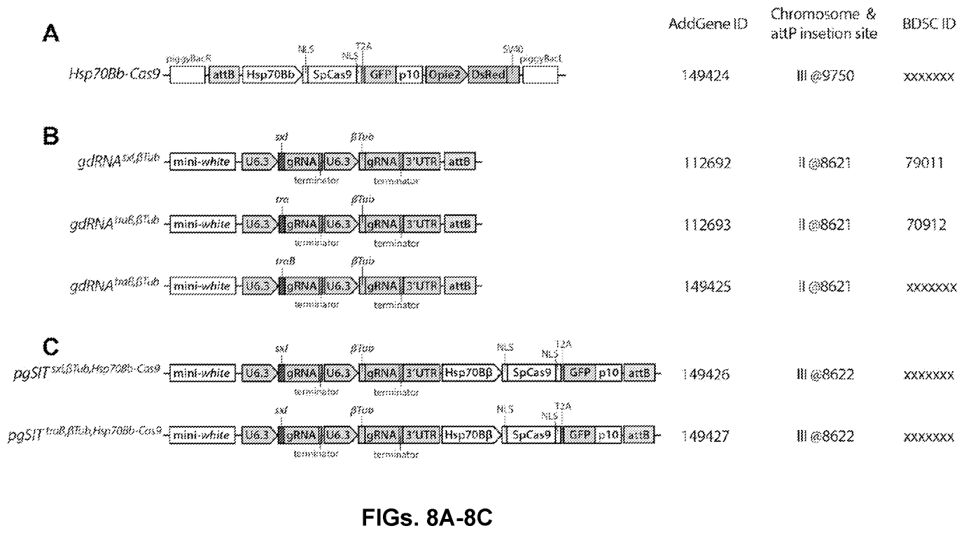

See, for example, FIGS. 2A and 8A. In some embodiments, the polynucleotide further comprises a double guide RNA (dgRNA) genetic construct. In some embodiments, the dgRNA genetic construct comprises, or consists essentially of, or yet further consists of a gRNA targeting βTubulin 85D (βTub) and a gRNA targeting sex lethal (sxl) or transformer (tra). In further embodiments, the dgRNA genetic construct further comprises a promoter, such as a Drosophila U6.3 promoter, directing the expression of the gRNAs. In yet further embodiments, the dgRNA construct is tracked by the mini-white marker gene. See, for example, FIGS. 2B and 8B. The gRNA sequences are indicated in the Table 1.

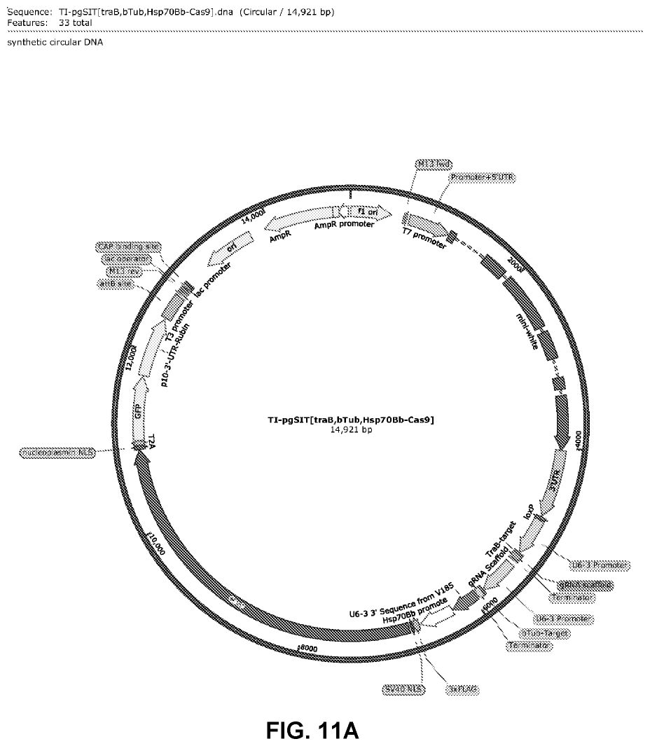

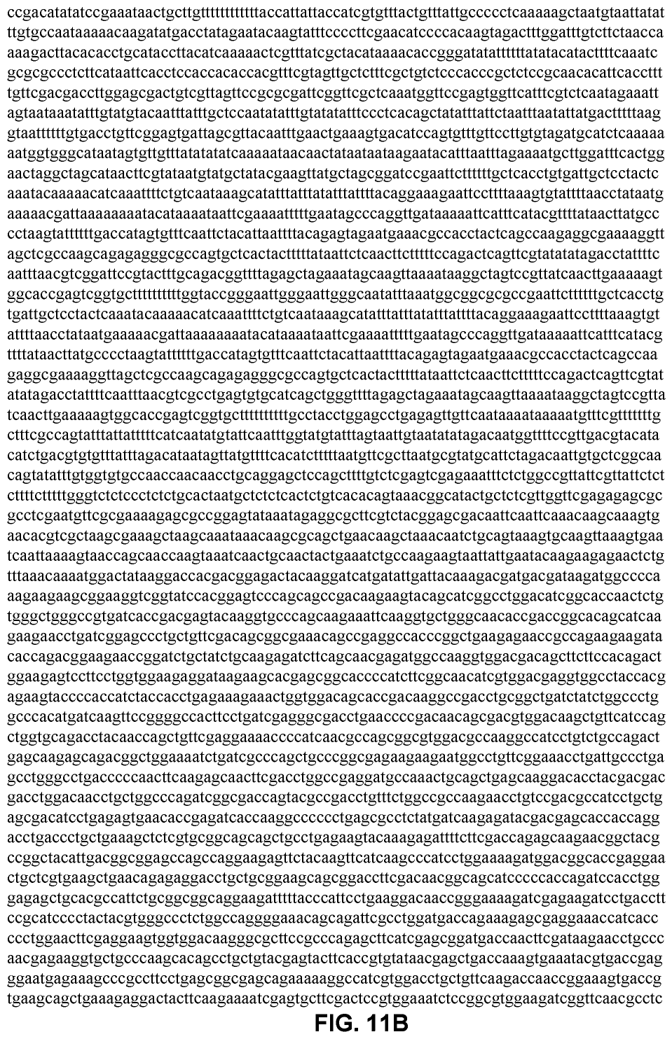

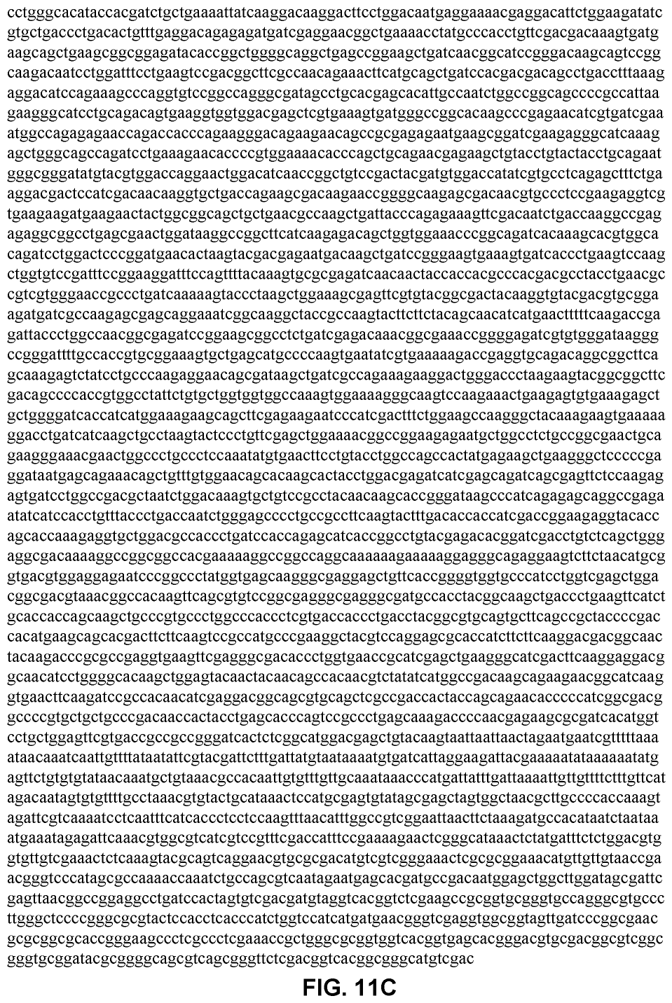

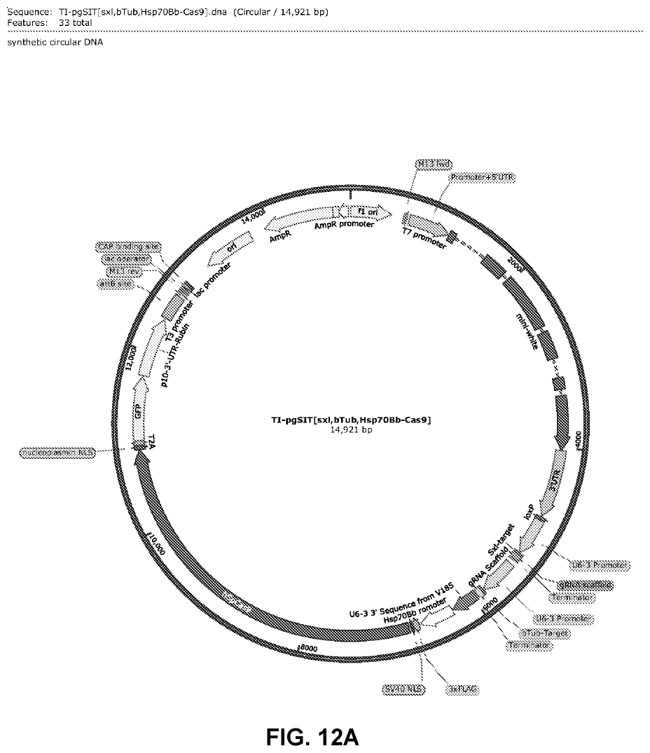

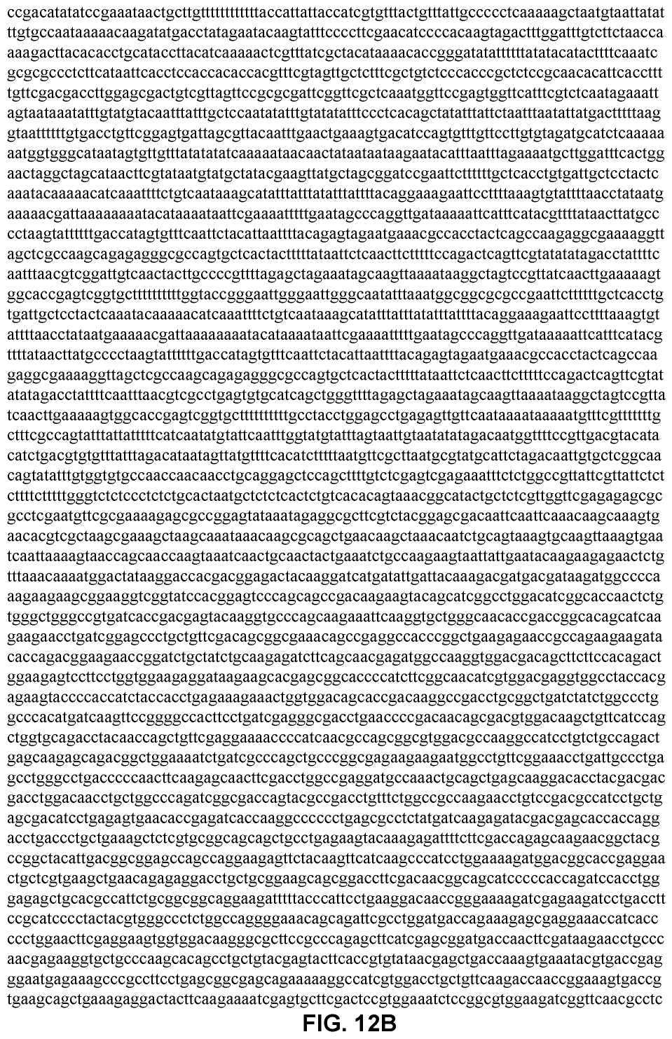

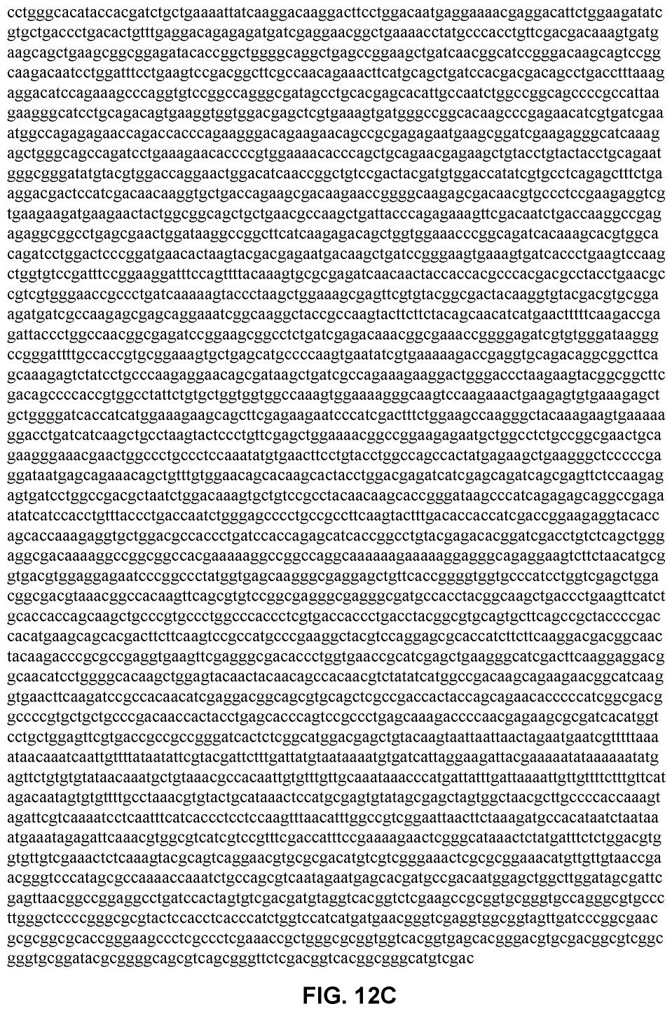

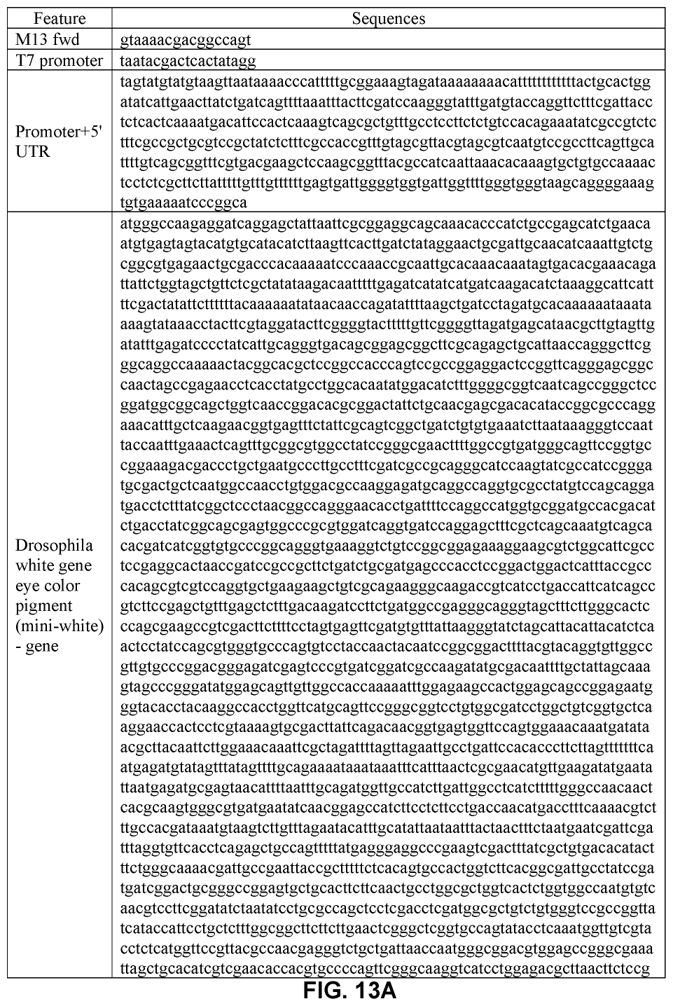

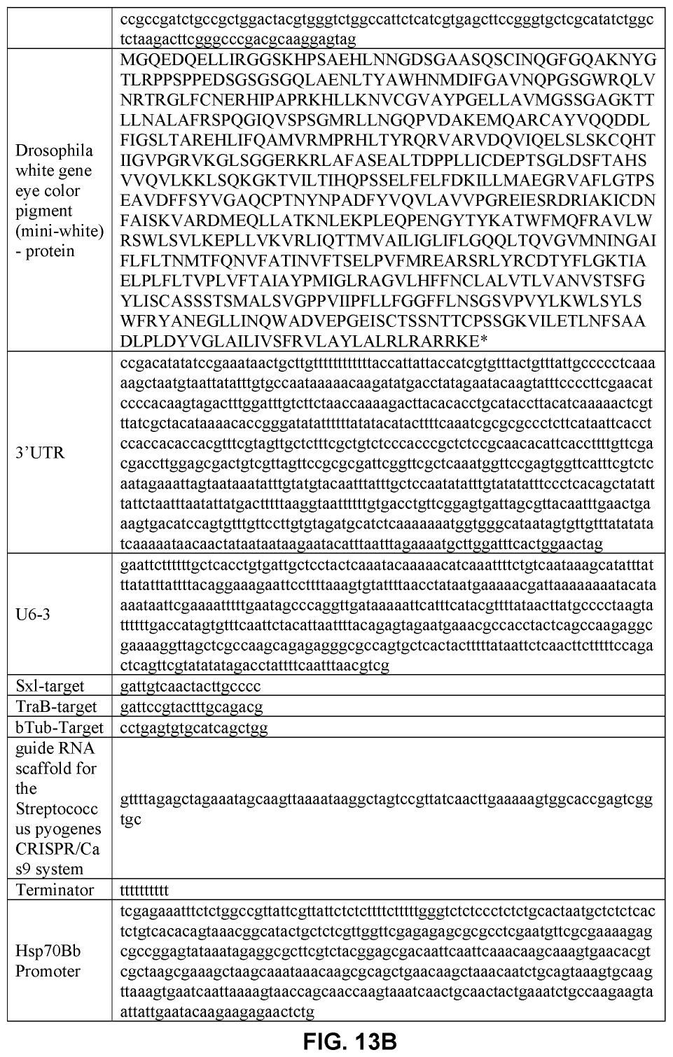

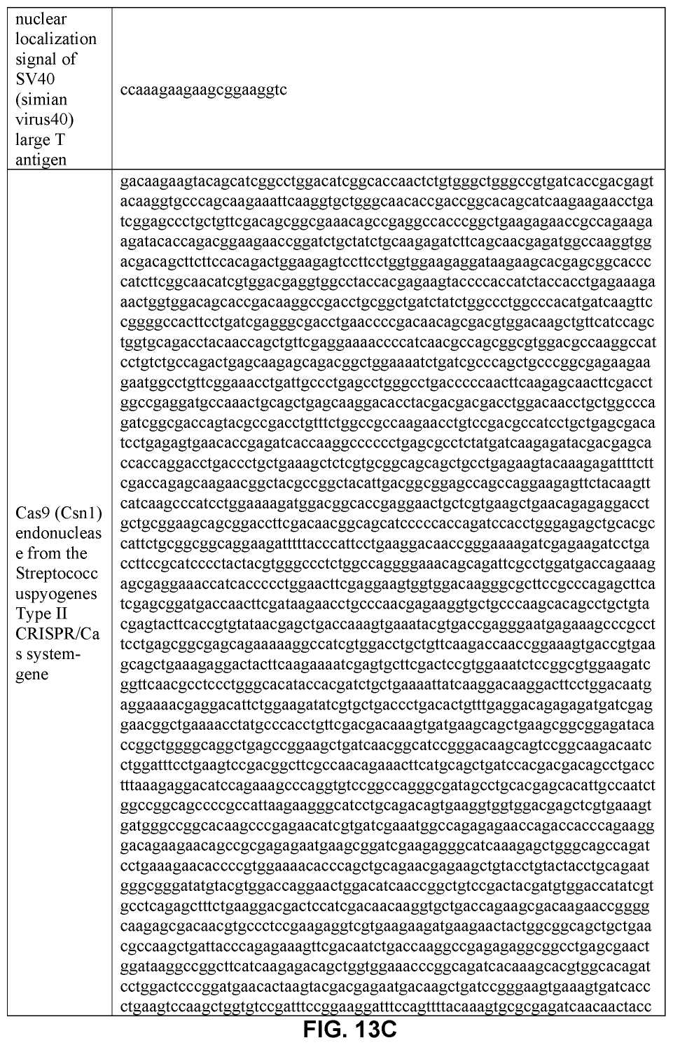

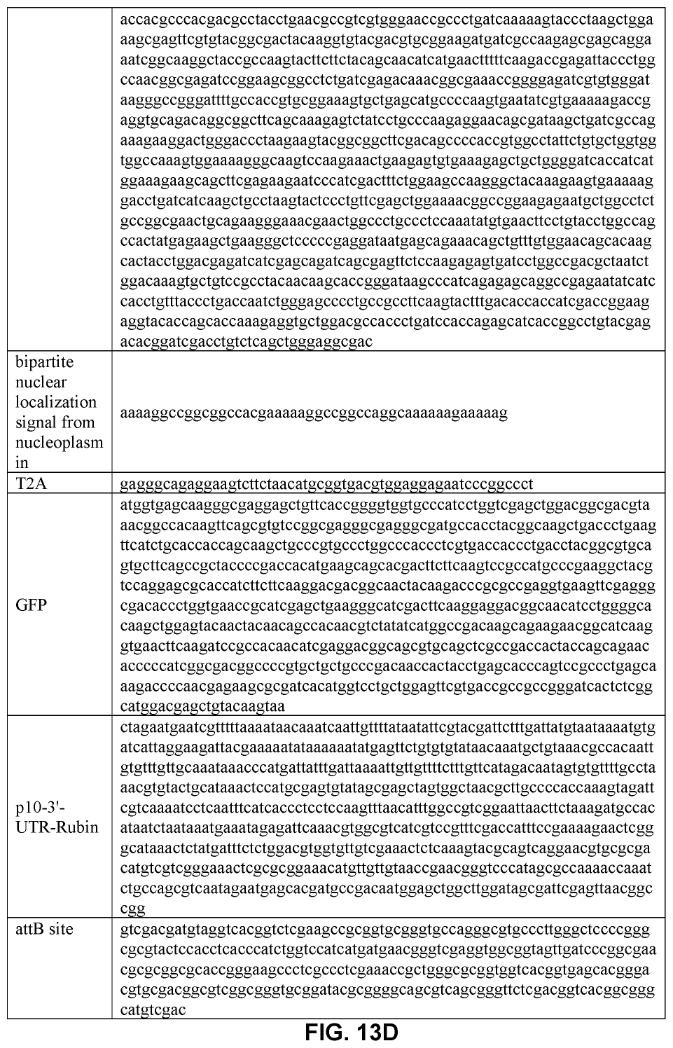

In some embodiments, the polynucleotide comprises, or consists essentially of, or yet further consists of a temperature-inducible precision guided Sterile Insect Technique (TI-pgSIT) genetic cassette. See, for example, FIGS. 2C and 8C. In some embodiments, the Hsp-70Bb-Cas9-T2A-GFP-p10 fragment was added to the two dgRNA constructs to build two TI-pgSIT cassettes. In some embodiments, the polynucleotide comprises, or consists essentially of, or yet further consists of a nucleic acid molecule as shown in FIGS. 11-13 or a fragment thereof.

In some embodiments, the genetic cassettes or the polynucleotides were site-specifically integrated at the P{CaryP}attP2 site on the 3rd chromosome (BDSC #8622).

In some embodiments, (a)-(f) are engineered to one or more of the chromosome(s) or chromosome site(s) of the insect egg or the insect. In one embodiment, the insect egg or the insect comprises homozygous (a)-(f). In another embodiment, the insect egg or the insect comprises a heterozygous (a)-(f).

In some embodiments, expression of the endonuclease is not activated and the insect egg or the insect or the insect population or a progeny of each thereof of is kept under a permissive temperature of the regulatory sequence of (b). 10.1.531 In one embodiment, the permissive temperature is about 18° C. or lower. In one embodiment, the permissive temperature is lower than about 26° C., such as about 25° C. or lower, about 24° C. or lower, about 23° C. or lower, about 22° C. or lower, about 21° C. or lower, about 20° C. or lower, about 19° C. or lower, about 18° C. or lower, about 17° C. or lower, about 16° C. or lower, about 15° C. or lower, about 14° C. or lower, about 13° C. or lower, about 12° C. or lower, about 11° C. or lower, about 10° C. or lower, about 9° C. or lower, about 8° C. or lower, about 7° C. or lower, about 6° C. or lower, about 5° C. or lower, about 4° C. or lower, about 3° C. or lower, about 2° C. or lower, about 1° C. or lower, about 0° C. or lower, or about 25° C. or lower. In some embodiments, the insect can grow or develop (such as from embryo to larvae, or from larvae to adult) under the permissible temperature.

Additionally or alternatively, the temperature inducible promoter in directing the expression of the endonuclease is not activated. In further embodiments, the temperature inducible promoter does not direct expression of the endonuclease under the permissible temperature. In yet further embodiments, the temperature inducible promoter does not direct expression of the endonuclease under the permissible temperature at a level sufficient to substantially reduce the function of the female-essential genomic sequence or the male sterility genomic sequence or both in the insect or insect egg.

In yet another embodiment, the progeny is the first, second, third, fourth, fifth, sixth, seventh, eighth, ninth, tenth, or more generations of the insect egg or insect or insect population.

In some embodiments, the insect is an embryo. In some embodiment, the insect is a larvae. In some embodiments, the insect is an adult.

In some embodiments, expression of the endonuclease is activated by keeping the insect egg, the insect, the population, or a progeny of each thereof under a restrictive temperature of the regulatory sequence of (b).

In one embodiment, the restrictive temperature is about 26° C. or higher. In one embodiment, the restrictive temperature is about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, about 40° C. or higher, about 41° C. or higher, about 42° C. or higher, about 43° C. or higher, about 44° C. or higher, about 45° C. or higher, about 46° C. or higher, about 47° C. or higher, about 48° C. or higher, about 49° C. or higher, or about 50° C. or higher. In some embodiments, the insect can grow or develop to, or is an adult having a fitness or mating competitiveness substantially similar to a wild type under the restrictive temperature. In further embodiments, the temperature inducible promoter directs expression of the endonuclease under the restrictive temperature.

In yet further embodiments, the temperature inducible promoter directs expression of the endonuclease under the restrictive temperature at a level sufficient to substantially reduce the function of the female-essential genomic sequence or the male sterility genomic sequence or both in the insect or insect egg.

Additionally or alternatively, the expression of the endonuclease is activated by one or more of heat-shock(s) of the insect, insect egg, insect population, or a progeny of each thereof. In some embodiments, a heat shock refers to keeping or incubating an insect or an insect egg at a temperature higher than its permissive temperature or its restrictive temperature. In one embodiment, the heat-shock is at about 37° C. In one embodiment, the heat-shock is at about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, about 40° C. or higher, about 41° C. or higher, about 42° C. or higher, about 43° C. or higher, about 44° C. or higher, about 45° C. or higher, about 46° C. or higher, about 47° C. or higher, about 48° C. or higher, about 49° C. or higher, or about 50° C. or higher.

Additionally or alternatively, the heat-shock is about 1 hour long, or about 2 hours long, or about 3 hours long, or about 4 hours long, or about 5 hours long, or about 6 hours long, or about 7 hours long, or about 8 hours long, or about 9 hours long, or about 10 hours long, or longer.

In some embodiments, the insect can grow or develop to, or is an adult having a fitness or mating competitiveness substantially similar to a wild type after the heat shock.

In some embodiments, the heat shock was performed on an insect egg, for example, on the 1^{st} day post oviposition, or on the 2nd day post oviposition, on the 3rd day post oviposition, on the 4th day post oviposition, on the 5th day post oviposition, on the 6th day post oviposition, within 1 week of oviposition, or longer.

In some embodiments, the heat shock was performed on an insect, such as an insect embryo, an insect larvae, or an insect adult. In some embodiments, the heat shock was performed in the 1^{st} instar larval stage. In some embodiments, the heat shock was performed in the 2^{nd} instar larvae.

In further embodiments, the temperature inducible promoter directs expression of the endonuclease during the heat shock. In yet further embodiments, the temperature inducible promoter directs expression of the endonuclease during the heat shock at a level sufficient to substantially reduce the function of the female-essential genomic sequence or the male sterility genomic sequence or both in the insect or insect egg. In some embodiments, the temperature inducible promoter directs expression of the endonuclease during the heat shock at a level of at least about 2 times of the one without the heat shock or higher, such as at least about 5 times, at least about 10 times, at least about 20 times, at least about 30 times, at least about 40 times, at least about 50 times, at least about 60 times, at least about 70 times, at least about 80 times, at least about 90 times, at least about 100 times, at least about 200 times, at least about 300 times, at least about 400 times, at least about 500 times, at least about 600 times, at least about 700 times, at least about 800 times, at least about 900 times, at least about 1000 times, at least about 1500 times, or higher of the expression level without the heat shock. In some embodiments, the expression level without the heat shock is determined using an insect or an insect egg or a population under a restrictive temperature. In some embodiments, the expression level without the heat shock is determined using an insect or an insect egg or a population under a permissive temperature.

In some embodiments, the incubation at the restrictive temperature is prior to or after (or both, i.e., prior to and after) the culture at the heat-shock.

In some embodiments, provided is a progeny of the insect egg or the insect or the insect population or a progeny of each thereof of as disclosed.

In some embodiments, the insect egg or the insect or the insect population or a progeny of each thereof of, comprises or consists essentially of, or yet further consists of at least about 50%, or at least about 75%, or at least about 80%, or at least about 85%, or at least about 90%, or at least about 95%, or at least about 96%, or at least about 97%, or at least about 98%, or at least about 99%, or up to 100% of the fitness or the mating competitiveness of a wild type. The Fitness and the mating competitiveness can be measured by one of skill in the art, such as using a method as disclosed in Experiment No. 2.

In some embodiments, a progeny of the insect egg or the insect or the insect population or a progeny of each thereof as disclosed herein, comprises or consists essentially of, or yet further consists of at least about 80%, or at least about 85%, or at least about 90%, or at least about 95%, or at least about 96%, or at least about 97%, or at least about 98%, or at least about 99%, or up to 100% sterile male. In some embodiments, the progeny is substantially free of female, such as not comprising any female, or comprising less than 0.000001% female, or less than 0.00001% female, or less than 0.0001% female, or less than 0.001% female, or less than 0.01% female, or less than 0.1% female, or less than 1% female.

In further embodiments, the progeny is substantially free of fertile female, such as not comprising any fertile female, or comprising less than 0.000001% fertile female, or less than 0.00001% fertile female, or less than 0.0001% fertile female, or less than 0.001% fertile female, or less than 0.01% fertile female, or less than 0.1% fertile female, or less than 1% fertile female. In some embodiments, the progeny is substantially free of fertile male, such as not comprising any fertile male, or comprising less than 0.000001% fertile male, or less than 0.00001% fertile male, or less than 0.0001% fertile male, or less than 0.001% fertile male, or less than 0.01% fertile male, or less than 0.1% fertile male, or less than 1% fertile male. In some embodiments, the progeny is substantially free of intersex, such as not comprising any intersex, or comprising less than 0.000001% intersex, or less than 0.00001% intersex, or less than 0.0001% intersex, or less than 0.001% intersex, or less than 0.01% intersex, or less than 0.1% intersex, or less than 1% intersex. Alternatively, the progeny can comprise some intersex since intersex is sterile. In some embodiments, the progeny comprises about 1%, or about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 30%, about 40%, or about 50%, or about 60%, or about 70%, or about 80% intersex.

In yet another aspect, provided is an isolated or engineered polynucleotide comprising, or consisting essentially of, or yet further consisting of any two, any three, any four, any five, or all of the following:
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell.

In yet another aspect, provided is an isolated or engineered polynucleotide comprising, or consisting essentially of, or yet further consisting of any two, any three, any four, any five, or all of the following: (a) a polynucleotide encoding an endonuclease, (b) a regulatory sequence directing the endonuclease expression in a cell, (c) a guide polynucleotide targeting a female-essential genomic sequence that is required for female-specific viability, or a complementary sequence of the guide polynucleotide, or a polynucleotide expressing the guide polynucleotide, or (e) a guide polynucleotide targeting a male sterility genomic sequence that is required for male-specific fertility, or a complementary sequence of the guide polynucleotide, or a polynucleotide expressing the guide polynucleotide.

In some embodiments, the endonuclease comprises, or consists essentially of, or yet further consists of Cas9. In further embodiments, the Cas9 is a Streptococcus pyogenes Cas9 or a mutant thereof. Additionally or alternatively, the polynucleotide of (a) further encodes a nuclear localization signal at the amino terminus of the endonuclease or the carboxyl terminus of the endonuclease or both termini.

In some embodiments, the cell is an insect germline cell.

In some embodiments, the regulatory sequence of (b) is temperature-sensitive. In further embodiments, the regulatory sequence of (b) comprises, or consists essentially of, or yet further consists of a temperature inducible promoter. In yet further embodiments, the regulatory sequence of (b) comprises, or consists essentially of, or yet further consists of a heat-shock protein 70B (Hsp70Bb) promoter.

In some embodiments, the female-essential genomic sequence comprises, or consists essentially of, or yet further consists of a sex-specifically alternatively spliced sex-determination gene. In further embodiments, the female-essential genomic sequence comprises, or consists essentially of, or yet further consists of one or more of: sex lethal (Sxl), transformer (tra), or doublesex (dsxF).

In some embodiments, the system further comprises a regulatory sequence directing expression of the guide polynucleotide of (c) in a cell. In further embodiments, the regulatory sequence comprises, or consists essentially of, or yet further consists of a RNA pol III promoter. In yet further embodiments, the RNA pol III promoter is selected from the group consisting of H1, U6, or U6.3. In some embodiments, the cell is an insect germline cell.

In some embodiments, the male sterility genomic sequence comprises, or consists essentially of, or yet further consists of a gene active during spermatogenesis. In yet further embodiments, the male sterility genomic sequence comprises, or consists essentially of, or yet further consists of one or more of: βTubulin 85D (βTub), fuzzy onions (fzo), protamine A (ProtA), or spermatocyte arrest.

In some embodiments, the system further comprises a regulatory sequence directing expression of the guide polynucleotide of (e) in a cell. In further embodiments, the regulatory sequence comprises, or consists essentially of, or yet further consists of a RNA pol III promoter. In yet further embodiments, the RNA pol III promoter is selected from the group consisting of H1, U6, or U6.3. In some embodiments, the cell is an insect germline cell.

In some embodiments, the insect is selected from Drosophila melanogaster, Aedes aegypti, Aedes albopictus, Ceratitis capitate, or Drosophila suzukii.

In some embodiments, the isolated or engineered polynucleotide comprises or consists essentially of, or yet further consists of one or two or more copies of any of (c)-(f). In some embodiments, the isolated or engineered polynucleotide comprises or consists essentially of, or yet further consists of one or two or more copies of any of (a)-(f).

In some embodiments, the isolated or engineered polynucleotide further comprises one or more of the following: a polynucleotide encoding a detectable or selectable marker, a sequence encoding a self-cleaving peptide between the polynucleotide of (a) and the polynucleotide encoding a detectable or selectable marker, and a polyA sequence.

In some embodiments, the isolated or engineered polynucleotide comprises or consists essentially of, or yet further consists of a polynucleotide of pgSIT^{sxl,βTub, Hsp70Bb-Cas9} or a polynucleotide of pgSIT^{traB,βTub, Hsp70Bb-Cas9} or both. See, FIGS. 2 and 8.

In some embodiments, the polynucleotide comprises, or consists essentially of, or yet further consists of a Drosophila heat-shock protein 70B (Hsp70Bb) promoter directing the temperature-inducible expression of Cas9, a Cas9 coding sequence (such as a coding sequence of the Streptococcus pyogenes-derived Cas9), and a coding sequence of nuclear localization signals (NLS). In some embodiments, the NLS coding sequence is located at the 5′ end of the Cas9 coding sequence. Additionally or alternatively, the NLS coding sequence is located at the 3′ end of the Cas9 coding sequence. In further embodiments, the polynucleotide further comprises a coding sequence of a self-cleaving T2A peptide and a coding sequence of GFP or another detectable marker, serving as a visual indicator of Cas9 expression. In some embodiments, the Opie2-dsRed-SV40 marker transgene was included in the polynucleotide. See, for example, FIGS. 2A and 8A. In some embodiments, the polynucleotide further comprises a double guide RNA (dgRNA) genetic construct. In some embodiments, the dgRNA genetic construct comprises, or consists essentially of, or yet further consists of a gRNA targeting βTubulin 85D (βTub) and a gRNA targeting sex lethal (sxl) or transformer (tra). In further embodiments, the dgRNA genetic construct further comprises a promoter, such as a Drosophila U6.3 promoter, directing the expression of the gRNAs. In yet further embodiments, the dgRNA construct is tracked by the mini-white marker gene. See, for example, FIGS. 2B and 8B. The gRNA sequences are indicated in the Table 1.

In some embodiments, the polynucleotide comprises, or consists essentially of, or yet further consists of a temperature-inducible precision guided Sterile Insect Technique (TI-pgSIT) genetic cassette. See, for example, FIGS. 2C and 8C. In some embodiments, the Hsp-70Bb-Cas9-T2A-GFP-p10 fragment was added to the two dgRNA constructs to build two TI-pgSIT cassettes. In some embodiments, the polynucleotide comprises, or consists essentially of, or yet further consists of a nucleic acid molecule as shown in FIGS. 11-13 or a fragment thereof.

In some embodiments, the genetic cassettes or the polynucleotides were site-specifically integrated at the P{CaryP}attP2 site on the 3rd chromosome (BDSC #8622).

In one aspect, provided is a vector comprising, or alternatively consisting essentially of, or yet further consisting of one or more of the polynucleotide(s) as disclosed herein. Also provided is an isolated or engineered host cell comprising any one or more of the polynucleotides as disclosed herein and/or any one or more of the vectors as disclosed herein. In one embodiment, the host cell produces the polynucleotide(s) and/or the vector(s). Additionally or alternatively, the host cell is an insect cell. In one embodiment, the host cell is selected from an egg, a sperm, a zygote, or a germline cell.

In some embodiments, the polynucleotide is engineered to one or more of the chromosome(s) or chromosome sites of the host cell. In one embodiment, the host cell comprises homozygous polynucleotides as disclosed herein. In another embodiment, the host cell comprises a heterozygous polynucleotide as disclosed herein.

Additionally provided is a method of reducing a wild-type insect population. In one embodiment, the method comprises, or consists essentially of, or yet further consists of introducing an insect egg or an insect or an insect population or a progeny of each thereof as disclosed herein, or the progeny as disclosed herein, to the wild-type insect population. In some embodiments, the method further comprises producing the insect egg, or the insect, or the insect population or the progeny of each thereof by introducing the system, or the polynucleotide, or the vector as disclosed herein into insect egg, or the insect, or the insect population, or a progeny of each thereof. In further embodiments, the method further comprises keeping the insect egg, the insect or the insect population or a progeny of each thereof comprising the system or the polynucleotide or the vector under a restrictive temperature. In yet further embodiments, the method further comprises heat shock the insect egg, the insect or the insect population or a progeny of each thereof comprising the system or the polynucleotide or the vector.

In one aspect, provided is a method of producing an insect egg, or an insect, or an insect population or a progeny of each thereof. In some embodiments, the method comprises, or consists essentially of, or yet further consists of introducing the system, or the polynucleotide, or the vector as disclosed herein into an insect egg, or an insect, or a population, or a progeny of each thereof, optionally a wildtype insect egg, a wildtype insect, or a population of each thereof, or a progeny of each thereof. In some embodiments, the produced insect egg, or insect, or insect population or a progeny of each thereof is genetically modified. In further embodiments, the produced insect egg, or insect, or insect population, or progeny of each thereof is those as disclosed herein. In further embodiments, the method further comprises keeping the insect egg, the insect or the insect population or a progeny of each thereof comprising the system or the polynucleotide or the vector under a restrictive temperature. In yet further embodiments, the method further comprises heat shock the insect egg, the insect or the insect population or a progeny of each thereof comprising the system or the polynucleotide or the vector. In some embodiments, the incubation at the restrictive temperature is prior to or after (or both, i.e., prior to and after) the culture at the heat-shock.

In one aspect, provided is a method of producing a population of insects or insect eggs or a progeny thereof. In some embodiments, the population or a progeny thereof is substantially free of female. Additionally or alternatively the population or a progeny thereof is substantially free of fertile female. In some embodiments, the population or a progeny thereof is substantially sterile male. In some embodiments, the method comprises, or consists essentially of, or yet further consists of introducing the system, or the polynucleotide, or the vector as disclosed herein into the insect population, or a progeny thereof. In further embodiments, the method further comprises keeping the population or a progeny thereof comprising the system or the polynucleotide or the vector under a restrictive temperature. In yet further embodiments, the method further comprises heat shock the population or a progeny thereof comprising the system or the polynucleotide or the vector. In some embodiments, the incubation at the restrictive temperature is prior to or after (or both, i.e., prior to and after) the culture at the heat-shock.

In further embodiments, provided is a composition or a kit comprising, or consisting essentially of, or yet further consisting of one or more of: a system as disclosed herein, a polynucleotide as disclosed herein, a vector as disclosed herein, a host cell as disclosed herein, an insect as disclosed herein, an insect egg as disclosed herein, an insect population as disclosed herein, or an insect progeny as disclosed herein. In further embodiments, the composition or kit is suitable for use in a method as disclosed herein. In yet further embodiments, the composition further comprises a carrier, such as a preservative. In some embodiment, the kit further comprises instructions of use. In further embodiments, the kit further comprises food suitable for feed the insect. Other reagents, such as buffer to dilute the polynucleotide or the vector to suitable concentrations for use can also be included in the kit. 10.1901 The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### EXAMPLES

### Experiment No. 1—Temperature-Inducible Precision Guided Sterile Insect Technique

Many insect population control approaches require the generation and release of large numbers of sterile males into natural populations. This control strategy was first proposed in 1955, when Edward Knipling proposed releasing sterile males to suppress insect populations-coined the sterile insect technique (SIT) (Knipling et al., J. Econ. Entomol. 48, 459-462 (1955)). SIT has since been successfully implemented to suppress wild populations of a variety of insects (Springer, Dordrecht, 2005, Sterile Insect Technique: Principles and Practice in Area-Wide Integrated Pest Management; and Hendrichs et al., Sustainable Development International 2, 75-79 (2000)), such as in the eradication of the new world screw-worm fly, Cochliomyia hominivorax, in the U.S. and Mexico (Scott et al., Entomol. Exp. Appl. 164, 226-236 (2017)). Notwithstanding, Knipling's vision of sexing sterilized insects to remove females prior to release has been challenging to accomplish, even in the screw-worm example, which has limited the implementation of SIT to other insects.

Finding better ways to sex separate insects is necessary, as field trials and models illustrate that releasing only sterile males significantly improves the efficiency of population suppression and can significantly reduce production costs (Knipling et al, 1955; and Rendón et al, Journal of Economic Entomology vol. 97 1547-1553 (2004)). Furthermore, since females are often the sex that transmit pathogens (e.g. mosquitoes), a reliable sexing method to guarantee female elimination prior to release is highly desirable for the implementation of these programs. Other related methods of insect population control, such as Release of Insects carrying a Dominant Lethal (RIDL) (Thomas et al., Science 287, 2474-2476 (2000)) and the Wolbachia-mediated Incompatible Insect Technique (IIT) (Laven et al., Nature vol. 216 383-384 (1967); Xi et al., Science 310, 326-328 (2005); and Bourtzis et al., Acta Trop. 132, S150-S163 (2014)), also require precise sexing methods to avoid female releases. Notably, IIT programs are based on repeated releases of Wolbachia-infected males, which are incompatible with wild females that lack the specific Wolbachia strain. Even the accidental release of a small fraction of Wolbachia-infected fertile females could lead to the wide-scale spread of Wolbachia, which would immunize populations against the particular IIT program, underscoring the importance of effective sex separation. However, with a few species-specific exceptions (Meza et al., PLoS One 13, e0208880 (2018); and Crawford et al., Nat. Biotechnol. 38, 482-492 (2020)), insect sex-sorting can be time consuming, labor intensive, error-prone, and species-dependent (Papathanos et al., Malar. J. 8 Suppl 2, S5 (2009); Lutrat et al., Trends in Parasitology vol. 35 649-662 (2019); and Kandul et al., Nature Communications vol. 11 (2020)).

Applicant recently developed an alternative platform for the generation and sex separation of sterile males using the CRISPR-mediated precision guided SIT (pgSIT) technology (Kandul et al., Nat. Commun. 10, 84 (2019); and Li et al., Eliminating Mosquitoes with Precision Guided Sterile Males. doi:10.21203/rs.3.rs-367110/v1). This technology mechanistically relies on lethal/sterile mosaicism, mediated by the precision and accuracy of CRISPR, to simultaneously disrupt specific genes essential for female viability and male fertility during development, ensuring the exclusive production of sterile males. To generate pgSIT sterile males in this system, two homozygous strains are raised that harbor either Cas9 or guide RNAs (gRNAs), which are genetically crossed to produce F₁ sterile male progeny that can be deployed at any life stage for population suppression. To further advance this system and mitigate the need for the genetic cross, Applicant herein describes a next-generation Temperature-Inducible pgSIT (TI-pgSIT) technology and demonstrate its proof-of-concept in Drosophila melanogaster.

Temperature-Inducible Cas9 Activation

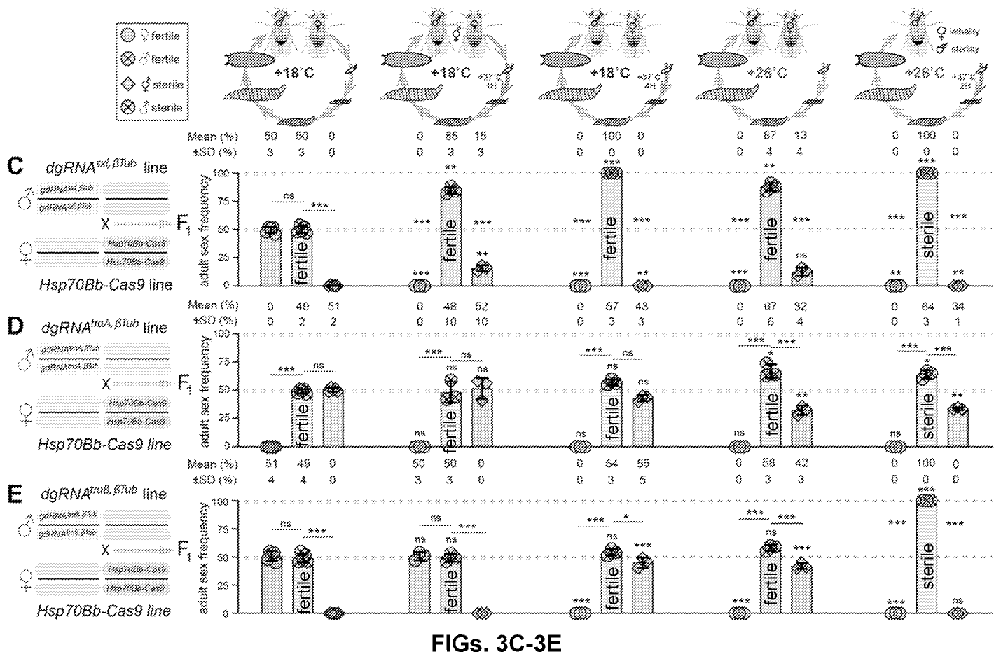

To generate an inducible platform that does not require exposure to radiation/chemicals/antibiotics, which can impact the fitness of released animals (Ballard & Melvin. Insect Molecular Biology vol. 16 799-802 (2007); Zeh, et al., Sci. Rep. 2, 375 (2012); Chatzispyrou et al., Cancer Res. 75, 4446-4449 (2015); Wang et al., Bioessays 37, 1045-1053 (2015) and Ourry et al., FEMS Microbiol. Ecol. 96, (2020)), Applicant utilized a temperature-inducible activation system. Applicant took advantage of the mechanism controlling the expression of Hsp70Bb, from the heat-shock 70 family of proteins, which can be temporarily activated by simply raising temperature to 37° C., a heat shock. When the temperature drops, the expression rapidly returns back to pre-shock levels (Spradling et al., J Mol. Biol. 109, 559-587 (1977); Ashburner & Bonner. Cell 17, 241-254 (1979); DiDomenico et al., Proc. Natl. Acad. Sci. U.S.A. 79, 6181-6185 (1982); Pelham & Bienz. The EMBO Journal vol. 1 1473-1477 (1982); Dreano et al., Gene 49, 1-8 (1986); and Petersen & Lindquist. Gene 72, 161-168 (1988)). Given this feature, Applicant leveraged the classical Hsp70Bb (Hsp70, Hsp, CG31359) promoter to generate a temperature-inducible Cas9 expression cassette (Hsp70Bb-Cas9) (FIG. 2A). For a visual indicator of promoter activity, Applicant also included a self-cleaving T2A peptide and eGFP coding sequence downstream (3′) from the Hsp-driven Cas9. With this, Applicant established a homozygous transgenic strain of Drosophila melanogaster. As the baseline expression of the Hsp70Bb promoter at 25° C. is well known (Steller & Pirrotta. EMBO J. 4, 3765-3772 (1985); Bishop & Corces. Genes Dev. 2, 567-577 (1988); and Bang & Posakony. Genes Dev. 6, 1752-1769 (1992)), Applicant compared expression at two temperatures—18° C. and 26° C. To visually assess the activity of Hsp70Bb-Cas9, Applicant compared GFP fluorescence in Hsp70Bb-Cas9 embryos, larvae, and adults, raised at either 18° C. or 26° C. with and without a two-hour heat shock at 37° C. (FIG. 3A). Without heat shock, Applicant did not detect visible changes in GFP fluorescence between flies raised at 18° C. and 26° C. (FIG. 3B). However, the heat-shocked individuals raised either at 18° C. or 26° C. had significantly brighter GFP fluorescence, indicating that exposure to 37° C. induces robust expression (FIG. 3B).

### Basal Expression of Cas9

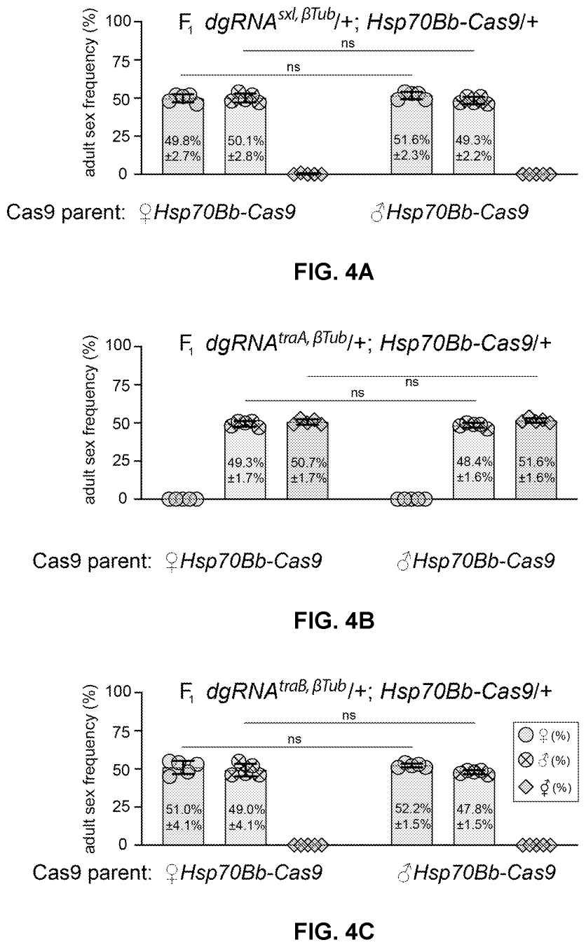

To genetically determine the basal activity of Hsp70Bb-Cas9 at 18° C., performed were a series of genetic crosses that would enable us to measure leaky expression. Applicant used constitutively expressing double gRNA (dgRNA) lines that target essential female viability genes, including sex-determination genes sex lethal (sxl) (Bell et al., Cell 65, 229-239 (1991)) or transformer (tra) (Boggs et al., Cell 50, 739-747 (1987)) in addition to an essential male fertility gene that is active during spermatogenesis, βTubulin 85D (βTub) (Kemphues et al., Cell 21, 445-451 (1980)). To target these genes, Applicant used previously generated lines (dgRNA^{sxl,βTub} and dgRNA^{traA,βTub}) (Kandul et al., 2019) and generated a new dgRNA line (dgRNA^{traB,βTub}) that targets a unique site in tra, each constitutively expressing two gRNAs: one targeting βTub and one targeting either sxl or tra (FIG. 2B, Table 1). Applicant crossed homozygous dgRNA males to homozygous Hsp70Bb-Cas9 females and raised the F₁ progeny at 18° C. The trans-heterozygous F₁ progeny harboring Hsp70Bb-Cas9 together with either dgRNA^{sxl,βTub} or dgRNA^{traB,βTub} developed into fertile females and males at equal frequencies: 49.8±2.7% ♀ vs 50.1±2.8 ♂ (p>0.884, a two-sided Student's t-test with equal variance; FIG. 3C, Table 2), and 51.0±4.1% ♀ vs 49.0±4.1 ♂ (p>0.452, a two-sided Student's t-test with equal variance; FIG. 3E, Table 2), respectively. Notably, the combination of paternal dgRNA^{traA,βTub} and maternal Hsp70Bb-Cas9 resulted in complete conversion of females into intersexes ( [symbol] ) (50.7±1.7% V vs 49.3±1.7 ♂, p>0.217, a two-sided Student's t-test with equal variance; FIG. 3D, Table 2) suggesting some degree of toxicity likely resulting from the leaky basal activity of Hsp70Bb-Cas9 combined with dgRNA^{traA,βTub}. To assess the fertility of the surviving F₁ progeny from these crosses, Applicant intercrossed F₁ flies and generated viable F₂ progeny at 18° C., except from intersex parents which were sterile. The reciprocal genetic cross of dgRTA females to Hsp70Bb-Cas9 males did not cause significant differences in the corresponding F₁ sex frequencies (FIG. 4, Table 2) suggesting that Hsp70Bb-Cas9 does not induce substantial maternal carryover of Cas9 protein at 18° C. Taken together, these results indicate that the Hsp70Bb promoter directed some leaky basal expression sufficient to convert females into intersexes when combined with dgRNA^{traA,βTub}, However F₁ trans-heterozygous flies (dgRNA^{sxl,βTub}/+; Hsp70Bb-Cas9/+ and dgRNA^{traB,βTub}/+; Hsp70Bb-Cas9/+) developed normally into fertile females and males.

TABLE 1
Target sequence of gRNA and
primers used in this disclosure.

| Name | Sequence (5′-3′) |
|---|---|
| gRNA^{sxl} | GATTGTCAACTACTTGCCCCAGG |
| | (SEQ ID NO: 27) |
| gRNA^{traA} | CGGCGAGAAAGAGAATACCATGG |
| | (SEQ ID NO: 28) |
| gRNA^{traB} | GATTCCGTACTTTGCAGACGAGG |
| | (SEQ ID NO: 29) |
| gRNA^{βTub} | CCTGAGTGTGCATCAGCTGGTGG |
| | (SEQ ID NO: 30) |
| 1137.C1F | GGTTCTCGACGGTCACGGCGGGCATGTCGACTCGA |
| | GAAATTTCTCTGGCCGTTATTCGTT |
| | (SEQ ID NO: 31) |
| 1137.C3R | CCGTCGTGGTCCTTATAGTCCATTTTGTTTAAACA |
| | GAGTTCTCTTCTTGTATTCAATAATTAC |
| | (SEQ ID NO: 32) |
| 2XgRNA-5F | TGTATGCTATACGAAGTTATGCTAGCGGATCCGAA |
| | TTCTTTTTTGCTCACCTGTGATTGC |
| | (SEQ ID NO: 33) |
| 2XgRNA-6R | ATTTAAATATTGCCCAATTCCCAATTCCCGGTACC |
| | AAAAAAAAAAGCACCGACTCGGTGC |
| | (SEQ ID NO: 34) |
| 1098A.C1F | AAGTGGCACCGAGTCGGTGCTTTTTTTTTTGCCTA |
| | CCTGGAGCCTGAGAGTTGTTCAATA |
| | (SEQ ID NO: 35) |
| 1098A.C2R | AATAACGGCCAGAGAAATTTCTCGACTCGAGACAA |
| | AAGCTGGAGCTCCTGCAGGTTGTTG |
| | (SEQ ID NO: 36) |
| 1098A.C3F | CAGGAGCTCCAGCTTTTGTCTCGAGTCGAGAAATT |
| | TCTCTGGCCGTTATTCGTTATTCTC |
| | (SEQ ID NO: 37) |
| 1098A.C6R | TGACCTACATCGTCGACACTAGTGGATCAGGCCTC |
| | CGGCCGTTAACTCGAATCGCTATCC |
| | (SEQ ID NO: 38) |
| Sxl-3BF | CACGTTATGCGTTCTCGCCACAGGATACAG |
| | (SEQ ID NO: 39) |
| Sxl-4AR | GCGAGTCCATTTCCGATGTGAAGTCCACG |
| | (SEQ ID NO: 40) |
| Tra-5AF | GGATGAGAATGAACGCTGGACGACCAGAC |
| | (SEQ ID NO: 41) |
| Tra-6AR | CTGTGATCTGGATCTGGAGCGAGTGCG |
| | (SEQ ID NO: 42) |
| βTub-1BF | GGCTTCCAGCTGACCCACTCGCTGGG |
| | (SEQ ID NO: 43) |
| βTub-2AR | CCAGATGGTTCAGGTCACCGTAGGTGG |
| | (SEQ ID NO: 44) |
| RPL32-F | CCAAGATCGTGAAGAAGCGC |
| | (SEQ ID NO: 45) |
| RPL32-R | CGCTTGTTCGATCCGTAACC |
| | (SEQ ID NO: 46) |
| ATPsynCF6-F | CCTGAACAAGGCCTCCGATC |
| | (SEQ ID NO: 47) |
| ATPsynCF6-R | CATCGCTGCCAAACTGCTTG |
| | (SEQ ID NO: 48) |
| Cas9-F | CCATATCGTGCCTCAGAGC |
| | (SEQ ID NO: 49) |
| Cas9-R | GTAATCAGCTTGGCGTTCAGC |
| | (SEQ ID NO: 50) |

The underlined sequence indicates the PAM.

Given that generation times in Drosophila melanogaster are faster at 26° C., the possibility of raising trans-heterozygous flies was tested at this temperature. Therefore, trans-heterozygous flies (dgRNA^{sxl,βTub}/+; Hsp70Bb-Cas9/+, dgRNA^{traA,βTub}/+; Hsp70Bb-Cas9/+ and dgRNA^{traB,βTub}/+; Hsp70Bb-Cas9/+) were raised at 26° C. and the sex ratios and fertility of emerging flies were scored. Unexpectedly, it was found that progeny from these flies could not be maintained at 26° C., since all F₁ females perished during development, or were converted into sterile intersexes ( [symbol] ), in 12.7±3.5% and 41.9±2.5% of cases, respectively (FIGS. 3C & 3E). However, the emerging trans-heterozygous males were fertile, indicating that male sterilization will require additional expression of the CRISPR components (FIGS. 3C-3E, Table 2). Taken together, these data suggest that the system is sufficiently leaky at 26° C. to kill female progeny, yet not leaky enough to sterilize male progeny.

TABLE 2
Assessing Temperature Inducible split-pgSIT systems: sex and fertility of F1
adult flies emerged under different temperature profiles.

| [symbol] | [symbol] | | | | | | | | | | [symbol] | | | | | | | | | [symbol] | | | | | | | | | |
|---|---|---|---|---|---|---|---|---|---|---|---|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | [symbol] | | | | | | | | | | [symbol] | | | | | | | | | [symbol] | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | [symbol] | | | | | | | | | | [symbol] | | | | | | | | | [symbol] | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |
| [symbol] | | | | | | | | | | | | | | | | | | | | | | | | | | | | | |

[symbol]

indicates data missing or illegible when filed

Temperature-Inducible Phenotypes

To identify the optimal heat-shock conditions required for the complete penetrance of desired TI-pgSIT phenotypes in F₁ progeny (i.e., female lethality and male sterility), Applicant heat shocked (37° C.) F₁ progeny raised at either 18° C. or 26° C. and quantified the sex ratios and fertility of emerging progeny. To determine the optimal heat-shock conditions, several conditions were compared. At 18° C., Applicant compared the development with no heat shock (18° C.^{NHS}); a 1-hr heat shock at the 1^{st} instar larval stage (18° C.^{1 HR-37° C.}); or a 4-hr heat shock at the 1^{st} instar larval stage (18° C.^{4 HR-37° C.}). At 26° C., Applicant compared the development with no heat shock (26° C.^{NHS}) or with a 2-hr heat shock at the 1^{st} instar larval stage (26° C.^{2 HR-37° C.}) (FIGS. 3C-3E, Table 2). The 18° C.^{1 HR-37° C.}) condition killed most of the females expressing sxl and transformed the surviving dgRNA^{sxl,βTub}/+; Hsp70Bb-Cas9/+ and dgRNA^{traA,βTub}/+; Hsp70Bb-Cas9/+ trans-heterozygous females into sterile intersexes (FIGS. 3C-3D, Table 2). However this condition was insufficient to transform/kill dgRNA^{traB,βTub}/+; Hsp70Bb-Cas9/+ trans-heterozygous females expressing U6.3-gRNA^{traB} (FIG. 3E). Interestingly, simply increasing the heat-shock period to 4 hours (18° C.^{4 HR-37° C.}) completely eliminated the gRNA^{sxl,βTub}/+; Hsp70Bb-Cas9/+ females (FIG. 3C) and transformed all gRNA^{traB,βTub}/+; Hsp70Bb-Cas9/+ females into intersexes (FIG. 3E, Table 2). Notwithstanding the complete transformation and killing of females observed above, none of the 18° C.^{4 HR-37° C.}. 18° C.^{1 HR-37° C.}, and 26° C.^{NHS} conditions ensured the complete sterility of F₁ trans-heterozygous males (FIGS. 3C-3E). Given these results, trans-heterozygous F₁ progeny was raised at 26° C. with a 2-hr heat shock at the 1^{st} instar larval stage (26° C.^{2 HR-37° C.}) which resulted in the development of sterile males and/or sterile intersexes for each trans-heterozygous combination (FIGS. 3C-3E, Table 2). Notably, gRNA^{traB,βTub}/+; Hsp70Bb-Cas9/+ intersex individuals were not identified under the 26° C.^{2 HR-37° C.}. Taken together, these results indicate that Hsp70Bb-Cas9 can direct the temperature-inducible expression of Cas9, which is sufficient to cause the 100% penetrance of the desired TI-pgSIT phenotypes. However careful titration is necessary to optimize the temperature conditions to achieve the desired phenotypes.

Simplified One-Locus TI-pgSIT

Given that both the designed trans-heterozygous combinations generated fertile flies when raised at 18° C. and only sterile males when heat shocked (26° C.^{2 HR-37° C.} FIGS. 3C-3E), TI-pgSIT systems that function in cis were tested to further simplify the approach. Therefore, two new constructs were engineered combining Hsp70Bb-Cas9 and one of two best dgRNA, gRNA^{sxl,βTub} and gRNA^{traB,βTub}, hereafter referred to as TI-pgSIT^{sxl,βTub,Hsp-Cas9} and TI-pgSIT^{traB,βTub,Hsp-Cas9}, respectively (FIG. 2C). Each TI-pgSIT cassette was site-specifically inserted into an attP docking site located on the 3rd chromosome (P{CaryP}attP2) using ϕC31-mediated integration (Groth et al., Genetics vol. 166 1775-1782 (2004)) to enable direct comparisons between the two systems. Both TI-pgSIT^{sxl,βTub,Hsp-Cas9} and TI-pgSIT^{traB,βTub,Hsp-Cas9} transgenic lines were generated and maintained as heterozygous balanced flies for >10 generations at 18° C. While a homozygous line was unable to be generated for TI-pgSIT^{sxl,βTub,Hsp-Cas9}, one was obtained for TI-pgSIT^{traB,βTub,Hsp-Cas9}.

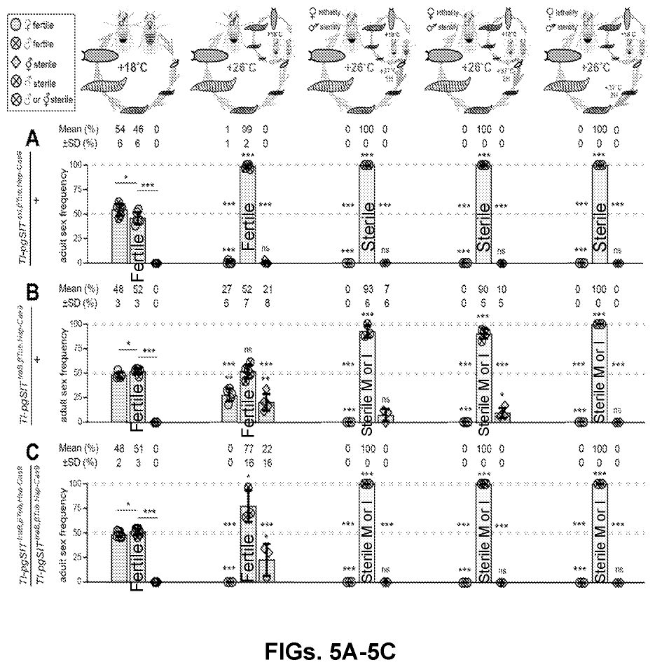

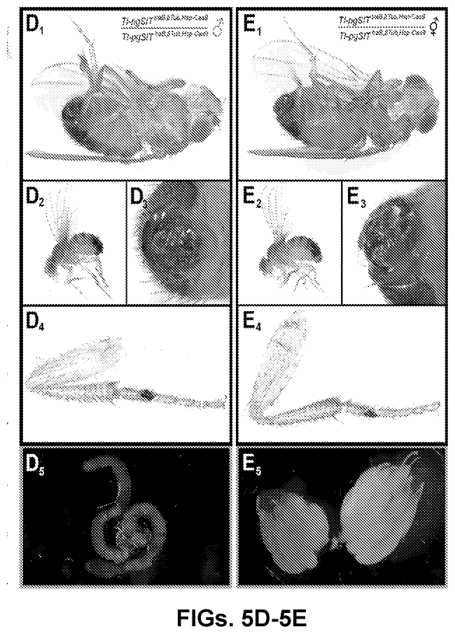

To assess the baseline expression of the one-locus TI-pgSIT systems at 18° C., the female-to-male ratio and fertility were evaluated in lines harboring a copy of either the TI-pgSIT^{sxl,βTub,Hsp-Cas9} or TI-pgSIT^{traB,βTub,Hsp-Cas9} cassette. A slightly female biased ratio for TI-pgSIT^{sxl,βTub,Hsp-Cas9}/+ line was found maintained at 18° C.: 54.5±6.0% ♀ vs 45.5±6.0% ♂ (p=0.025, a two-sided Student's t test with equal variance; FIG. 5A, Table 3). The TI-pgSIT^{traB,βTub,Hsp-Cas9} line had a slightly male biased ratio: 47.9±2.8% ♀ vs 52.0±8.3% ♂ for heterozygous flies (p<0.030, a two-sided Student's t test with equal variance; FIG. 5B, Table 3); and 48.4±2.4% ♀ vs 51.2±2.9% ♂ for homozygous flies (p<0.044, a two-sided Student's t test with equal variance; FIG. 5C, Table 3). Both heterozygous TI-pgSIT^{sxl,βTub,Hsp-Cas9} and homozygous TI-pgSIT^{traB,βTub,Hsp-Cas9} were maintained for nearly 2 years or more than 40 generations and counting at 18° C. Taken together, these experiments indicate that one-locus TI-pgSIT systems can be engineered, expanded, and maintained at 18° C. However, given that a homozygous line for TI-pgSIT^{sxl,βTub,Hsp-Cas9} could not be generated, careful titration of expression is necessary.

TABLE 3
Assessing one-locus TI-pgSIT systems: sex and fertility of adult flies
emerged under different temperature profiles.

| | [symbol] | | | | | | | | | | [symbol] | | | | | | | | | | [symbol] | | | | | | | | | |
|---|---|---|---|---|---|---|---|---|---|---|---|---|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |
| [symbol] | | | | | | | | | | | | | | | | | | | | | | | | | | | | | | |

[symbol]

indicates data missing or illegible when filed

Temperature-Inducible One-Locus TI-pgSIT

The effects of heat shock were tested on the penetrance of desired TI-pgSIT phenotypes. To activate the Hsp70Bb-Cas9 expression, eggs were collected from one-locus TI-pgSIT flies maintained at 18° C., and the staged eggs were raised at 26° C. with or without an additional heat shock at 37° C. Several different heat-shock conditions were compared including: the development from embryos to adult flies at 26° C. with no heat shock (26° C.^{NHS}); with a 1-hr heat shock at the 1st instar larval stage (26° C.^{1 HR-37° C.}) or with a 2-hr heat shock at the 1^{st} or 2^{nd} instar larval stages (26° C.^{2 HR-37° C.}). For the 26° C. condition, when TI-pgSIT^{sxl,βTub,Hsp-Cas9}/+ progeny were raised continuously at 26° C., this resulted in the near-complete elimination of females (54.5±6.0% ♀ at 18° C. vs 0.7±1.4% ♀ at 26° C., p<0.0001, a two-sided Student's t test with equal variance) with 98.5±2.0% of males emerging. However not all of these males were sterile (FIG. 5A, Table 3). Moreover, raising the flies with one or two copies of the TI-pgSIT^{traB,βTub,Hsp-Cas9} cassette at the 26° C.^{NHS} condition affected the sex ratio of the emerging progeny-some or all females, respectively, were transformed into intersexes, though the emerging males were still fertile (FIGS. 5B-5C, Table 3). Nevertheless, an additional 1-hr (26° C.^{1 HR-37° C.}) or 2-hr (26° C.^{2 HR-37° C.}) heat shock of the 1^{st} instar or the 2^{nd} instar larvae harboring either one copy of TI-pgSIT^{sxl,βTub,Hsp-Cas9} or two copies of TI-pgSIT^{traB,βTub,Hsp-Cas9} eliminated the females and intersexes and sterilized 100% of the males (FIGS. 5A-5C, Table 3). Taken together, these data indicate that heterozygous as well as homozygous viable strains harboring a one-locus TI-pgSIT genetic cassette can be generated and maintained at 18° C., and when progeny from these flies are simply grown at 26° C. and heat shocked during early larval development, the desired TI-pgSIT is fully penetrant.

Heat Shock Induces TI-pgSIT^{traB,βTub,Hsp-Cas9} Intersex Flies

It was previously observed that tra knockout (KO) induces an incomplete masculinization of D. melanogaster females converting them into intersexes (Kandul et al, 2019). To explore further what happens with TI-pgSIT^{traB,βTub,Hsp-Cas9} females under the 26° C.^{2 HR-37° C.} conditions, heat induced homozygous TI-pgSIT^{traB,βTub,Hsp-Cas9} males were thoroughly examined. It was noticed that several heat shocked TI-pgSIT flies developed extended abdomens. Dissections of their abdomens identified ovaries with oocytes (FIG. 5E₅). Therefore, without wishing to be bound by the theory, a fraction of TI-pgSIT^{traB,βTub,Hsp-Cas9} flies, which were raised under the 26° C.^{2 HR-37° C.} conditions, were indeed intersexes. These intersexes, unlike the heat shock induced dgRNA^{traA,βTub}/+; Hsp70Bb-Cas9/+ intersexes reared under the 26° C.^{2 HR-37° C.} (FIG. 3D) or the TI-pgSIT^{sxl,βTub,Hsp-Cas9} intersexes raised under the 26° C. without a heat shock (26° C.^{NHS} FIGS. 5B-5C), are difficult to distinguish from true males. The abdomen pigmentation (FIG. 5E₁₋₂), external genitals (FIG. 5E₃), and sex combs (FIG. 5E₃) of the TI-pgSIT^{traB,βTub,Hsp-Cas9} intersexes reared under 26° C.^{2 HR-37° C.} are nearly identical to those of males (FIG. 5D₁₋₄) prohibiting their correct identification (FIGS. 5B-5C). Therefore, to avoid intermixing true males with intersexes, the TI-pgSIT^{sxl,βTub,Hsp-Cas9} line was focused on for further experiments quantifying the basal Cas9 expression and assessing the competitiveness of heat-shocked sterile TI-pgSIT males.

Fitness and Basal Cas9 Expression

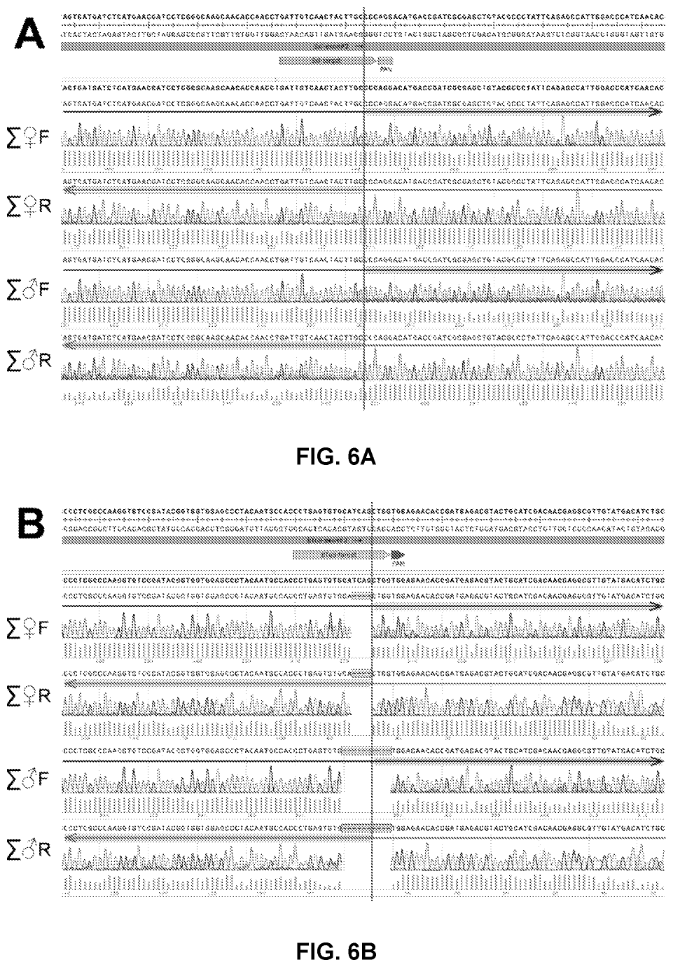

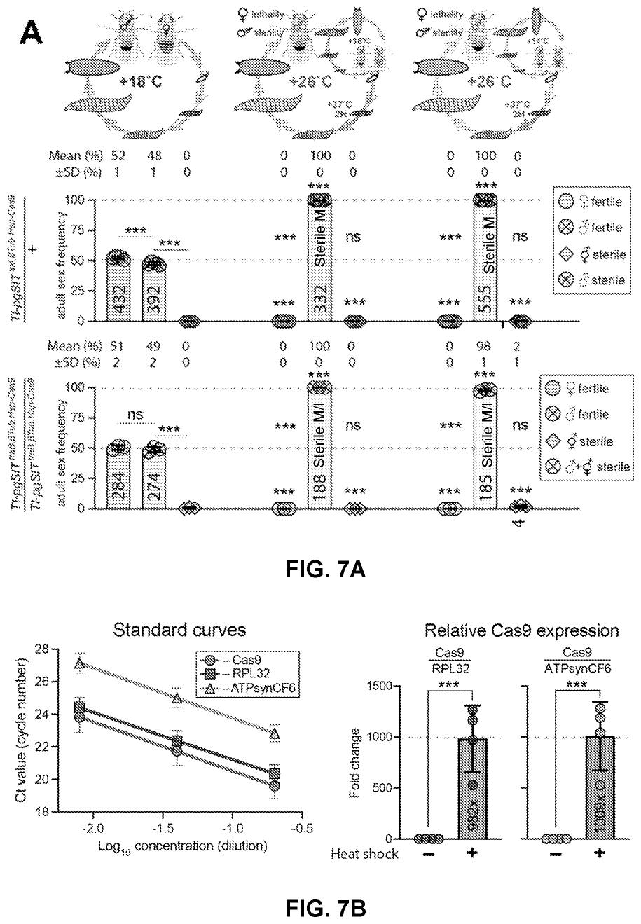

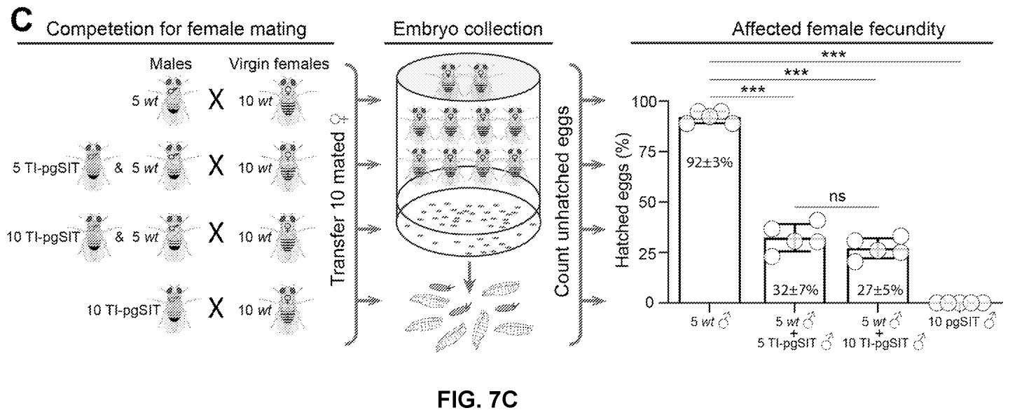

Attempts were made to establish the homozygous TI-pgSIT^{sxl,βTub,Hsp-Cas9} line, however homozygous females are only partially fertile and homozygous lineages cannot be maintained. To explore the reasons behind fitness costs of two copies of TI-pgSIT^{sxl,βTub,Hsp-Cas9} genetic cassette, both sxl and βTub target sequences were examined in flies raised at 18° C. Using Sanger sequencing, it was found that both target sequences were mutagenized resulting in ambiguous sequence reads downstream from the corresponding gRNA cut site (FIG. 6). These sequencing reads indicate that individual flies were likely mosaic for wt and indel (i.e. insertion and deletion) alleles at both sxl and βTub loci. However, it is not clear whether indel alleles were induced in only somatic cells or both somatic and germline cells. If functional indel alleles, which are resistant to Cas9/dgRNA^{sxl,βTub}-mediated cleavage, are induced in germline cells they will be selected and propagated through multiple generations. this possibility was examined by assessing the penetrance of heat induced pgSIT phenotypes (aka. female lethality/transformation and male sterility) using both TI-pgSIT^{sxl,βTub,Hsp-Cas9} and TI-pgSIT^{traB,βTub,Hsp-Cas9}/TI-pgSIT^{traB,βTub,Hsp-Cas9} lines after having maintained them for twelve months at 18° C. After heat-shocking multiple batches of larvae and analyzing large numbers of flies raised at 26° C., it was found that all females either perished or were transformed into intersexes while all resulting males were sterile (FIG. 7A). For TI-pgSIT^{sxl,βTub,Hsp-Cas9}/+, 877 sterile males and a single sterile intersex reared under the 26° C.^{2 HR-37° C.} condition were scored, vs 432 fertile females and 392 fertile males raised at 18° C. (FIG. 7A, Table 4). For TI-pgSIT^{traB,βTub,Hsp-Cas9}/TI-pgSIT^{traB,βTub,Hsp-Cas9}, scored were 377 sterile males and/or intersexes and no females (FIG. 5E₁₋₆) following the heat shock (26° C.^{2 HR-37° C.}) while 284 fertile females and 274 fertile males emerged under 18° C. (FIG. 7A, Table 4). Taken together, these results suggest that a basal Cas9 expression at 18° C. induces some indel alleles at sxl and βTub loci; however the leaky Cas9 expression is likely limited to somatic cells.

TABLE 4
Assessing one-locus TI-pgSIT systems after 12 months: sex and fertility of
adult flies emerged under different temperature profiles.

| | [symbol] | | | | | | | | | | | | [symbol] | | | | | | | | | | | | | |
|---|---|---|---|---|---|---|---|---|---|---|---|---|---|---|---|---|---|---|---|---|---|---|---|---|---|---|
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |
| [symbol] | | | | | | | | | | | | | | | | | | | | | | | | | | |

[symbol]

indicates data missing or illegible when filed

Quantification of Temperature-Induced Hsp70Bb-Cas9 Expression

To assess the extent of temperature induced Cas9 activation changes were quantified in the Cas9 mRNA relative to other genes using reverse transcription quantitative PCR (RT-qPCR). Two separate constitutively expressed genes, Ribosomal protein L32 (RPL32) and ATP synthase, coupling factor 6 (ATPsynCF6), were used for relative quantification of Cas9 expression. It was found that a 2-hr 37° C. heat shock and 4-hr 26° C. incubation of 2⁴ instar larvae induced a three-order-magnitude increase (1000 times) in the level of the Cas9 mRNA relative to that in the larvae maintained at 18° C. (FIG. 7B). Notably, two separate RT-qPCR qualifications based on independent normalizations genes (RPL32 and ATPsynCF6) inferred consistent estimations of the increase in Cas9 expression following the heat shock: 982X as Cas9/RPL32, and 1009X as Cas9/ATPsynCF6 (FIG. 7B). Notably, the larvae remaining were raised in the vials and it was verified that only sterile males emerged from vials raised under the 26° C.^{2 HR-37° C.} condition, while fertile females and males developed in vials maintained at 18° C. Therefore, a single copy of Hsp70Bb-Cas9 is sufficient to provide a three-order-magnitude transcription increase from its basal expression and induce efficient Cas9/gRNA-mediated mutagenesis, which in turn results in sxl and/βTub knockouts at the organismal level.

Competitiveness of Heat Induced TI-pgSIT^{sxl,βTub,Hsp-Cas9} Males

To explore potential fitness costs of activated Cas9 expression, the competitiveness of heat induced TI-pgSIT^{sxl,βTub,Hsp-Cas9} males were assessed. It was previously found that a single pgSIT male generated by crossing nanos-Cas9 and dgRNA^{sxl,βTub} were able to court and secure matings with ten wt females in the presence of one wt males (Kandul et al., 2019). To further increase competition between males, ten virgin females were confined with five TI-pgSIT or ten TI-pgSIT males in the presence of five wt males for 12 hours in the dark (FIG. 7C) before removing males and scoring egg hatching rate as female fecundity. Since heat induced TI-pgSIT males are sterile, eggs laid by the wt females that mated with TI-pgSIT males will not hatch, and significant decrease in female fecundity indicates that TI-pgSIT males are able to court, mate, and successfully compete with wt males. It was found that addition of five or ten TI-pgSIT sterile males to five fertile wt males resulted in a significant decrease in female fecundity, 91.9±3.0% vs 32.3±6.8 or 27.1±5.0%, respectively (P<0.0001, a two-sided Student's t test with equal variance, FIG. 7C). Interestingly, a single hatched egg was not scored out of 2179 eggs laid by females confined and mated with only TI-pgSIT males (FIG. 7C, Table 5) further supporting the induced male sterility of TI-pgSIT males. The mating competition assay indicated that the activated Hsp70Bb-Cas9 expression did not compromise the fitness of TI-pgSIT males and they were highly competitive with wt males at courting and mating with wt females.

TABLE 5
Assessing competitiveness of heat induced TI-pgSIT[dgRNA[Sxl,
βTub], Hsp-Cas9]/+ males.

| group | laid eggs | unhatched eggs | hatched egg % |
|---|---|---|---|
| 5 wt ♂ | 435 | 48 | 88.97 |
| 5 wt ♂ | 274 | 14 | 94.89 |
| 5 wt ♂ | 424 | 47 | 88.92 |
| 5 wt ♂ | 254 | 13 | 94.88 |
| 5 wt ♂ | 274 | 21 | 92.34 |
| 5 wt ♂ + 5 1098A ♂ | 119 | 83 | 30.25 |
| 5 wt ♂ + 5 1098A ♂ | 127 | 75 | 40.94 |
| 5 wt ♂ + 5 1098A ♂ | 111 | 77 | 30.63 |
| 5 wt ♂ + 5 1098A ♂ | 226 | 143 | 36.73 |
| 5 wt ♂ + 5 1098A ♂ | 234 | 180 | 23.08 |
| 5 wt ♂ + 10 1098A ♂ | 171 | 136 | 20.47 |
| 5 wt ♂ + 10 1098A ♂ | 287 | 192 | 33.10 |
| 5 wt ♂ + 10 1098A ♂ | 188 | 141 | 25.00 |
| 5 wt ♂ + 10 1098A ♂ | 215 | 159 | 26.05 |
| 5 wt ♂ + 10 1098A ♂ | 201 | 139 | 30.85 |
| 10 1098A ♂ | 240 | 240 | 0.00 |
| 10 1098A ♂ | 218 | 218 | 0.00 |
| 10 1098A ♂ | 205 | 205 | 0.00 |
| 10 1098A ♂ | 199 | 199 | 0.00 |
| 10 1098A ♂ | 250 | 250 | 0.00 |
| 20 1098A ♂ | 326 | 326 | 0.00 |
| 20 1098A ♂ | 475 | 475 | 0.00 |
| 20 1098A ♂ | 266 | 266 | 0.00 |

Provided herein is the proof-of-concept for a next-generation TI-pgSIT technology. TI-pgSIT addresses two major limitations of the previously described pgSIT (Kandul et al, 2019; Li et al; and Kandul et al., Reply to ‘Concerns about the feasibility of using “precision guided sterile males” to control insects’. Nature Communications vol. 10 (2019)). First, pgSIT relies on the separate inheritance of two required components, Cas9 endonuclease and gRNAs, that are activated in the F₁ progeny when combined by a genetic cross. As a result, two transgenic lines harboring either the Cas9 endonuclease or gRNAs must be maintained separately, which increases the production costs. Second, though the F₁ progeny of pgSIT undergo autonomous sex sorting and sterilization during development, enabling their release at any life stage, the genetic cross leading to the production of these F₁ sterile males requires the precise sex-sorting of parental Cas9 and gRNAs strains. Therefore, although pgSIT ensures the release of only sterile males, it still does not eliminate the insect sex-sorting step. Together these limitations can constrain applications of the original pgSIT technology for insect population control.

The TI-pgSIT system offers possible solutions to these limitations as it instead relies on a single pure-breeding strain, which eliminates the need for maintaining two strains that must still be sex sorted and mated in a facility for production of sterile males. One limitation of the TI-pgSIT approach is the heat-shock requirement during F₁ development, which would preclude the release of eggs. This means that the original pgSIT approach may be better suited for insects with a diapause during the egg stage (Kandul et al, 2019 and Li et al), though both the pgSIT and TI-pgSIT approaches will work well for the insects with a pupal diapause. Other than this limitation, the TI-pgSIT approach retains the benefits of the pgSIT technology, such as its non-invasiveness and high efficiency (Kandul et al, 2019). Also like the pgSIT approach, TI-pgSIT can in principle be engineered and applied to many insect species with an annotated genome and established transgenesis protocols. It utilizes CRISPR, which works in diverse species from bacteria to humans (Mojica & Montoliu. Trends Microbiol. 24, 811-820 (2016); Reardon. Nature 531, 160-163 (2016); and Reardon. Nature vol. 568 441-442 (2019)), to disrupt genes that are conserved across insect taxa, such as genes required for sex-determination and fertility. To establish TI-pgSIT in other species, a temperature-inducible promoter is needed. The heat-shock 70 proteins have high interspecies conservation in insects and play important roles in helping them survive under stressful conditions. The Drosophila Hsp70Bb promoter is one of the most studied animal promoters (Pelham and Bienz. 1982; and Dreano et al, 1986) and has been widely used for the heat-inducible expression of transgenes in many insect species for over 20 years (Morris et al., Nucleic Acids Res. 19, 5895-5900 (1991); Lycett & Crampton., Gene vol. 136 129-136 (1993); Matsubara et al., Proc. Natl. Acad. Sci. U.S.A. 93, 6181-6185 (1996); and Huynh & Zieler. J. Mol. Biol. 288, 13-20 (1999)). For example, Hsp70B promoters demonstrated robust heat-inducible expression of transgenes in the yellow fever mosquito, Aedes aegypti (Carpenetti, et al. Insect Mol. Biol. 21, 97-106 (2012)), the Mediterranean fruit fly, Ceratitis capitate (Kalosaka et al. Insect Mol. Biol. 15, 373-382 (2006)), and the spotted wing Drosophila, Drosophila suzukii (Ahmed et al., BMC Biotechnol. 19, 85 (2019)). This promoter should therefore be able to drive the heat-inducible expression of Cas9 in many insect species, especially when lower baseline expression is desirable (Kalosaka et al.). Moreover, the Hsp70Bb promoter could be ideal for inducing positively activated genetic circuits, as the activation of expression is rapid and does not require chemicals or drugs such as antibiotics, which can affect insect fitness directly (Ballard & Melvin; Zeh et al; and Chatzispyrou et al.) or indirectly by ablating their microbiomes (Wang et al; and Ourry et al.). Unlike common Tet-Off systems with conditional lethal transgenes (Thomas et al.; Fu et al., Nat. Biotechnol. 25, 353-357 (2007); and Schetelig et al., Insect Mol. Biol. 25, 500-508 (2016)) that are derepressed by withholding tetracycline, activation of the Hsp70Bb promoter is achieved by elevated temperatures. Heat-shock treatments can reduce maintenance costs compared to other inducible systems, as temperature is relatively costless compared to drugs and antibiotics.

Even though Cas9 expression was shown to be regulated by temperature using the Hsp70Bb promoter, the use of this promoter did result in some leaky expression. The leaky baseline expression of the Drosophila Hsp70Bb promoter in somatic cells at 25° C. is well known (Steller & Pirrotta; Bishop & Corces; and Bang & Posakony) and can be mitigated by either testing multiple genomic integration sites (Steller & Pirrotta) to titrate the leaky expression, or by targeting different genes. For example, two transgenic lines harboring each TI-pgSIT construct were generated. Flies harboring one or two copies of the TI-pgSIT^{traB,βTub,Hsp-Cas9} genetic cassette could be pure-bred and maintained at 18° C. However, only one copy of the TI-pgSIT^{sxl,βTub,Hsp-Cas9} genetic cassette could be maintained at this temperature, as its homozygous female were sterile at 18° C. Because these two lines are inserted at the same genomic insertion site, suggests that the target gene is important. Perhaps the regulation of sxl is more sensitive to mosaic mutations in somatic cells than that of tra, which would not be surprising as sxl is the master gene that controls both female development and X chromosome dosage compensation in D. melanogaster, and females homozygous for a loss-of-function mutation died due to the X chromosome hyperactivation (Cline. Genetics 90, 683-698 (1978)). Nevertheless, a multimerized copy of a Polycomb response element (PRE) could be used to attempt to further suppress the leaky Hsp70Bb-Cas9 expression (Akmammedov et al., Sci. Rep. 7, 6899 (2017).) and facilitate homozygousing an engineered TI-pgSIT cassette.

The Hsp70Bb-directed expression was reported to be suppressed in germline cells (Rørth. Mech. Dev. 78), 113-118 (1998)) even in response to heat-shock stimulation (Bonner et al., Cell 37, 979-991 (1984)). In Drosophila, the basic promoter of Hsp70Bb, which was incorporated in an upstream activation sequence (UASt) in the Ga14/UAS two-component activation system (Brand & Perrimon. Development 118, 401-415 (1993).), was shown to be targeted by Piwi-interacting RNAs (piRNAs) in female germline cells leading to degradation of any mRNA harboring endogenous Hsp70Bb gene sequences (DeLuca & Spradling. Genetics 209, 381-387 (2018)). Detected herein was the presence of indel alleles at both sxl and βTub target sites by Sanger sequencing these loci in TI-pgSIT^{sxl,βTub,Hsp-Cas9} flies raised at 18° C. After maintaining TI-pgSIT^{sxl,βTub,Hsp-Cas9}/+ and TI-pgSIT^{traB,βTub,Hsp-Cas9}TI-pgSIT^{traB,βTub,Hsp-Cas9} lines for twelve months at 18° C., the complete penetrance of heat induced pgSIT phenotypes (aka. female lethality/transformation and male sterility) was re-confirmed. Taken together, the results as shown herein are consistent with the absence of basal Cas9 expression in germline cells. The piRNA-mediated degradation of mRNA molecules harboring Hsp70Bb sequences in germline cells safeguards against generation and accumulation of mutant alleles. However, the three-order-magnitude activation (1000×) of Cas9 expression following the heat shock ensures the complete penetrance of both pgSIT phenotypes at the organismic level without compromising male mating competitiveness.

In summary, demonstrated herein is that by using a temperature-inducible CRISPR based approach, a single true-breeding strain can be maintained and the production of sterile and competitive males can be produced simply by shifting the temperature. This opens an entirely new approach for the generation of sterile males, and has now completely eliminated the need for sex sorting that is still required by other similar methods. TI-pgSIT could be adapted to both agricultural pests and human disease vectors to help increase the production of food and reduce human disease, respectively, thereby eliminating the need for harmful insecticides and revolutionizing insect population control.

### Experiment No. 2—Methods

### Assembly of Genetic Constructs

All genetic constructs generated in this disclosure were engineered using Gibson enzymatic assembly (Gibson et al. Nat. Methods 6, 343-345 (2009)). To assemble Hsp70Bb-Cas9^{dsRed} (FIG. 2A), the BicC-Cas9 plasmid (Kandul et al. Assessment of a Split Homing Based Gene Drive for Efficient Knockout of Multiple Genes. G3: GENES, GENOMES, GENETICS Feb. 1, 2020 vol. 10 no. 2 827-837) was digested with NotI and PmeI to remove the BicC promoter. The 476-base-long fragment encompassing the Hsp70Bb promoter and cloning overhangs were PCR amplified from the pCaSpeR-hs plasmid (GenBank #U59056.1) using primers 1137.C1F and 1137.C3R and cloned inside the linearized plasmid (Table 1). Then, the Hsp70Bb-Cas9-T2A-eGFP-p10 fragment was subcloned from Hsp70Bb-Cas9dsRed into the mini-white plasmid with the attB site. The dgRNA^{traB,βTub} plasmid was assembled following the strategy used to build dgRNA^{sxl,βTub} in a previous work (Kandul et al., 2019) (FIG. 2B). Briefly, the U6.3-gRNA^{traB} fragment was PCR amplified from the sgRNA^{traB} plasmid using primers 2XgRNA-5F and 2XgRNA-6R and was cloned into the sgRNA^{βTub} plasmid (Addgene #112691). To build the TI-pgSIT^{sxl,βTub,Hsp-Cas9} and TI-pgSIT^{traB,βTub,Hsp-Cas9} constructs (FIG. 2C), the U6.3 3′-UTR fragment was amplified using primers 1098A.C1F and 1098A.C2R from the pVG185_w2-yl plasmid (GenBank #MN551090.1) (Kandul et al., 2020) and the Hsp70Bb-Cas9-T2A-eGFP-10 fragment was amplified using primers 1098A.C3F and 1098A.C6R from the Hsp70Bb-Cas9 plasmid. Both were cloned into the dgRNA^{sxl,βTub} (Addgene #112692) or dgRNA^{traB,βTub} plasmid, respectively, after linearization at XbaI. The gRNA and primer sequences used to assemble the genetic constructs in the study are presented in Table 1.

Fly Transgenesis

Embryo injections were carried out at Rainbow Transgenic Flies, Inc. (www.rainbowgene.com). φC31-mediated integration (Groth et al) was used to insert the Hsp70Bb-Cas9^{dsRed} construct at the PBac{y+-attP-3B}KV00033 site on the 3^{rd} chromosome (BDSC #9750) and to insert the Hsp70Bb-Cas9^{dsRed} construct at the P{CaryP}attP2 site on the 3rd chromosome (BDSC #8622). The dgRNA^{traB,βTub} construct was inserted at the P{CaryP}attP1 site on the 2nd chromosome (BD SC #8621), and the TI-pgSIT^{sxl,βTub,Hsp-Cas9} and TI-pgSIT^{traB,βTub,Hsp-Cas9} constructs were inserted at the P{CaryP}attP2 site on the 3rd chromosome (BDSC #8622). The embryos injected with the TI-pgSIT^{sxl,βTub,Hsp-Cas9} and TI-pgSIT^{traB,βTub,Hsp-Cas9} constructs and any of their progeny starting from the G₁ generation were maintained at 18° C. Recovered transgenic lines were balanced on the 2^{nd} and 3^{rd} chromosomes using single-chromosome balancer lines (w¹¹¹⁸; CyO/sna^{Sco} for II and w¹¹¹⁸; TM3, Sb¹/TM6B, Tb¹ for III).

Fly Maintenance and Genetics

Flies were examined, scored, and imaged on the Leica M165FC fluorescent stereo microscope equipped with the Leica DMC2900 camera. The inheritance of Hsp70Bb-Cas9^{dsRed} was tracked using the Opie2-dsRed genetic marker. The other transgenes were tracked using the mini-white marker. All genetic crosses were performed in the w-genetic background. Flies harboring both Hsp70Bb-Cas9 and dgRNAs in the same genetic background were maintained at 18° C. with a 12H/12H light and dark cycle, while the flies harboring either Hsp70Bb-Cas9 or dgRNAs were raised under standard conditions at 26° C. All genetic crosses were performed in fly vials using groups of seven to ten flies of each sex.

The heat-shock-induced activation of Hsp70Bb-Cas9 was assessed by visualizing GFP fluorescence. The GFP coding sequence was attached to the C-terminal end of the Streptococcus pyogenes-derived Cas9 (SpCas9) coding sequence via a self-cleaving T2A peptide and served as a visual indicator of Cas9 expression. The embryos that were laid overnight as well as the larvae, pupae, and adult flies of both Hsp70Bb-Cas9 and TI-pgSIT^{traB,βTub,Hsp-Cas9} homozygous lines were heat shocked for two hours at 37° C., and in 6, 15, or 24 hours post heat shock, their GFP expression was imaged and compared to that of the non-treated embryos, larvae, pupae, or flies raised at 18° C. or 26° C. To assess the inducible expression of Hsp70Bb-Cas9 directly, the Cas9/dgRNA knockout phenotypes induced by a heat shock were compared to those without the heat shock. Two different double guide RNA (dgRNA) (dgRNA^{sxl,βTub} and dgRNA^{traB,βTub}) lines with the same Hsp70Bb-Cas9 line were tested as the F¹ trans-heterozygotes the classic pgSIT. The homozygous dgRNA and Cas9 lines were genetically crossed, and their trans-heterozygous embryos were raised at either 18° C. or 26° C. Additionally, groups of these embryos underwent various durations of heat shocks at 37° C. during the 1^{st} or 2^{nd} day post oviposition (FIG. 3). For heat-shock treatments, glass vials with staged embryos and/or larvae were incubated in a water bath at 37° C. Different temperature conditions were tested to assess the induction levels between the baseline and complete expression of Cas9 for each dgRNA construct: the development at 18° C. with no heat shock (18° C.^{NHS}), a 1-hr heat shock at the 1^{st} instar larval stage (18° C.^{1 HR-37° C.}) or a 4-hr heat shock at the 1^{st} instar larval stage (18° C.^{4 HR-37° C.}) The development at 26° C. was tested with no heat shock (26° C.^{NHS}) or with a 2-hr heat shock at the 1^{st} instar larval stage (26° C.^{2 HR-37° C.}) (FIG. 3).

The generated transgenic lines harboring one or two copies of TI-pgSIT^{sxl,βTub,Hsp-Cas9} and TI-pgSIT^{traB,βTub,Hsp-Cas9} genetic cassettes were maintained for >10 generations at 18° C. To induce the pgSIT phenotypes, staged embryos were generated at 18° C. and shifted to 26° C. to complete their development. Different temperature conditions were tested to fully activate the Cas9 expression: the development at 18° C. with no heat shock (18° C.^{NHS}) and the development at 26° C. with no heat shock (26° C.^{NHS}) a 1-hr heat shock at the 1^{st} instar larval stage (26° C.^{HR-37° C.}), or a 2-hr heat shock at the 1^{st} or 2^{nd} larval stages (26° C.^{2 HR-37° C.}) (FIG. 5). To estimate the efficiency of knockout phenotypes, the sex of emerging adult flies was scored as female (♀), male (♂), or intersex ( [symbol] ) and tested the fertility of generated flies as previously described in Kandul et al. (Kandul et al., 2019). Note that the induced male sterility was tested in multiple groups of 7-20 males per group from the same biological sample. A single fertile male would designate an entire sample as fertile. Each experimental test was repeated a minimum of three times for statistical comparisons.

Genotyping Loci Targeted with gRNAs

The molecular changes that caused female lethality and male sterility were examined following the previously described protocol (Kandul et al., 2019). Briefly, the sxl, tra, and βTub loci targeted by the gRNAs were PCR amplified from individual flies and were sequenced in both directions using the Sanger method at GENEWIZ®. The sequence reads were aligned against the corresponding reference sequences in SNAPGENE® 4. The primer sequences used for the PCR of the sxl, tra, βTub loci are presented in Table 1. Also sequenced were sxl and βTub loci using DNA extracted from multiple TI-pgSIT^{sxl,βTub,Hsp-Cas9} females or males reared at 18° C. to assess leaky Hsp70Bb-Cas9 expression in somatic cells.

Reverse Transcription Quantitative PCR (RT-qPCR)

The TI-pgSIT^{sxl,βTub,Hsp-Cas9} line was used to quantify the activation Hsp70Bb-Cas9 expression. Vials containing staged larvae were maintained at 18° C. Heat treated vials were incubated in the heat block for 2 hours at 37° C. and then for 4 hours at 26° C. The not-heat-treated vials stayed at 18° C. Larvae were separated from food in room-temperature water. Total RNA was extracted using the RNeasy Mini Kit (Qiagen), quantified using the NanoDrop 2000 (THERMO SCIENTIFIC™), and then treated with DNAse I (THERMO SCIENTIFIC™) following the protocol. cDNA was synthesized using the RevertAid First Strand cDNA Synthesis Kit (THERMO SCIENTIFIC™) with a primer mixture of 1:6 of Oligo (dT) is primer and random hexamer primers. Real-time qPCR was performed using LIGHTCYCLER® 96 Instrument (Roche). RT-qPCR quantification of Hsp70Bb-Cas9 expression was done relative to RPL32 and ATPsynCF6. Reversed transcribed cDNA samples from not-heat-treated replicates were serially diluted over 50× to build standard curves for each amplified gene fragment and test primer performance (Table 1). A 10× dilution of cDNA (middle of the standard curve range) was used for relative quantification of Hsp70Bb-Cas9 expression. Real-time quantification PCR reactions (20 μL) contained 4 μl of sample, 10 μl of SYBR Green Master Mix, 0.8 μl of forward primer and 0.8 μl of reverse primer and 4.4 μl of ultrapure water. Negative control (20 μl) contains 10 μl of SYBR Green Master Mix, 0.8 μl of forward primer and 0.8 μl of reverse primer and 8.4 μl of ultrapure water. Three technical replicates were run per place for each of four biological replicates. The Real-time quantification PCR data were analyzed in LIGHTCYCLER® 96 Application (Roche Applied Science) and exported into an Excel datasheet for further analysis. RNA levels were normalized to RPL32 or ATPsynCF6 to generate two separate relative quantifications of Hsp70Bb-Cas9 mRNA after a two-hour heat shock.

Competition Assay of TI-pgSIT Males

The competitiveness of the induced TI-pgSIT^{sxl,βTub,Hsp-Cas9} males was evaluated by their ability to mate with females in the presence of wt males. It was previously demonstrated that one fertile male is able to mate with the majority of ten virgin females in twelve hours (Kandul et al., 2019). To increase mating competition, 10 virgin females were confined with 5 wt males alone, 5 wt and 5 TI-pgSIT males, 5 wt and 10 TI-pgSIT males, or 10 TI-pgSIT males alone in a vial for 12 hours in the dark. As previously, freshly emerged induced TI-pgSIT and wt males were isolated from females and aged for 4 days before the competition assay to increase the male courtship drive. After 12 hours of mating, the females were transferred into small embryo collection cages (Genesee Scientific 59-100) with grape juice agar plates and percentage of hatched eggs were calculated. The decrease in female fecundity, estimated by a number of unhatched eggs, indicated the ability of a sterile TI-pgSIT male to score successful matings with females in the presence of a wt male; and thus provided a readout of the competitiveness of the induced TI-pgSIT^{sxl,βTub,Hsp-Cas9} males.

Statistical analysis. Statistical analyses were performed in JMP 8.0.2 by SAS Institute Inc. Three to five biological replicates were used to generate statistical means for comparisons. P values were calculated for a two-sided Student's t test with equal variance.

Data availability. All data underlying FIGS. 3&5 are represented fully within Tables 2-4. The plasmids constructed in the study were deposited at Addgene.org (#149424-149427, FIG. 2). The Hsp70Bb-Cas9, Hsp70Bb-Cas9^{dsRed} dgRNA^{traB,βTub}, TI-pgSIT^{sxl,βTub,Hsp-Cas9} and TI-pgSIT^{traB,βTub,Hsp-Cas9} transgenic lines were deposited to the Bloomington Drosophila Stock Center.

### Experiment No. 3—One-Locus Inducible Precision Guided Sterile Insect Technique

Large sterile male releases are the gold standard for most insect population control methods and precise sex sorting is essential to the success of these technologies. Efficient removal of females prior to releasing sterile males is especially important for mosquito control because female mosquitoes bite and transmit diseases. It was previously developed the CRISPR-mediated precision guided Sterile Insect Technique (pgSIT) method to program the female-specific lethality and male-specific sterility into the development of F₁ eggs without incurring fitness cost to the released males. However, the current pgSIT method requires breeding two (Cas9 and gRNA) strains and sex-sorting of parent insects for the genetic cross to generate large numbers of F₁ eggs in a factory. Described herein is a one-locus pgSIT technology that relies on the inducible expression of Cas9 in a single pure-bred pgSIT line. To achieve the positive activation of Cas9 expression, the promoter of heat-shock protein 70Bb (Hsp70Bb) was tested. Two separate one-locus pgSIT transgenic lines were engineered and their proof-of-concept performance was demonstrated in Drosophila melanogaster. Though both lines have been pure-bred in the laboratory for more than 10 generations at +18° C., heat-shocking their eggs for 1 hour at +37° C. followed by development at +26° C. has consistently resulted in 100% female lethality and male sterility. Since, this system does not require application of any drugs and/or antibiotics, and after a brief heat-shock, insects are maintained under a normal temperature, the pgSIT induction does not affect the fitness of the emerging sterile males. By accomplishing three tasks simultaneously—sex-sorting, female removal, and male sterilization—the one-locus pgSIT technology truly automates the production of large numbers of sterile insect males. Therefore, this technology revolutionizes insect population control.

The ability to generate and release large numbers of males that are not able to sire progeny into natural populations is a key requirement for many current approaches in insect population control. In 1955, Edward Knipling proposed releasing sterile males to suppress insect populations in so-called Sterile Insect Technique (SIT). Since then, SIT was successfully implemented to eradicate Cochliomyia hominivorax, the new world screw-worm fly, in the U.S. and Mexico and suppress populations in a variety of other insects. However, Knipling's goal of sexing sterilized insects by removing females prior to their release was challenging to accomplish even in C. hominivorax and has limited the implementation of SIT to agricultural pests. Field trials and models clearly showed that releasing only sterile males, which were not distracted by accompanying females, significantly improves the efficiency of population suppression and can result in significant cost reduction. Other related methods of insect control, such as Release of Insects carrying a Dominant Lethal (RIDL) and the Wolbachia-mediated Incompatible Insect Technique (IIT), also require consistent sexing methods to avoid female releases. Notably, IIT programs are based on the release of Wolbachia infected males incompatible with the wild type females, which are free of a Wolbachia strain, and the accidental release of only a small proportion of Wolbachia infected fertile females can immunize a target population against the Wolbachia strain used in a particular IIT program. Furthermore, since mosquito females are blood feeders and transmit many diseases, a reliable sexing method to guarantee female elimination among released insects is highly desirable for the implementation of SIT, RIDL, or IIT programs in mosquito species.

Recently developed was the CRISPR-mediated precision guided SIT (pgSIT) technology that addresses the major shortcomings of current methods for insect population control (Kandul et al., 2019). The pgSIT relies on the Cas9/gRNA cleavage to knock out the female-specific viability and male-specific fertility genes simultaneously and generate 100% male progeny without incurring fitness costs. Two homozygous strains harboring Cas9 and gRNA transgenes can be maintained separately in an insect factory, while their genetic cross produces the F₁ eggs that autonomously develop into 100% sterile males; and can be released as eggs for insect population suppression, facilitating the deployment logistics for the insects species with diapausing eggs, such as Aedes aegypti and Aedes albopictus mosquitos (Hanson et al., J. Am. Mosq. Control Assoc. 9, 78-83 (1993); Rezende et al. BMC Developmental Biology vol. 8 82 (2008); and Brown et al. Journal of Medical Entomology tjw186 (2016) doi: 10.1093/jme/tjw186). Furthermore, models using the available field parameters found that pgSIT outperformed current methods of population suppression, such as RIDL, Wolbachia IIT, and female specific RIDL. However, while the current pgSIT accomplishes the 100% efficient sex-soring of the F1 eggs by inducing lethality of all female progeny, it still requires the sex-sorting at an insect factory to collect virgin females and males from two homozygous Cas9 and gRNA strains for the genetic cross generating F₁ eggs. To amend this limitation of the pgSIT, the one-locus inducible pgSIT was engineered as described herein.

Described herein is a one-locus inducible pgSIT design and its proof-of-concept was demonstrated in Drosophila melanogaster. The Heat-shock protein 70Bb (Hsp70Bb) promoter was used to engineer the inducible expression of SpCas9 protein and, in conjunction with the constitutive expression of two gRNAs, resulted in the pgSIT phenotypes. Two one-locus pgSIT genetic cassettes were generated and established as Drosophila pure-bred strains: (1) pgSIT Sxl,βTub,Hsp70Bb-Cas9 and (2) pgSIT TraB,βTub,Hsp70Bb-Cas9. It was found that the transgenic line harboring one or two copies of the one-locus pgSIT genetic cassette could be maintained for more than 10 generations at +18° C., while one or two hours of the +37° C. heat shock during early development with the subsequent maintenance at +26° C. induced the Hsp70Bb-Cas9 expression sufficient to cause 100% female lethality and 100% male sterility. The one-locus pgSIT design represents a qualitative improvement in the current split-pgSIT technology. It significantly cuts the costs associated with the maintenance of transgenic lines and completely automates sex-sorting. Accordingly, the one-locus pgSIT technology qualitatively advances the insect population control.

Temperature inducible activation by the heat-shock protein 70B b (hsp70B b) promoter. To generate an inducible approach that does not require exposure to chemicals/antibiotics, known to impact the fitness of released animals, developed herein was a temperature inducible activation system. The classic Hsp70Bb (Hsp70, CG31359) promoter was utilized to establish a temperature inducible activation system for Cas9 expression. The expression of Hsp70Bb is known to be activated by raising temperature to 37° C., a heat-shock, and rapidly decrease to preshock levels following a return to the normal temperature of 25° C. The Hsp70Bb-Cas9-T2A-eGFP-p10 (Hsp70Bb-Cas9) construct was engineered (FIG. 8A) and established a homozygous strain of Drosophila melanogaster. Downstream (3′) to the Hsp70Bb-driven Cas9, a self-cleaving T2A peptide and eGFP coding sequence were included, together serving as a visual indicator of promoter activity. To assess the activity of Hsp70Bb-Cas9, the GFP fluorescence in the 2nd instar Hsp70Bb-Cas9 larvae that were raised under 18° C. was compared to those raised under 26° C. plus a one-hour heat-shock at 37° C., and it was found that the heat-shocked larvae had brighter GFP fluorescence than those maintained under 18° C.

The low expression level of Hsp70Bb-Cas9 under the permissive temperature. To maintain and pure-breed the one-locus pgSIT insects, none or a very low expression of Cas9 endonuclease, in conjunction with the constitutive expression of dgRNAs, can be tolerated under the permissive temperature. To eliminate the female progeny or transform them into phenotypic males under a restrictive temperature, gRNA targeting genes at the top of the sex-termination pathway, sex lethal (Sxl) and transformer (tra), were investigated. Double sex (dsx) was excluded, whose knockout resulted in the less-pronounced intersex phenotype. Three dgRNA sxl,βTub, dgRNA tra,βTub, dgRNA traB,βTub lines, each constitutively expressing two gRNAs targeting βTub and sxl or tra (FIG. 8B), were evaluated together with Hsp70Bb-Cas9 to assess the extent of the basic Cas9 expression under the permissive temperature, 20° C. Homozygous dgRNA and Hsp70Bb-Cas9 virgin flies of the opposite sex were allowed to mate and lay eggs overnight at 26° C. before moving parent flies into new vials, and raising staged F₁ trans-heterozygous embryos at 20° C.

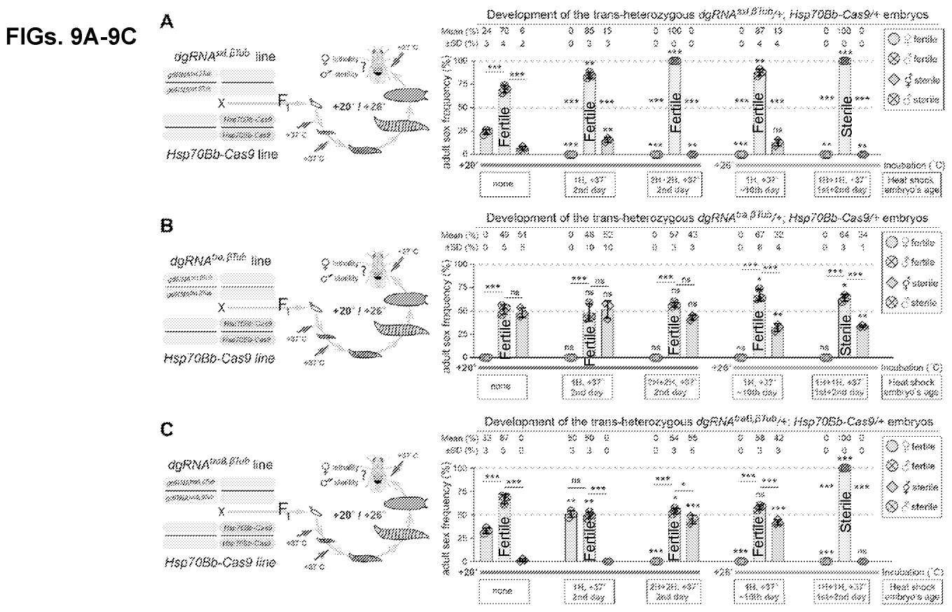

Two out of three types of F₁ trans-heterozygous embryos developed into fertile adult females and males under 20° C., though significantly fewer females than males emerged. The dgRNA sxl,βTub/+; Hsp70Bb-Cas9/+ embryos developed as 23.7±2.7% of females and 69.8±3.8% of males (♀ vs ♂, P<0.0001, a two-sample Student's t-test with equal variance; FIG. 9A); and the development of dgRNA traB,βTub/+; Hsp70Bb-Cas9+ resulted in 33.0±3.3% of females and 66.9±5.0% of males (♀ vs ♂, P<0.0006, a two-sample Student's t-test with equal variance; FIG. 9C). To assess the fertility of both sexes emerged from the F₁ embryos generated by reciprocal crosses of dgRNA sxl,βTub or dgRNA traB,βTub and Hsp70Bb-Cas9, mating among emerged F₁ flies was permitted and a viable F 2 progeny was generated. Note that the original dgRNA sxl,βTub line together with Hsp70Bb-Cas9 induced the masculinization of all F₁ females into sterile intersexes when the trans-heterozygous embryos were raised at 20° C. resulting in similar ratios of sterile intersexes and fertile males in the F₁ progeny (48.5±5.1% [symbol] vs 51.5±5.1% ♂, P=0.507, a two-sample Student's t-test with equal variance; FIG. 9B). These results indicate that the Hsp70Bb promoter directed very limited expression of Cas9 under the permissive temperature enabling breeding of trans-heterozygous flies for at least one additional generation.

A heat-shock induces Hsp70Bb-Cas9 expression resulting in robust pgSIT phenotypes: female-specific lethality, and male-species sterility. To identify the minimal heat-shock duration required for the complete penetrance of pgSIT phenotypes in the Fi progeny, the duration of heat-shock was titrated under two different temperature profiles, ratios of emerging sexes were quantified, and their fertility was scored. First, a one- or four-hour heat-shock by 37° C. was added during an early developmental stage (two days after egg laying, during the 1st instar larvae stage) to the F₁ embryos raised at 20° C. A one-hour heat-shock was sufficient to ‘kill’ the majority of trans-heterozygous females harboring gRNA sxl, while 15.4±2.7% of females were masculinized into intersexes (FIG. 9A); and had no effect on the F₁ progeny harboring gRNA tra (FIG. 9B). Surprisingly, this heat-shock induced an equal sex ratio between fertile females and males emerging from the F₁ trans-heterozygous embryos carrying gRNA traB (50.5±3.4% ♀ vs 49.5±3.4% ♂, P=0.748, a two-sample Student's t test with equal variance, FIG. 9C). The four-hour heat-shock caused the complete elimination of the F₁ females with gRNA sxl, masculinized all the F₁ females with gRNA traB, and had no statistically significant effect on the F₁ progeny with gRNA tra (FIGS. 9A-9C). When the F₁ embryos were raised at 20° C., both short and long heat-shock did not cause sterility in emerging trans-heterozygous males. Therefore, raising trans-heterozygous progeny at 26° C. was tested with an additional heat-shock at the adult or early developmental stage. The elevated continuous temperature induced complete masculinization of all F₁ trans-heterozygous females, though the emerging F₁ males were not completely sterile, and an additional heat-shock of adult flies did not cause the male sterility (FIGS. 9A-9C).

Nevertheless, a combined two-hour heat-shock during the first days after egg laying together with the development at 26° C. caused complete sterility in emerging F₁ males for each trans-heterozygous combination. Notably, this temperature treatment of dgRNA sxl,βTub/+; Hsp70Bb-Cas9+ and dgRNA traB,βTub/+; Hsp70Bb-Cas9+ embryos generated 100% male-progeny, since all females and intersexes perished (FIGS. 9A & 9C). Taken together, these results indicate that Hsp70Bb-Cas9 can direct the temperature-inducible expression of Cas9 endonuclease that once activated is sufficient to cause the 100% penetrance of pgSIT phenotypes.

Two one-locus inducible pgSIT transgenic lines were established. The trans-heterozygous combinations of dgRNA sxl,βTub or dgRNA traB,βTub and Hsp70Bb-Cas9 were found to generate either fertile flies of both sexes under the permissive temperature or only sterile males under the restrictive temperature (FIGS. 9A & 9C). Therefore, two one-locus pgSIT genetic cassettes were built using these tested components, hereafter referred to as pgSIT sxl,βTub,Hsp70Bb-Cas9 and pgSIT traB,βTub,Hsp70Bb-Cas9, respectively (FIG. 8C). Each one-locus pgSIT cassette was injected at two attP sites located on the 2nd and 3rd chromosomes, P{CaryP}attP1 and P{CaryP}attP2, respectively, using the φC31-mediated integration. Although an extra care was taken to maintain the injected embryos and perform genetic screens at 18-20° C., it was failed to establish transgenic lines at the P{CaryP}attP1 site on the 2nd chromosome. Note that a few transgenic males and intersexes harboring the mini-white marker were identified for each one-locus pgSIT cassette suggesting a correct insertion at P{CaryP}attP1, though it was failed to establish and maintain balanced transgenic lines. Nevertheless, both pgSIT sxl,βTub,Hsp70Bb-Cas9 and pgSIT traB,βTub,Hsp70Bb-Cas9 transgenic lines were generated using the P{CaryP}attP1 site, and maintained as heterozygous balanced flies for more than 10 generations at 18° C. Furthermore, the homozygous line harboring two copies of the pgSIT traB,βTub,Hsp70Bb-Cas9 was pure-bred for more than 6 generations.

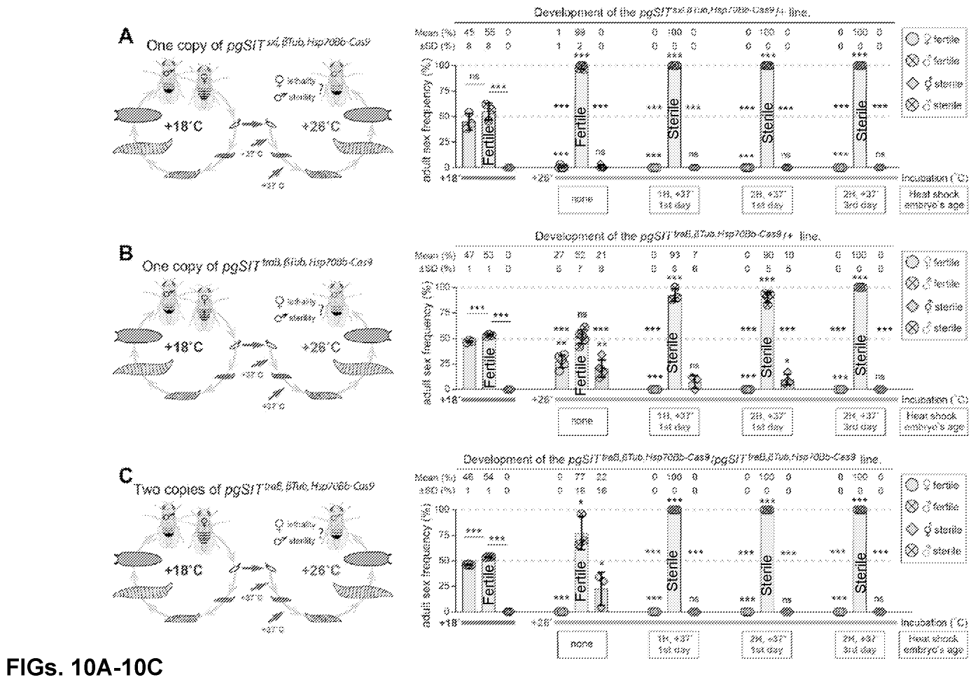

To assess the level of the basic expression of Hsp70Bb-Cas9 under the permissive temperature, the sex ratio was quantified and the fertility of both one-locus pgSIT transgenic flies harboring one copy of pgSIT sxl,βTub,Hsp70Bb-Cas9 or pgSIT traB,βTub,Hsp70Bb-Cas9 genetic cassette were examined. It was found that 45.3±8.3% of females and 54.7±8.3% of males emerged from the pgSIT sxl,βTub,Hsp70Bb-Cas9/+ line when it was maintained under 18° C. (♀ vs ♂, P=0.233, a two-sample Student's t test with equal variance; FIG. 10A). The pgSIT sxl,βTub,Hsp70Bb-Cas9/+ flies maintained under 18° C. generated the female-to-male progeny ratio that was similar to 50/50, and yet the female abundance was significantly smaller than that of males (47.0±0.9% ♀ vs 53.0±0.9% ♂, P<0.001, a two-sample Student's t test with equal variance; FIG. 10B). The similar male-biased ratio was found in the emerging progeny from the flies harboring two copies of the pgSIT sxl,βTub,Hsp70Bb-Cas9 genetic cassette (46.3±0.5% ♀ vs 53.7±0.5% ♂, P<0.0001, a two-sample Student's t test with equal variance; FIG. 10C). Multiple groups of progeny flies raised under the permissive temperature were also tested and it was found that they were mutually fertile, and generated the next generation of healthy progeny. To explore potential genetic changes at targeted loci induced by Cas9/gRNA at the permissive temperature, the target loci from single flies was sequenced. It was found that many tested flies had a few different alleles mapped at the gRNA target sequences. The analyzed sequence output indicated that a few wt alleles were mutated in some cells of the fly body. Nevertheless, these mutations did not accumulate over many generations and affect the pure-breeding of the one-locus pgSIT flies. The transgenic lines harboring one copy of each one-locus pgSIT cassette have been maintained for more than 10 generations while the homozygous one-locus pgSIT line has been pure-bred for 6 generations in the lab. These results indicate that an inducible one-locus pgSIT cassette remains largely inactive under the permissive temperature permitting pure-breeding of one-locus pgSIT lines.

The temperature-activated one-locus pgSIT genetic cassette caused 100% penetrance of pgSIT phenotypes. The effects of a heat-shock length and timing on the penetrance of induced pgSIT phenotypes were assessed: such as female-species lethality, and male-species sterility. To activate the Hsp70Bb-Cas9 expression, the one-locus pgSIT flies were shifted from 18° C. to the restrictive temperature of 26° C. First, the flies were maintained continuously at 26° C. and the progeny emerging from staged eggs was scored and analyzed. The continuous exposure of the pgSIT sxl,βTub,Hsp70Bb-Cas9/+ flies to the restrictive temperature resulted in a nearly complete lethality of female progeny (45.3±8.3% ♀ at 18° C. vs 0.7±1.4 ♀ at 26° C., P<0.0022, a two-sample Student's t test with equal variance) and 98.5±2.0% of males emerged, and yet at least some males were fertile (FIG. 10A). Raising the flies with one or two copies of the pgSIT traB,βTub,Hsp70Bb-Cas9 cassette at 26° C. affected the sex ratio of the emerging progeny, some or all females, respectively, were transformed into intersexes and emerging males were fertile (FIGS. 10B-10C). Nevertheless, an additional one-hour heat-shock at 37° C. of one-day-old larvae harboring one copy of pgSIT sxl,βTub,Hsp70Bb-Cas9 or pgSIT traB,βTub,Hsp70Bb-Cas9 resulted in the emergence of 100% sterile males. It was found that the heat-shock at 37° C. for at least one hour during early days of the development under the continuous 26° C. caused the complete penetrance of female-specific lethality and male-specific sterility in the transgenic flies carrying at least one copy of one-locus pgSIT genetic cassette (FIGS. 10A-10C). Taken together, these results indicate that one-locus pgSIT genetic cassettes engineered in this disclosure can be integrated into the Drosophila genomes, inherited silently over multiple generations under the permissive temperature, and temperature-activated when sterile males are required.

Described herein is the one-locus precision guided Sterile Insect Technique (pgSIT) and its proof-of-concept was demonstrated in Drosophila melanogaster. Its design is based on combining two genetic components, Cas9 endonuclease and gRNAs targeting genes required for female-specific viability and male-specific sterility, into a single, hence one-locus, genetic cassette. Since, the Drosophila U-6.3 promoter drives the constitutive expression of gRNAs, and the activity of an entire one-locus pgSIT genetic cassette is controlled by the Drosophila Hsp70Bb promoter directing Cas9 expression. It was show herein that the expression Cas9 endonuclease can be precisely regulated by an ambient temperature and additional heat-shocks. The tight control of an inducible Cas9 expression permits pure-breeding of the transgenic flies harboring a one-locus pgSIT genetic cassette; but, the shift to an elevated temperature plus a heat-shock activates the one-locus pgSIT cassette resulting in the production of 100% of sterile males for insect population control (FIG. 10).

A few genomic integration sites have to be assessed for each one-locus pgSIT genetic cassette to account for the effect of a genomic environment (Weiler & Wakimoto. Annu. Rev. Genet. 29, 577-605 (1995)) on the expression level of Cas9 and the general fitness of transgenic flies. It was found that transgenic flies harboring each one-locus pgSIT cassette at both P{CaryP}attP1 and P{CaryP}attP2 sites, but it was failed to establish pure-breeding lines for the P{CaryP}attP1 site under the permissive temperature. These results suggest that the P{CaryP}attP1 site supports the stronger basic expression of the one-locus pgSIT cassettes than that by the P{CaryP}attP1 site preventing the perpetual breeding the genetic cassettes at P{CaryP}attP1. Both one-locus pgSIT genetic cassettes integrated at the P{CaryP}attP1 site on the 3rd chromosome also support some level of basic expression of Cas9, thought this leaky expression must be quite low since it does not limit fertility of the one-locus pgSIT transgenic flies. Furthermore, the leaky expression of Cas9 is likely limited to somatic tissues and thus does not induce inheritable mutations. Even the flies harboring two copies of the one-locus pgSIT cassette have been continuously pure-bred for six generations so far.

The Hsp70Bb promoter may be an ideal inducible promoter to engineer positively activated genetic circuits for transgenic insects. The activation of heat-shock protein expression is rapid and does not require any chemicals or drugs such as antibiotics, which can affect insect fitness directly or indirectly by ablating their microbiomes. Unlike common Tet-Off systems with conditional lethal transgenes that are derepressed by withholding tetracycline, a positive activation of the Hsp70Bb promoter is achieved by an elevated temperature and a heat-shock reducing maintenance cost of transgenic lines and facilitating quality control for high-throughput insect rearing. Finally, the Hsp70Bb promoter does not support expression in germ cells even in response to heat-shock stimulation. In fact, the basic promoter sequence of Hsp70Bb, which is incorporated in an upstream activation sequence (UASt) in the Ga14/UAS two-component activation system, is targeted by Piwi-interacting RNAs (piRNAs) in female germ cells leading to degradation of any mRNA harboring endogenous Hsp70Bb gene sequences. The absence of Hsp70Bb-directed expression in germ cells is especially beneficial because a leaky Cas9 expression will not generate inheritable resistance alleles (Hammond et al. PLoS Genet. 13, e1007039 (2017); Oberhofer et al. Proc. Natl. Acad. Sci. U.S.A. 115, E9343-E9352 (2018); KaramiNejadRanjbar et al. Proc. Natl. Acad. Sci. U.S.A. 115, 6189-6194 (2018); and Kandul et al. 2020) at the targeted sequences inside sxl, tra, and βTub genes. The piRNA-mediated degradation of mRNA molecules harboring Hsp70Bb sequences in germ cells safeguards against accumulation and selection of resistance alleles. The described results indicate the Cas9 expression in somatic tissues is weak under the permissive temperature failing to perturb substantially the sex ratio in one-locus pgSIT transgenic flies.

The heat-shock 70 proteins are extremely conserved in insects and play important roles in helping insects survive under stressful conditions. The Drosophila Hsp70Bb promoter is one of the best studied animal promoters. The promoter was widely used for heat-inducible expression of transgenes in many insect species (Morris et al. Nucleic Acids Res. 19, 5895-5900 (1991); Lycett & Crampton. Gene vol. 136 129-136 (1993); Matsubara et al. Proc. Natl. Acad. Sci. U.S.A. 93, 6181-6185 (1996); and Huynh & Zieler. J. Mol. Biol. 288, 13-20 (1999)), and it can be used to drive the heat-inducible expression of Cas9 in many insect species, especially when the lower basic expression is desirable. Native heat-shock Hsp70Bb promoters can also be used to engineer the stronger inducible circuit for Cas9 expression. For example, Hsp70B promoters were characterized and provided a robust heat-inducible expression of transgenes in the yellow fever mosquito Aedes aegypti, the Medeterrenia fruit fly Ceratitis capitata, and the invasive fruit pest Drosophila suzukii. However, it still remains an open question whether the expression of Hsp70B is suppressed in the germline of other insect species.

The one-locus pgSIT approach addresses two major limitations of the previously described pgSIT. First, the original pgSIT relies on the separate inheritance of two required components, Cas9 endonuclease and multiple gRNAs, that are brought together by a genetic cross and become active in the F₁ progeny; consequently, the original pgSIT has the split-pgSIT design. As a result, two insect transgenic lines harboring Cas9 endonuclease and multiple gRNAs genes must be maintained for the split-pgSIT. Second, while the F₁ progeny undergoes autonomous sex-sorting and sterilization during the development and thus can be released as eggs, precise sex-sorting of parental Cas9 and gRNAs virgin flies are required for the genetic cross leading to the production of the F₁ eggs. With a few species-specific exceptions, insect sex-sorting is still time consuming, labor intense, error prone, and species-dependent process. These two limitations constrain the high-throughput application of the split-pgSIT technology for insect population control. The one-locus pgSIT relies on the inducible expression of Cas9 under the Hsp70Bb promoter and pure-breeding of one transgenic insect line. The ability to pure-breed a single one-locus pgSIT line reduces costs associated with insect maintenance and obsoletes laborious sex-sorting. At the same time, the one-locus pgSIT approach retains all the benefits of the pgSIT technology, such as its non-invasiveness, high efficiency, and adaptability to different insect species. Therefore, the inducible one-locus pgSIT approach is the viable strategy for the further improvement of insect population control.

As the original split-pgSIT approach, the one-locus pgSIT genetic cassette in principle can be engineered and applied in any insect species with an annotated genome and established transgenesis protocols. Both one-locus and split-pgSIT approaches use the CRISPR technology to knockout the genes that are conserved across insect taxonomic boundaries, such as genes required for sex-determination and male fertility. The CRISPR technology works in diverse species from bacteria to humans. The tra and dsx genes form the core sex determination pathway, and multiple conserved genes, including βTub, are required for sperm maturation in insects. In fact, the one-locus pgSIT genetic cassettes used in the study can be easily adapted for the invasive fruit pest Drosophila suzukii (Asplen et al. J. Pest Sci. 88, 469-494 (2015)) by changing the gRNA sequences to match the native tra and βTub loci and moving it into a plasmid for piggyBac-mediated integration. The remaining parts, such as Drosophila melanogaster Hsp70Bb and U6.3 promoter as well as the SpCas9 gene and 3′UTRs, are fully active in D. suzukii (Li, F et al., Biochem. Biophys. Res. Commun. 469, 911-916 (2016)). The fact that the one-locus pgSIT approach requires the establishment of a single transgenic line, as an opposite of two lines, facilitates its transgenesis and application for population suppression of insect species. In summary, the described one-locus pgSIT approach is a versatile and powerful technology, and it expedites the development of sustainable and yet confinable measures to control and suppress insect vectors of diseases and agricultural pests.

Assembly of genetic constructs. All genetic constructs generated in this study were built using the Gibson enzymatic assembly. To assemble Hsp70Bb-Cas9 (FIG. 8A), the Rcd1r-Cas9 plasmid was digested with NotI and XhoI to remove the Rcd1r promoter, and the 476-base-long fragment encompassing the Hsp70Bb promoter and cloning overhangs was PCR amplified from the pCaSpeR-hs plasmid (GenBank #U59056.1) using 1137.C1F and 1137.C3R primers and cloned inside the linearized plasmid. The dgRNAs TraB,βTub plasmid was assembled following the strategy used to build dgRNA Sxl,βTub (FIG. 8B). Briefly, the U6.3-gRNA TraB fragment was PCR amplified from the sgRNA TraB plasmid using 2×gRNA-5F and 2×gRNA-6R primers, and cloned into the sgRNA βTub plasmid (addgene #112691). To build the pgSIT Sxl,βTub,Hsp70Bb-Cas9 and pgSIT TraB,βTub,Hsp70Bb-Cas9 constructs (FIG. 8C), the U6.3 3′UTR fragment amplified using 1098A.C1F and 1098A.C2R primers from the pVG185_w2-y1 plasmid (GenBank #MN551090.1) (Kandul et al. 2020; Lopez Del Amo et al., Nat. Commun. 11, 352 (2020)) and the Hsp70Bb-Cas9-T2A-eGFP-10 fragment amplified using 1098A.C3F and 1098A.C6R primers from the Hsp70Bb-Cas9 plasmid, and both were cloned into the dgRNA Sxl,βTub (AddGene #112692) or dgRNA TraB,βTub plasmid, respectively, linearized at XbaI. The gRNA and primer sequences used for assembly of the genetic constructs used in the study are presented in Table 1.

Fly transgenesis. Embryo injections were carried at Rainbow Transgenic Flies, Inc. (www.rainbowgene.com). φC31-mediated integration was used to insert the Hsp70Bb-Cas9 construct at the PBac{y+-attP-3B}KV00033 site on the 3^{rd} chromosome (BDSC #9750); dgRNA TraB,βTub, pgSIT Sxl,βTub,Hsp70Bb-Cas9, and pgSIT TraB,βTub,Hsp70Bb-Cas9 constructs at the P{CaryP}attP1 site on the 2nd chromosome (BDSC #8621); and pgSIT Sxl,βTub,Hsp70Bb-Cas9, and pgSIT TraB,βTub,Hsp70Bb-Cas9 constructs at the P{CaryP}attP2 site on the 3rd chromosome (BDSC #8622). The embryos injected with the pgSIT Sxl,βTub,Hsp70Bb-Cas9 and pgSIT TraB,βTub,Hsp70Bb-Cas9 constructs and any of their progeny starting from the G1 progeny were maintained at +18° C. Recovered transgenic lines were balanced on the 2nd and 3rd chromosomes using single-chromosome balancer lines (w 1118; CyO/sna Sco for II and w 1118; TM3, Sb 1/TM6B, Tb 1 for III).

Fly maintenance and genetics. Flies were examined, scored, and imaged on the Leica M165FC fluorescent stereo microscope equipped with the Leica DMC2900 camera. Inheritance of Hsp70Bb-Cas9 was followed using the Opie2-dsRed genetic marker. The other transgenes were tracked using the mini-white marker. All genetic crosses were done in the w-genetic background. The flies harboring both Hsp70Bb-Cas9 and dgRNAs in the same genetic background were maintained at 18° C. with a 12H/12H light and dark cycle, while the flies harboring either Hsp70Bb-Cas9 or dgRNAs were raised under standard conditions at 26° C. All genetic crosses were done in fly vials using groups of seven-ten flies of each sex. To assess the inducible expression of Hsp70Bb-Cas9, the Cas9/dgRNA knockout phenotypes induced by a heat shock was compared to the same phenotypes without the heat shock. First, three different of dgRNAs (dgRNA sxl,βTub, dgRNA tra,βTub, and gRNA traB,βTub) lines were tested with the same Hsp70Bb-Cas9 line using the classic pgSIT (split-design). The homozygous gdRNAs and Cas9 lines were genetically crossed, and the generated trans-heterozygous embryos were raised at either +20° C. or +26° C. In addition, groups of these embryos underwent various lengths of heat shocks with +37° C. during the 1st or 2nd day after the egg was laid or after the emergence of adult flies, around 10 days at 26° C. (FIG. 9). For heat shocking, glass vials with staged embryos or one-day-old adults were incubated in a water bath at +37° C. Different temperature profiles were tested to assess the induction scale between the leaky and complete expression of Cas9 for each dgRNA construct. Two one-locus inducible pgSIT systems were built with the dgRNA sxl,βTub and gRNA traB,βTub constructs that resulted in the highest induction scale. The transgenic lines harboring one or two copies of pgSIT Sxl,βTub,Hsp70Bb-Cas9 and pgSIT TraB,βTub,Hsp70Bb-Cas9 constructs were constructed, and maintained at +18° C. for a few generations before assessing their induction under +26° C. using a one- or two-hour heat shock at the 1st or 3rd day after egg laying (FIG. 10). To estimate the efficiency of knockout phenotypes induced by either split-pgSIT or one-locus pgSIT systems, the sex of emerging adult flies was scored as female (♀), male (♂), or intersex ( [symbol] ), and tested the fertility of generated flies as previously described in Kandul et al. (Kandul et al, 2019). Note that the induced male sterility was tested in multiple batches of 7-20 males from the same biological sample, and a single fertile male would designate an entire sample as fertile. Each experimental test was repeated at the minimum three times to generate means and standard deviations for statistical comparisons and thus measure consistency and robustness of the results.

Genotyping loci targeted with gRNAs. The molecular changes that caused female lethality and male sterility were examined following the previously described protocol (Kandul et al., 2019). Briefly, the sxl, tra, βTub loci targeted by gRNAs were PCR amplified from individual flies, and sequenced in both directions with Sanger method at GENEWIZ®, and sequence reads were aligned against the corresponding reference sequences in SNAPGENE®. The primer sequences used for PCR of the sxl, tra, βTub loci are presented in Table 1.

Statistical analysis. Statistical analysis was performed in IMP 8.0.2 by SAS Institute Inc. Three to five biological replicates were used to generate statistical means for comparisons. P values were calculated for a two-sample Student's t test with equal variance. To test for significance of male sterilization, Pearson's chi-squared tests for contingency tables were used to calculate P values.

Data Availability. The plasmids constructed in the disclosure were deposited at Addgene.org (#149424-149427, FIG. 8). The Hsp70Bb-Cas9, pgSIT Sxl,βTub,Hsp70Bb-Cas9 and pgSIT TraB,βTub,Hsp70Bb-Cas9 transgenic lines was deposited at Bloomington Drosophila Stock Center.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A gene editing system comprising:
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell.

2. One or more of: a genetically modified insect egg or a progeny thereof, a genetically modified insect or a progeny thereof, or an insect population comprising at least one genetically modified insect or a progeny thereof, comprising the gene editing system of claim 1, optionally wherein the insect is selected from Drosophila melanogaster, Aedes aegypti, Aedes albopictus, Ceratitis capitate, or Drosophila suzukii, and further optionally wherein the insect egg or the insect comprises a polynucleotide of pgSIT^{sxl,βTub, Hsp70Bb-Cas9} or a polynucleotide of pgSIT^{traB,βTub, Hsp70Bb-Cas9}.

3. The insect egg or the insect or the insect population or a progeny of each thereof of claim 2, wherein the insect egg or the insect comprises one or two or more copies of any of:
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell.

4. The insect egg or the insect or the insect population or a progeny of each thereof of claim 2, wherein the insect egg or the insect comprises a contiguous polynucleotide comprising any two, any three, any four, any five, or all of:
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell,
optionally wherein the contiguous polynucleotide further comprises a detectable or selectable marker or a polynucleotide encoding a detectable or selectable marker, optionally wherein the contiguous polynucleotide further comprising a sequence encoding a self-cleaving peptide between the polynucleotide of (a) and the polynucleotide encoding a detectable or selectable marker, and optionally wherein the contiguous polynucleotide further comprises a polyA sequence.

5. (canceled)

6. The insect egg or the insect or the insect population or a progeny of each thereof of claim 2, wherein
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell,
are engineered to one or more of the chromosome(s) or chromosome site(s) of the insect egg or the insect, optionally wherein the insect egg or the insect comprises homozygous (a)-(f), or optionally wherein the insect egg or the insect comprises a heterozygous (a)-(f).

7. The insect egg or the insect or the insect population or a progeny of each thereof of claim 2, wherein expression of the endonuclease is not activated and the insect egg or the insect or the insect population or a progeny of each thereof of is kept under a permissive temperature of the regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter, optionally wherein the permissive temperature is about 18° C., and optionally wherein the progeny is the first, second, third, fourth, fifth, sixth, seventh, eighth, ninth, tenth, or more generations of the insect egg or insect or insect population.

8. The insect egg or the insect or the insect population or a progeny of each thereof of claim 2, wherein expression of the endonuclease is activated by keeping the insect egg, or the insect or the insect population or a progeny of each thereof under a restrictive temperature of the regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter, optionally wherein the restrictive temperature is about 26° C. or higher, optionally wherein the progeny comprises at least about 80%, or at least about 85%, or at least about 90%, or at least about 95%, or at least about 96%, or at least about 97%, or at least about 98%, or at least about 99%, or up to 100% sterile male.

9. The insect egg or the insect or the insect population or a progeny of each thereof of claim 2, wherein the expression of the endonuclease is activated by one or more of heat-shock(s), optionally wherein the heat-shock is at about 37° C., and further optionally wherein the heat-shock is about 1 hour long, or about 2 hours long, or about 3 hours long, or about 4 hours long.

10. (canceled)

11. The insect egg or the insect or the insect population or a progeny of each thereof of claim 2, having at least about 50%, or at least about 75%, or at least about 80%, or at least about 85%, or at least about 90%, or at least about 95%, or at least about 96%, or at least about 97%, or at least about 98%, or at least about 99%, or up to 100% of the fitness or the mating competitiveness of a wild type.

12. (canceled)

13. An isolated or engineered polynucleotide comprising any two, any three, any four, any five, or all of the following:
(a) a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini;
(b) a regulatory sequence directing the endonuclease expression in a cell, optionally wherein the cell is an insect germline cell, optionally wherein the regulatory sequence is temperature-sensitive, and further optionally wherein the regulatory sequence comprises a heat-shock protein 70B (Hsp70Bb) promoter;
(c) a guide polynucleotide targeting a female-essential genomic sequence that is required for female-specific viability, or a complementary sequence of the guide polynucleotide, or a polynucleotide expressing the guide polynucleotide, optionally wherein the guide polynucleotide targeting a sex-specifically alternatively spliced sex-determination gene optionally selected from: sex lethal (Sxl), transformer (tra), or doublesex (dsxF);
(d) an optional regulatory sequence directing expression of the guide polynucleotide of (c) in a cell, optionally wherein the regulatory sequences is a RNA pol III promoter, optionally selected from the group consisting of H1, U6, and U6.3, and optionally wherein the cell is an insect germline cell;
(e) a guide polynucleotide targeting a male sterility genomic sequence that is required for male-specific fertility, or a complementary sequence of the guide polynucleotide, or a polynucleotide expressing the guide polynucleotide, optionally wherein the guide polynucleotide targets a gene active during spermatogenesis optionally selected from βTubulin 85D (βTub), fuzzy onions (fzo), protamine A (ProtA), or spermatocyte arrest; and
(f) an optional regulatory sequence directing expression of the guide polynucleotide of (e) in a cell, optionally wherein the regulatory sequences is a RNA pol III promoter, optionally selected from the group consisting of H1, U6, and U6.3, and optionally wherein the cell is an insect germline cell,
optionally wherein the isolated or engineered polynucleotide comprises more than one copy of any of (a)-(f).

14. (canceled)

15. The isolated or engineered polynucleotide of claim 13, further comprising one or more of the following: a polynucleotide encoding a detectable or selectable marker, a sequence encoding a self-cleaving peptide between a polynucleotide encoding an endonuclease, optionally wherein the endonuclease is Cas9, optionally wherein the polynucleotide further encodes a nuclear localization signal at the amino terminus of the endonuclease or the carboxyl terminus of the endonuclease or both termini and the polynucleotide encoding a detectable or selectable marker, and a polyA sequence.

16. The isolated or engineered polynucleotide of claim 13, comprising a polynucleotide of pgSIT^{sxl,βTub, Hsp70Bb-Cas9} or a polynucleotide of pgSIT^{traB,βTub, Hsp70Bb-Cas9} or both.

17. A vector comprising one or more of the polynucleotide(s) of claim 13.

18. An isolated or engineered host cell comprising the polynucleotide of claim 13, optionally producing copies of the polynucleotide of claim 13.

19. (canceled)

20. The host cell of claim 18, wherein the host cell is an insect cell, and further optionally wherein the host cell is selected from an egg, a sperm, a zygote, or a germline cell.

21. The host cell of claim 18, wherein the polynucleotide is engineered to one or more of the chromosome(s) or chromosome sites of the host cell, optionally wherein the host cell comprises homozygous polynucleotides, or optionally wherein the host cell comprises a heterozygous polynucleotide.

22. A method of reducing a wild-type insect population comprising introducing an insect egg or an insect or an insect population or a progeny of each thereof of claim 2, to the wild-type insect population.

23. A method of producing (1) a genetically modified insect egg, (2) a genetically modified insect, (3) a population comprising the genetically modified insect egg or the genetically modified insect, (4) a population comprising substantially male insect egg or male insect or both, (5) or a progeny of each thereof, comprising introducing the gene editing system of claim 1 into an insect egg, or an insect, or a population of each thereof, or a progeny of each thereof, optionally keeping the insect egg, the insect, the population or the progeny under a restrictive temperature, and further optionally heating shocking the insect egg, the insect, the population or the progeny.

24. (canceled)

25. (canceled)

26. A composition comprising a carrier and the system of claim 1.

27. A kit comprising an instruction of use in the method of claim 22.

28. (canceled)
